(19) United States
(12) Patent Application Publication
(10) Pub. No.: US 2023/0026808 A1
(43) Pub. Date: Jan. 26, 2023

(54) COMPOUNDS, COMPOSITIONS, AND METHODS FOR TREATING ISCHEMIA-REPERFUSION INJURY AND/OR LUNG INJURY

(71) Applicant: YALE UNIVERSITY, New Haven, CT (US)

(72) Inventors: Dianqing WU, New Haven, CT (US); Qianying YUAN, Guilford, CT (US); Wenwen TANG, North Haven, CT (US)

(21) Appl. No.: 17/756,212

(22) PCT Filed: Nov. 17, 2020
(86) PCT No.: PCT/US20/60906

§ 371 Date: May 19, 2022

Related U.S. Application Data
(60) Provisional application No. 62/938,083, filed on Nov. 20, 2019.

(51) Int. Cl. A61K 31/506; A61P 11/00; A61P 9/10

(52) U.S. Cl. CPC A61K 31/506; A61P 11/00; A61P 9/10

(57) ABSTRACT

The disclosure includes methods of preventing, ameliorating, and/or treating ischemia-reperfusion injury (IRI), including but not limited to post brain stroke, using a MAP3K2/MAP3K3 inhibitor. In another aspect, the present disclosure relates to methods of preventing, ameliorating, and/or treating a lung injury related to a coronavirus infection, acute lung injury (ALI) and/or acute respiratory distress syndrome (ARDS) using a MAP3K2/MAP3K3 inhibitor. The disclosure further comprises compositions, and kits comprising compositions useful within the disclosure.

### CROSS-REFERENCE TO RELATED APPLICATIONS

This application claims priority under 35 U.S.C. § 119(e) to U.S. Provisional Patent Application No. 62/938,083, filed Nov. 20, 2019, which is incorporated herein by reference in its entirety.

### STATEMENT REGARDING FEDERALLY SPONSORED RESEARCH OR DEVELOPMENT

This invention was made with government support under HL135805 and awarded by National Institutes of Health. The government has certain rights in the invention.

### BACKGROUND OF THE DISCLOSURE

Ischemia-reperfusion injury (IRI) occurs when blood supply is restored after a period of ischemia. In the case of brain stroke, reperfusion can be achieved either by thrombolysis triggered by thrombolytic reagents, such as tissue plasminogen activator (tPA), or through mechanical removal of thrombi. Spontaneous reperfusion also occurs after ischemic stroke. Reperfusion restores oxygen supply to the affected tissue, and unfortunately this has deleterious effects compared with permanent ischemia.

Reperfusion injury following ischemic stroke is a complex pathophysiological process involving numerous mechanisms such as, but not limited to, release of excitatory amino acids, ion disequilibrium, oxidative stress, inflammation, apoptosis induction, and/or necrosis. With the recent advancements in endovascular therapy (including thrombectomy and thrombus disruption), reperfusion injury has become an increasingly critical challenge in stroke treatment. It is thus of extreme importance to understand the mechanism of ischemia-reperfusion injury in the brain and how this process can be therapeutically managed without unnecessary cell and tissue damage.

A novel coronavirus has emerged as the infectious agent that afflicted a large number of people in Wuhan, China in December 2019. The virus has been designated as severe acute respiratory syndrome coronavirus 2 (SARS-CoV-2), which is the causative agent for coronavirus disease 2019 (COVID-19). This disease quickly spread worldwide to pandemic levels.

The upper respiratory tract and lungs are the main points of viral entry and replication for SARS-CoV-2, and respiratory illness is the primary manifestation of the associated disorder, COVID-19, as well as a major cause of death, although other organ systems are also affected. As COVID-19 progresses, it commonly presents with severe lung edema, a manifestation of acute lung injury (ALI), and can further progress to severe hypoxemia and acute respiratory distress syndrome (ARDS). Notably, the occurrence and severity of ALI has shown an association with the prognosis of SARS-CoV-2 infected individuals, and ALI/ARDS is reportedly central to the pathophysiology of COVID-19 progression to multi-organ dysfunction and death. Some distinct manifestations have been reported for COVID-19 related ARDS, for example, relatively normal lung compliance in the presence of severe hypoxemia. However, the differences may be viewed as reflective of the broad heterogeneity of the syndrome itself, and it has been suggested that emerging evidence indicates broad similarity in respiratory system mechanics for both historic and coronavirus infection associated ARDS. Thus, a treatment with potential clinical benefits in ALI/ARDS would be anticipated to reduce the severity of coronavirus infection (e.g. COVID-19) and improve overall survival in affected patients, both in patients wherein the coronavirus infection has progressed to ALI/ARDS and in patients with a coronavirus infection that affects the lungs and/or respiratory tract but which has not progressed to ALI/ARDS.

Acute lung injury (ALI) and its more severe form acute respiratory distress syndrome (ARDS), which are caused by direct or indirect insults to the lung, which may be associated with a coronavirus infection or other causes such as, but not limited to, lipopolysaccharide (LPS)-induced ALI/ARDS, aspiration-induced ALI/ARDS, ALI/ARDS caused by ischemia reperfusion, and/or bacterial/viral ALI/ARDS. Regardless of the cause of ALI/ARDS, this lung injury represents a serious health problem with a high mortality rate. The incidence of ALI/ARDS is reported to be around 200,000 per year in the US with a mortality rate of around 40%. Currently there are no pharmacological interventions for the diseases. Care of these conditions is largely dependent on supportive measures. Pharmacological therapies that have been tested in patients with ALI/ARDS failed to show efficacy. There is thus a clear unmet medical need for therapeutic intervention of this disease.

MAP3K2 and MAP3K3 are two highly conserved members of the MEK kinase (MEKK) subgroup of the MAP3K superfamily. They contain a kinase domain in the C terminus and a PB1 domain near the N terminus. The kinase domains of MAP3K2 and MAP3K3 share 94% sequence identity, and these two kinases are expected to share substrates. Transient expression of the kinases in vitro leads to their auto-activation and activation of ERK1 and ERK2, p38, INK, and ERK5. In mice, these kinases are involved in cardiovascular development, lymphocyte differentiation, and NF-kappaB regulation. However, their roles in other physiological events have not been investigated.

There is a need in the art to identify novel therapeutic treatments that can be used to treat, ameliorate, and/or prevent ischemia-reperfusion injury, lung injury related to a coronavirus infection, acute lung injury, and/or acute respiratory distress syndrome in afflicted subjects. In certain embodiments, the ischemia-reperfusion afflicted subjects have suffered ischemic stroke. In certain embodiments, the lung injury related to a coronavirus infection has progressed to acute lung injury and/or acute respiratory distress syndrome. In other embodiments, the lung injury related to a coronavirus infection has not progressed to acute lung injury and/or acute respiratory distress syndrome. The present disclosure addresses and meets this need.

### BRIEF SUMMARY OF THE DISCLOSURE

The disclosure provides a method of treating, ameliorating, and/or preventing post-stroke brain ischemia-reperfusion injury (IRI) in a subject in need thereof. In certain embodiments, the method comprises administering to the subject a therapeutically effective amount of pazopanib, and/or a salt and/or solvate thereof.

The disclosure further provides a method of treating, ameliorating, and/or preventing ischemia-reperfusion injury (IRI) not caused by post-stroke brain ischemia, lung injury related to a coronavirus infection, acute lung injury (ALI), and/or acute respiratory distress syndrome (ARDS) in a subject in need thereof. In certain embodiments, the method comprises administering to the subject a therapeutically effective amount of pazopanib, and/or a salt and/or solvate thereof.

The disclosure further provides a method of evaluating efficacy of a drug in treating ischemia-reperfusion injury (IRI), lung injury related to a coronavirus infection, acute lung injury (ALI), and/or acute respiratory distress syndrome (ARDS). In certain embodiments, the method comprises contacting a neutrophil with the drug and measuring neutrophil ROS production levels after the contacting, wherein, if the neutrophil ROS production levels increase after the contacting, the drug is efficacious in treating IRI, lung injury related to the coronavirus infection, ALI, and/or ARDS.

The disclosure further provides a method of evaluating efficacy of a drug in treating a subject suffering from ischemia-reperfusion injury (IRI), lung injury related to a coronavirus infection, acute lung injury (ALI), and/or acute respiratory distress syndrome (ARDS). In certain embodiments, the method comprises (i) measuring neutrophil ROS production levels in the subject after being administered the drug, wherein, if the neutrophil ROS production levels in the subject after being administered the drug are higher than the neutrophil ROS production levels in the subject before being administered the drug, the drug is efficacious in treating IRI, lung injury related to the coronavirus infection, ALI, or ARDS in the subject; and/or (ii) measuring H₂O₂ levels in the lungs of the subject after being administered the drug, wherein, if the H₂O₂ levels in the lungs of the subject after being administered the drug are higher than the H₂O₂ levels in the lungs of the subject before being administered the drug, the drug is efficacious in treating lung injury related to the coronavirus infection, ALI or ARDS in the subject.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 illustrates that pazopanib reduces brain IRI in an intraluminal middle cerebral artery (MCA) occlusion brain stroke mouse model. Thirteen female C57bl mice (9 weeks old) were subjected to the occlusion for 60 min before reperfusion of blood was allowed. Seven of them were administered with 60 μg of pazopanib via retro-orbital intravenous injection 30 min after reperfusion. The animals were scored for neurological damage (the bar chart at lower right) 24 hour late before they were euthanized. The brain infarction was then evaluated by staining the brain slices with TCC. TCC stained images are shown and infract sizes were quantified and are shown in the bar chart at upper right.

FIG. 2 illustrates that pazopanib fails to reduce brain IRI in an intraluminal middle cerebral artery (MCA) occlusion brain stroke mouse model if the pazopanib was administrated at the time of reperfusion. Six female C57bl mice (9 weeks old) were subjected to the occlusion for 60 min before reperfusion of blood was allowed. Three of them were administered with 60 μg of pazopanib via retro-orbital intravenous injection immediately after reperfusion. Neurological damage score (bar chart at lower right), TCC-stained brain slice images and brain infarct size quantification are shown at upper right.

FIGS. 3A-3F depict that MAP3K2/3-null neutrophils show normal functions except ROS (reactive oxygen species) production. FIG. 3A: Loss of MAP3K2 and 3 proteins in the DKO neutrophils. Bone marrow neutrophils were analyzed by Western using MAP3K2 and 3-specific antibodies, respectively. FIG. 3B: ROS release from WT and MAP3K2/3-deficient bone marrow neutrophils in presence of 1 μM fMLP. FIG. 3C: ROS amounts from isolated neutrophils calculated from the areas under the traces for 5 min after stimulation as shown in B are shown (data are presented as mean±sem, One-Way Anova; n=5). K2 and K3 stands for Map3k2^{-/-} and Map3k3^{-/-}, respectively. FIG. 3D: MAP3K2/3-deficiency increases ROS production from mouse neutrophils stimulated by two doses of fMLP as well as by MIP2 (in μM). Data are presented as mean±sem (***, p<0.001; Student's t-test; n=3). FIG. 3E: ROS production from bone marrow neutrophils stimulated by 1 μM fMLP was measured using cytochrome C assay. FIG. 3F: Expression of WT MAP3K3, but not its kinase dead mutant, suppresses ROS production in DKO neutrophils. Neutrophils were transiently transfected with plasmids for GFP, MAK3K3-GFP, or MAP3K3 kinase dead (KD) fused with GFP. GFP-positive cells were sorted the next day and used for ROS release assay. Data are presented as mean±sem (One-way Anova test, n=3).

FIGS. 4A-4P depict the effects of MAP3K2 and 3-deficiency on neutrophil functions. FIGS. 4A-4D: Neutrophils were subjected to Dunn chamber chemotaxis under stimulation of fMLP. Representative cell migration traces are shown in (FIGS. 4A & 4B). The translocation and directionality parameters for how fast the cells move and how well they follow the chemoattractant gradient are shown in (FIG. 4C) and (FIG. 4D). Data are presented as mean±sem (Student t-Test, n>50). DKO, Map3k2^{-/-}, Map3k3^{-/-}. FIG. 4E: Adhesion of neutrophils to endothelial cells was examined in a shear flow chamber. FIGS. 4F-4G: Cell surface expression of LFA-1 and MAC-1 integrins on neutrophils stimulated with fMLP. FIG. 4H: Binding of neutrophils to ICAM-1, which reflects the avidity of integrins on neutrophils upon activation by fMLP. FIG. 4I: Infiltration of neutrophils into inflamed peritonea. FIGS. 4J-4K: Release of MMP and MPO from neutrophils granules upon stimulation. FIG. 4L: ROS production from neutrophils stimulated by 1 μM fMLP was measured using luminol in the buffer (0.25% BSA in HBSS with Ca²⁺ and Mg²⁺, 10 mM Isoluminol, 100 u/ml HRP). FIGS. 4M and 4N: Neutrophils from peritoneal and bone marrows were isolated using EasySep™ Mouse Neutrophil Enrichment Kit (Stemcell Tech) and stimulated by 1 μM fMLP before ROS was measured using isoluminol. FIG. 4O: ROS production from neutrophils stimulated by 200 nM PMA was measured using isoluminol. Data in FIGS. 4E-4O are presented as mean±sem (Student's t-test). FIG. 4P: The expression of MAP3K3 and its mutants were detected by Western analysis in support of FIG. 3F.

FIGS. 5A-5G depict that loss of MAP3K2 in hematopoietic cells and MAP3K3 in myeloid cells ameliorates acute lung injury. FIGS. 5A and 5D: Reduced pulmonary permeability in DKO mice. DKO and control WT mice were subjected to HCl or LPS-induced ALI, followed with pulmonary permeability measurement. Data are presented as mean±sem (Student t-Test, n=8). FIGS. 5B and 5E: Representative histology of injured lungs. Br, bronchus; V, blood vessel; yellow circles denote areas of perivascular interstitial edema. Quantification for perivascular interstitial edema and ALI index is shown in FIG. 6A. FIGS. 5C and 5F: DKO mice show extended survival (the Mantel-Cox Log-Rank test; n=5; p=0.004). FIG. 5G: Neutrophils from HCl-injured lungs and BALs of DKO mice produce greater amounts of ROS than those of WT mice. Data (mean fluorescence intensity) are presented as mean±sem (Student's t-test, n=4).

FIGS. 6A-6F depict the effects of MAP3K2 and 3-deficiency on ALI. FIG. 6A: Quantification of perivascular interstitial edema and lung injury index for FIGS. 5A-5G. FIG. 6B: Effect of MAP3K2 (K2) or MAP3K3 (K3) deficiency on pulmonary permeability in the HCl-induced ALI model. FIG. 6C: Myeloid cell presence in BALs of HCl-injured lungs of DKO and WT mice. Absolute cell numbers are shown. FIG. 6D: Myeloid cell infiltration in HCl-injured lungs of DKO and WT mice. Lungs were perfused with PBS and digested with collagenase before flow analyses. Data shown were pre-gated with CD45. Absolute cell numbers are shown. FIG. 6E: The numbers of circulating blood cells in DKO and WT mice post HCl-induced ALI. FIG. 6F: Cytokine levels in BAL of HCl-injured lungs of DKO and WT mice. Data in FIGS. 6A-6F are presented as mean±sem (Student's t-test).

FIGS. 7A-7I: depict that MAP3K3 phosphorylates p47^{phox} at S208 to inhibit NADPH oxidase activity. FIG. 7A: MAP3K3 phosphorylates p^{47phox}. In vitro kinase assay was performed using purified recombinant MAPK3K3 and immunoprecipitated NADPH oxidase subunits. The NADPH oxidase subunits were transiently expressed in HEK293 cells with an HA-tag, and an anti-HA antibody was used for immunoprecipitation. FIG. 7B: MAP3K3 phosphorylates S208 of p47^{phox}. In vitro kinase assay was performed using recombinant MAP3K3 and GST-p47SH3 (WT) or GST-p47SH3 containing a S208E mutation (SE). GST-p47SH3 is a glutathione S-transferase-fused p47^{phox} fragment (residues 151-286) that contains the two SH3 domains. FIG. 7C: Phosphomimetic mutation of Ser-208 of p47^{phox} leads to reduced activity in the reconstituted ROS production assay. COS-7 cells were cotransfected with plasmids for p22^{phox}, p67^{phox}, and p97^{phox} together with WT p47^{phox} or its S208A (SA) or S208E (SE) mutant. The PMA-induced ROS production are shown. Data are presented as mean±sem (Two sided one-way Anova, n=4). FIG. 7D: WT p47^{phox}, but not its S208A mutant, is inhibited by MAP3K3. COS-7 cells were cotransfected with plasmids for p22^{phox}, p67^{phox}, and p97^{phox} together with WT p47^{phox} (left panel) or its S208A mutant (right panel) in the presence or absence of MAP3K3. The PMA-induced ROS production are shown. Data are presented as mean±sem (Student t-Test). FIG. 7E: Phosphomimetic mutation of Ser-208 of p47^{phox} impairs the interaction with p22^{phox}. GST pull-down assay was performed with GST-p47SH3 S208A or S208E mutant and MBP-fused C-terminus (residues 96-164) of p22^{phox} (p22C). Western analysis was used for detection of the proteins. FIG. 7F: Phosphorylation of Ser-208 of p47^{phox} is stimulated by fMLP. Neutrophils were stimulated with fMLP (1 μM) for varying durations, followed by Western analysis. FIG. 7G: FMLP-stimulated p47^{phox} phosphorylation depends on MAP3K2/3. FIG. 7H: Neutrophils from p47^{phox}-KI mice release more ROS than WT. Data are presented as mean±sem (Student t-Test, n>5). FIG. 7I: The p47^{phox}-KI mice have reduced pulmonary permeability post HCl-induced ALI. Data are presented as mean±sem (Student t-Test, n>4).

FIGS. 8A-8I depict that MAP3K2/3 regulates NADPH oxidase complex 2 by phosphorylation of p47^{phox}. FIGS. 8A-8B: COS-7 cells were transfected with plasmids for NADPH oxidase subunits as indicated in the figure and treated with and without PMA. ROS production and protein expression were determined. FIG. 8C: WT MAP3K3, but not its kinase dead mutant, can inhibit ROS production in the reconstituted COS-7 system. Data are presented as mean±sem (One-way Anova, n=4 for LacZ and 3 for others). FIG. 8D: A schematic model depicts how MAP3K2/3 suppresses ROS production. MAP2K2/3 phosphorylates p47^{phox} at Ser-208. The phosphorylation interferes with the interaction between p47^{phox} with p22^{phox} and thus inhibits the NADPH oxidase activity and ROS production. FIG. 8E: Validation of the anti-phospho-S208 p47^{phox} antibody. HEK293 cells were cotransfected with WT together with WT or S208A p47^{phox}. Western analysis was performed the next day. FIGS. 8F-8G: Quantification of Western blots in FIGS. 7F and 7G. Data are presented by normalized values of p-p47 over total p47. n=3. FIGS. 8H-8I: Validation of p47^{phox} S208 A knock in by DNA sequencing (FIG. 8H; top, SEQ ID NOs:2-3, and bottom, SEQ ID NOs:4-5) and Western analysis (FIG. 8I). Neutrophils from WT and p47^{phox}-KI (KI) mice were stimulated with fMLP (1 μM) for times indicated and analyzed by Western blotting in FIG. 8I.

FIGS. 9A-9L depict the alteration of pulmonary microenvironments by p47^{phox}-KI. FIG. 9A: t-SNE plots of single cell RNA sequencing of lung CD45-negative cells. FIG. 9B: pathway enrichment analysis of endothelial cells. Only those that are related to Akt signaling are shown. FIG. 9C: Lung sections from WT and MAP3K2/3 DKO mice were stained for phospho-S473 AKT (pAKT) and CD31. Samples were collected 6 hour after ALI induction by HCl. FIG. 9D: Quantification of endothelial cells p-AKT staining demarked by CD31 staining for FIG. 10A. Each datum point is an average of more than 8 vessel sections from one mouse. FIG. 9E: Increases in phosphorylation of AKT at S308 in the protein extracts from HCl-injured lungs of DKO mice compared to those from WT mice. Quantification is shown as mean±sem (Student's t-test). FIG. 9F: Low concentrations of H₂O₂ enhances TEER and stimulates AKT in primary mouse lung endothelial cells. FIG. 9G: Quantification for FIG. 10B. FIG. 9H: Reduced cytochrome C abrogates increased AKT phosphorylation in endothelial cells by co-cultured MAP3K2/3-deficient neutrophils (DKO). FIG. 9I: Co-culture of fMLP-stimulated p47^{phox}-KI causes greater AKT phosphorylation in lung endothelial cells compared to that of fMLP-stimulated WT neutrophils. FIGS. 9J-9L: Intravenous administration of pegylated catalase (Cat; 2000 U/mouse) via tail veins immediately before HCl-induced ALI increases permeability, interstitial edema, and mortality in WT mice. Heated-inactivated (iCat) was used as a control in addition to mock. Data are presented as mean±sem. Data in FIGS. 9D-9G, 9I, 9J, and 9L are presented as mean±sem (Student's t-test).

FIGS. 10A-10I depict alteration of pulmonary microenvironments by p47^{phox}-KI. FIGS. 10A and 10F-10I: Lung sections from WT and p47phox-KI mice were stained for phospho-S473 AKT (pAKT), CD31, smooth muscle actin (SMA), ABCA3, activated caspase 3 (CASP3) and/or Ki67 as indicated in the panels. Samples were collected 6 hours after ALI induction by HCl except for (FIG. 10I), which was collected 24 hours after injury. Representative confocal images are shown. Quantifications are shown in FIGS. 9D, 11B, 12A-12C. FIG. 10B: Co-culture of fMLP-stimulated MAP3K2/3-deficient neutrophils (DKO) causes greater AKT phosphorylation compared to that of fMLP-stimulated WT neutrophils, and this difference in AKT phosphorylation is abrogated by the presence of catalase (Cat), but not superoxide dismutase (SOD). Quantification is shown in FIG. 9G. FIG. 10C: TEER measurement of mouse lung endothelial cells co-cultured with fMLP-stimulated WT or DKO neutrophils in the presence or absence of SOD. FIG. 10D: Intravenous administration of pegylated catalase (2000 U/mouse) via tail veins right before HCl instillation increases permeability and abrogates the effect of MAP3K2/3 deficiency on HCl-induced permeability change. Data are presented as mean±sem (two-way Anova; ns, not significant). FIG. 10E: Violin plots for comparison of gene expression of p47^{phox}-KI (KI) and WT samples using single cell RNA sequencing. EC1 and EC2 are the two endothelial cell subgroups.

FIGS. 11A-11E depict the alteration of pulmonary endothelial microenvironments by p47^{phox}-KI. FIG. 11A: t-SNE plots of single cell RNA sequencing of lung CD45-negative cells. FIG. 11B: Quantification of p-AKT staining marked by SMA staining for FIG. 10F. Each datum point is an average of more than 8 vessel sections from one mouse. FIGS. 11C, 11D, 11E: Violin plots for comparison of gene expression from single cell RNA sequencing of lung CD45-negative cells of p47^{phox}-KI and WT lungs. Data in FIG. 11B are presented as mean±sem (Student's t-test).

FIGS. 12A-12G depict the alteration of pulmonary epithelial microenvironments by p47^{phox}-KI. FIGS. 12A-12C: Quantification of p-AKT, Ki67 or CASP3 staining in ABCA3 positive cells for FIGS. 10G, 10H, and 10I. Each datum point is an average of more than 30 ABCA3-positive cells from one mouse. FIG. 12D: t-SNE plots of single cell RNA sequencing of lung CD45-negative cells. FIGS. 12E-12F: Violin plots for comparison of gene expression from single cell RNA sequencing of lung CD45-negative cells of p47^{phox}-KI and WT lungs. FIG. 12G: Confocal images of ALI lung sections stained with antibodies for PDPN and activated caspase 3 (CASP3). Data in FIGS. 12A-12C and 12G are presented as mean±sem (Student's t-test).

FIGS. 13A-13E depict the effects of Pazopanib on phosphorylation of p47^{phox} by MAP3K2/3 and neutrophils. FIGS. 13A-13B: Effects of varying doses of pazopanib on p47^{phox} phosphorylation by MAP3K2 or 3 in in vitro kinase assays were determined using the anti-phospho-p47^{phox} at S208. Data are presented as mean±sem (n=3 independent experiments; One-way Anova). FIG. 13C: Pazopanib inhibits phosphorylation of Ser-208 of p47^{phox} in neutrophils stimulated by fMILP (1 μM). FIGS. 13D-13E: Pazopanib increases ROS release from fMLP (1 μM)-stimulated neutrophils depending on MAP3K2/3. Data are presented as mean±sem (two-way Anova test, n=4).

FIGS. 14A-14B depict the effects of pazopanib on phosphorylation and human neutrophils. FIG. 14A: Effect of pazopanib on MEK5 phosphorylation by MAP3K2 or 3 in an in vitro kinase assay. Data are presented as mean±sem (One-way Anova). FIG. 14B: Effects of pazopanib on ERK and p38 phosphorylation in mouse neutrophils.

FIGS. 15A-15H depicts that pazopanib ameliorates ALI. FIGS. 15A and 15B: Schematic representation of the therapeutic treatment modality. Mice (C57B1 female, 8 weeks) were treated with 1.5 mg/Kg pazopanib intra-nasally. FIGS. 15C-15F: Pulmonary permeability and histology were examined after injury (data are presented as mean±sem; Student t-Test, n=10). Quantification of perivascular interstitial edema was done as the ratios of interstitial edema areas to vessel areas. Quantification of lung injury is also shown. More than 8 sections from the same lobes of the lungs were quantified for each mouse. Data are presented as mean±sem (Student's t-test; n=5). FIGS. 15G-15H: Therapeutic treatment of pazopanib reduces mortality in ALI models (Mantel-Cox Log-Rank test; n=8).

FIGS. 16A-16J depict that pazopanib ameliorates ALI. FIG. 16A: Neutrophils from BALs and lungs of mice subjected to HCl lung insult that were treated with or without pazopanib were measured for ROS using DCFDA. Data are presented as mean±sem (Student's t-test, n=4). FIGS. 16B-16D: Pazopanib shows no significant effects on neutrophil infiltration in BAL and lungs or BAL cytokine contents. Data are presented as mean±sem. No significance between mock and pazopanib treated (Student's t-test; n=5). FIGS. 16E-16F: Schematic representation of the prophylactic modality. Mice (C57B1 female, 8 weeks) were treated with 60 mg/Kg/day pazopanib via gavage for three days in the LPS model, whereas the mice were treated once with 1.5 mg/Kg pazopanib intra-nasally in the HCl model. FIGS. 16G-16H: Pulmonary permeability was examined after injury. Data are presented as mean sem (Student's t-test; n=5). FIGS. 16I-16J: Mortality was analyzed using Mantel-Cox Log-Rank test.

FIGS. 17A-17E depicts that pazopanib acts through the MAP3K2/3-p47^{phox} pathway. FIGS. 17A, 17B, and 17D: Mice were subjected to treatment as described in FIG. 15A, followed with pulmonary permeability measurements. FIG. 17C: p47^{phox} S208A knock-in increases ROS production and abrogates pazopanib's effect on neutrophils. Neutrophils from WT or p47^{phox}-KI mice were stimulated with fMLP (1 μM) in the presence of absence of 20 nM of pazopanib. FIG. 17E: Intravenous administration of pegylated catalase (2000 U/mouse) via tail veins right before HCl instillation increases permeability and abrogates the effect of pazopanib in HCl-induced permeability change. Data in FIGS. 17A-17E are presented as mean±sem (two-way Anova; ns, not significant).

FIGS. 18A-18C depict the mechanism of action of Pazopanib. FIG. 18A: Pazopanib failed to increase survival in mice lacking p47^{phox}. FIG. 18B: Pazopanib increases phosphorylation of AKT at S473 in ALI lung extracts. Data are presented as mean±sem (Student's t-test). FIG. 18C: AKT inhibitor (MK-2206) abrogates protective effect of pazopanib in HCl-injured lungs (data are presented as mean±sem).

FIGS. 19A-19D depict that pazopanib ameliorates edema in human injured lungs. FIG. 19A: Effect of pazopanib on ROS production from human neutrophils in the presence of 100 nM of fMLP. Data are presented as mean±sem (Student's t-test, n=12). FIG. 19B: Patient information of 5 pairs LT (lung transplantation) recipients. FIG. 19C: Effect of pazopanib on pulmonary edema. *p<0.05 (Linear mixed model repeated measures analysis). FIG. 19D: Representative Chest X-ray images. Chest X-ray examinations were performed on post-operative Day 1 and Day 2. Patient #1a received the left lung, marked in red outline, and did not receive the drug, whereas Patient #1b received the right lung, marked in green outline and received pazopanib, from the same donor. Patient #1b exhibited less lung opacification than Patient #1a on Day 1, with significant improvement by Day 2. Of note, Patient #1b underwent the operation later and had a longer ischemic time than Patient #1a.

FIG. 20 depicts non-limiting percentage permeability for pazopanib IV in the HCl-induced ALI model.

FIG. 21 depicts non-limiting percentage permeability for pazopanib IV in the MHV-1 mouse model (Study 1).

FIG. 22 depicts non-limiting percentage permeability for pazopanib IV in the MHV-1 mouse model (Study 2).

FIG. 23 depicts a non-limiting design diagram for the 2-part Phase 2 Study, wherein Pts refers to participants and QXT-101 refers to pazopanib IV.

### DETAILED DESCRIPTION OF THE DISCLOSURE

The present disclosure relates in part to the unexpected discovery that MAP3K2 and/or MAP3K3 inhibition can be used to treat, ameliorate, and/or prevent ischemia-reperfusion injury (IRI), acute lung injury (ALI), and/or acute respiratory distress syndrome (ARDS).

There is an abundant accumulation of neutrophils during stroke, and reperfusion post-thrombolysis further activates the neutrophils. The migration of neutrophils into the brain parenchyma and release of their abundant proteases are generally considered the main cause of neuronal cell death and contribute to disruption of the blood brain barrier (BBB), cerebral edema, and brain injury. In addition, one of the hallmarks of ALI is abundant presence of neutrophils in the lungs where they play important roles in innate immunity against microbial infections, contribute to inflammation-related tissue damages, and have been clearly linked to pulmonary edema formation. Neutrophils play a predominant role in the expansion of inflammatory tissue damage and disruption of barrier function in ALI/ARDS, and these leukocytes appear to amplify lung injury in COVID-19 as well, which is supported by findings showing neutrophilia as a risk factor in the development of ARDS and progression from ARDS to death in patients with COVID-19. Additionally, increased neutrophil levels have shown an association with disease severity in this population.

Neutrophils produce reactive oxygen species (ROS) primarily through the phagocyte NADPH oxidase, which is a member of the NOX family. It consists of four cytosolic components (p47^{phox}, 67^{phox}, p40^{phox}, and Rac) and two membrane subunits (gp91^{phox}/NOX2 and p22^{phox}). Upon cell activation, the cytosolic components are recruited to the membrane components to form the active holoenzyme to produce ROS. One of the key activation events is the phosphorylation of the cytosolic p47^{phox} subunit by protein kinases including PKC. The phosphorylation disrupts auto-inhibitory intramolecular interaction involving the internal SH3 domains, leading to its interaction with p22^{phox}, required for the activation of the NADPH oxidase. MAP3K2 and MAP3K3 are negative regulators of neutrophil NADPH oxidase by phosphorylating p47^{phox} at Serine 208. This phosphorylation, in contrast to previously known phosphorylation sites in p47^{phox}, prevents p47^{phox} interaction with p22^{phox} and leads to inhibition of the NADPH oxidase activity and inhibition of ROS production. Either the genetic loss of MAP3K2/3 or their pharmacological inhibition results in increased ROS production in neutrophils. The ROS released from neutrophils is converted into H₂O₂, which acts on endothelial cells to enhance its barrier function, curbing inflammatory responses and providing beneficial therapeutic effects.

As demonstrated herein, pazopanib, which inhibits MAP3K2/3 activity, increases ROS production in neutrophils and ameliorates brain IRI. Using the intraluminal filament or suture model of middle cerebral artery occlusion (MCAO), it was found that pazopanib treatment showed less infarct size and improved neurological deficit score when given i.v. 0.5 hrs after reperfusion. As further demonstrated herein, pazopanib increases ROS production in myeloid cells and ameliorates acute lung injury. It was found that pazopanib enhances pulmonary vasculature integrity and promotes lung epithelial cell survival and proliferation, leading to increased pulmonary barrier function and resistance to ALI. Furthermore, pazopanib was found to reduce ALI mortality and to reduce edema. Accordingly, pazopanib was shown to recapitulate the effects of MAP3K2/3 deficiency in 2 mouse ALI models, that is, reduction of pulmonary permeability and interstitial edema and increased survival. Furthermore, in a coronavirus-induced mouse lung injury model, murine hepatitis virus strain 1 (MHV-1), treatment with pazopanib provided significant reduction in pulmonary permeability.

No drugs have previously demonstrated any significant improvement in survival for patients with ALI or ARDS, and currently there are no drugs approved for treatment of lung injury (ALI or ARDS) in SARS CoV-2 infected patients. Furthermore, the MoA (mechanism of action) for pazopanib in ALI is unique and distinct from those of other drugs that have been evaluated clinically in patients with ALI/ARDS to date. It is also distinct from other “‘immunosuppressive’” agents under investigation for COVID-19, which target different aspects of the immune response. Use of immunosuppressive agents requires a balance between suppression of pathologic immune responses and conservation of immune-mediated viral clearance. In one aspect, based on the newly discovered potential mechanism for pazopanib in ALI/ARDS, immune suppression is not anticipated, and thus, this agent offers a more advantageous alternative treatment for patients suffering from a coronavirus infection such as COVID-19.

The present disclosure provides a method of treating, ameliorating, and/or preventing IRI, lung injury related to a coronavirus infection, ALI, and/or ARDS in a subject, comprising administering to the subject a therapeutically effective amount of pazopanib, or a salt or solvate thereof. In certain embodiments, the pazopanib, or salt or solvate thereof, is administered to the subject after the reperfusion takes place.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound of the disclosure or salt thereof along with a compound that may also treat the disorders or diseases contemplated within the disclosure. In certain embodiments, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of one or more signs, symptoms, or causes of a disease, or any other desired alteration of a biological system. An appropriate therapeutic amount in any individual case may be determined by one of ordinary skill in the art using routine experimentation.

“Instructional material,” as that term is used herein, includes a publication, a recording, a diagram, or any other medium of expression that can be used to communicate the usefulness of the composition and/or compound of the disclosure in a kit. The instructional material of the kit may, for example, be affixed to a container that contains the compound and/or composition of the disclosure or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

As used herein, the term “pazopanib” refers to 5-((4-((2,3-dimethyl-2H-indazol-6-yl)(methyl)amino)pyrimidin-2-yl)amino)-2-methylbenzenesulfonamide, or a salt, tautomer, and/or solvate thereof:

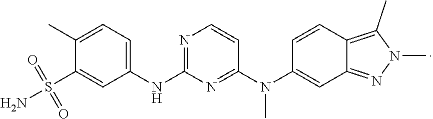 [SMILES: Cc1ccc(Nc2nccc(N(C)c3ccc4c(C)n(C)nc4c3)n2)cc1S(N)(=O)=O]

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

The term “prevent,” “preventing,” or “prevention” as used herein means avoiding or delaying the onset of one or more symptoms associated with a disease or condition in a subject that has not developed such symptoms at the time the administering of an agent or compound commences.

As used herein, the term “reperfusion injury” or “ischemia-reperfusion injury” or “IRI” or “reoxygenation injury” is the tissue damage caused when blood supply returns to tissues after a period of ischemia or lack of oxygen (anoxia or hypoxia). The absence of oxygen and nutrients from blood during the ischemic period creates a condition in which the restoration of circulation results in inflammation and oxidative damage through the induction of oxidative stress rather than (or along with) restoration of normal function. Reperfusion of ischemic tissues is often associated with microvascular injury, particularly due to increased permeability of capillaries and arterioles that lead to an increase of diffusion and fluid filtration across the tissues. Reperfusion injury plays a major part in the biochemistry of hypoxic brain injury in stroke. Similar failure processes are involved in brain failure following reversal of cardiac arrest. Repeated bouts of ischemia and reperfusion injury also are thought to be a factor leading to the formation and failure to heal of chronic wounds such as pressure sores and diabetic foot ulcer. Continuous pressure limits blood supply and causes ischemia, and the inflammation occurs during reperfusion. As this process is repeated, it eventually damages tissue enough to cause a wound. Further, reperfusion injury is a common complication of transplantation surgery (such as but not limited to liver, lung, heart, and kidney).

As used herein, the term “ROS” refers to reactive oxygen species. Non-limiting examples of ROS are peroxide, superoxide, hydroxyl radical, and/or singlet oxygen.

The term “salt” embraces addition salts of free acids and/or basis that are useful within the methods of the disclosure. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds and/or compositions useful within the methods of the disclosure. Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, 2-hydroxyethanesulfonic, p-toluenesulfonic, trifluoromethanesulfonic, sulfanilic, cyclohexylaminosulfonic, stearic, alginic, (3-hydroxybutyric, salicylic, galactaric and galacturonic acid. Suitable pharmaceutically acceptable base addition salts of compounds and/or compositions of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (also known as N-methylglucamine) and procaine. All of these salts may be prepared from the corresponding compound by reacting, for example, the appropriate acid or base with the compound and/or composition.

As used herein, a “solvate” of a compound refers to the entity formed by association of the compound with one or more solvent molecules. Solvates include water, ether (e.g., tetrahydrofuran, methyl tert-butyl ether) or alcohol (e.g., ethanol) solvates, acetates and the like. In certain embodiments, the compounds described herein exist in solvated forms with solvents such as water, and ethanol. In other embodiments, the compounds described herein exist in unsolvated form.

By the term “specifically bind” or “specifically binds,” as used herein, is meant that a first molecule preferentially binds to a second molecule (e.g., a particular receptor or enzyme), but does not necessarily bind only to that second molecule.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein and/or one or more symptoms of a condition contemplated herein, with the purpose to cure, heal, alleviate, relieve, alter, remedy, ameliorate, improve, or affect a condition contemplated herein and/or one or more symptoms of a condition contemplated herein. Such treatments may be specifically tailored or modified, based on knowledge obtained from the field of pharmacogenomics.

The following non-limiting abbreviations are used herein: MAP3K2 or MEKK2, mitogen-activated protein kinase kinase kinase 2; MAP3K3 or MEKK3, mitogen-activated protein kinase kinase kinase 3; MEK, mitogen-activated protein kinase kinase; MEKK, MEK kinase; RBC, red blood cell; ROS, reactive oxygen species.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### Compounds and Compositions

In certain embodiments, pazopanib, or a salt or solvate thereof, is useful within the methods of the disclosure. In other embodiments, compounds and/or compositions useful within the disclosure are recited in U.S. Pat. Nos. 7,105,530; 7,262,203; 7,858,626; and 8,114,885; all of which are incorporated herein in their entireties by reference. Compositions comprising pazopanib, or a salt or solvate thereof, are also contemplated within the disclosure.

### Methods

Method of Preventing, Ameliorating, and/or Treating Reperfusion Injury, Ischemia-Reperfusion Injury, and/or Reoxygenation Injury

The disclosure includes a method of preventing, ameliorating, and/or treating reperfusion injury, ischemia-reperfusion injury, and/or reoxygenation injury in a subject in need thereof. The disclosure includes a method of preventing, ameliorating, and/or treating ischemia-reperfusion injury in a subject suffering from ischemic stroke. The disclosure includes a method of preventing, ameliorating, and/or treating ischemia-reperfusion injury in a subject not suffering from ischemic stroke.

In certain embodiments, the method comprises administering to the subject therapeutically effective amounts of pazopanib, and/or a salt and/or solvate thereof. In other embodiments, the administration route is oral. In other embodiments, the administration route is parenteral. In yet other embodiments, the administration route is selected from the group consisting of oral, parenteral, nasal, inhalational, intratracheal, intrapulmonary, and intrabronchial.

In certain embodiments, the compositions of the disclosure are administered to the subject about three times a day, about twice a day, about once a day, about every other day, about every third day, about every fourth day, about every fifth day, about every sixth day and/or about once a week.

In certain embodiments, the compositions of the disclosure are administered to the subject after perfusion has taken place.

In certain embodiments, the dose of pazopanib, or a salt or solvate thereof, required to treat IRI in a subject is lower than the dose of pazopanib, or a salt or solvate thereof, required to treat cancer (such as but not limited to advanced renal cell carcinoma) in a subject orally. In other embodiments, the dose used within the methods of the disclosure is about 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95 or 1:100 that of the oral dose required to treat cancer, in terms of mass of pazopanib, or a salt or solvate thereof, per subject's weight. In yet other embodiments, the dose of drug is about 5-200 mg/day.

In certain embodiments, administration of the compound and/or composition to the subject does not cause significant adverse reactions, side effects and/or toxicities that are associated with administration of the compound and/or composition to treat cancer. Non-limiting examples of adverse reactions, side effects and/or toxicities include, but are not limited to hepatotoxicity (which may be evidenced and/or detected by increases in serum transaminase levels and bilirubin), prolonged QT intervals and torsades de pointes, hemorrhagic events, decrease or hampering of coagulation, arterial thrombotic events, gastrointestinal perforation or fistula, hypertension, hypothyroidism, proteinuria, diarrhea, hair color changes (depigmentation), nausea, anorexia, and vomiting.

In certain embodiments, the subject is undergoing treatment in an intensive care unit (ICU). In other embodiments, the subject is undergoing treatment in an emergency room (ER). In yet other embodiments, the subject is on a ventilator.

In certain embodiments, the subject is further administered at least one additional agent that treats, prevents, ameliorates, and/or reduces one or more symptoms of the IRI.

In certain embodiments, the subject is a mammal. In other embodiments, the mammal is a human.

The disclosure further provides a method of evaluating efficacy of a drug in treating IRI. In certain embodiments, the method comprises contacting a neutrophil with the drug and measuring neutrophil ROS production levels after the contacting. If the neutrophil ROS production levels increase after the contacting, the drug is efficacious in treating IRI.

The disclosure further provides a method of evaluating efficacy of a drug in treating a subject suffering from IRI. In certain embodiments, the method comprises measuring neutrophil ROS production levels in the subject after being administered the drug If the neutrophil ROS production levels in the subject after being administered the drug are higher than the neutrophil ROS production levels in the subject before being administered the drug, the drug is efficacious in treating IRI in the subject.

Method of Preventing, Ameliorating, and/or Treating Lung Injury

In another aspect, the present disclosure relates to a method of preventing, ameliorating, and/or treating lung injury related to a coronavirus infection or acute lung injury in a subject in need thereof. In certain embodiments, the method comprises administering to the subject therapeutically effective amounts of pazopanib, or a salt or solvate thereof.

In certain embodiments, the lung injury related to a coronavirus infection has progressed to acute lung injury. In certain embodiments, the lung injury related to a coronavirus infection has not progressed to ALI. In certain embodiments, the coronavirus infection is COVID-19. In certain embodiments, the acute lung injury is ARDS. In certain embodiments, the ALI/ARDS is lipopolysaccharide (LPS)-induced ALI/ARDS. In certain embodiments, the ALI is aspiration-induced ALI/ARDS. In certain embodiments the subject afflicted with aspiration-induced ALI/ARDS is a subject with a disturbed consciousness (such as, but not limited to, drug overdose, seizures, cerebrovascular accident, sedation, anesthetic procedures) or a frail older adult subject. In certain embodiments, the lung injury is ALI/ARDS caused by ischemia reperfusion. In certain embodiments, the method treats, ameliorates, and/or prevents ALI/ARDS caused by ischemia reperfusion injury associated with lung transplantation. In certain embodiments, the acute lung injury is ARDS caused by a viral and/or bacterial infection. In certain embodiments, the ALI/ARDS is associated with a coronavirus infection. In certain embodiments, the coronavirus infection is COVID-19.

In certain embodiments, the pazopanib salt is pazopanib hydrochloride. In certain embodiments, the pazopanib or salt or solvate thereof is administered as a composition or formulation comprising any additional ingredients known to a person of skill in the art. In certain embodiments, the composition/formulation comprising pazopanib or salt or solvate thereof comprises hydroxypropyl betadex (HPB). In certain embodiments, the composition/formulation comprising pazopanib or salt or solvate thereof is an intravenous composition comprising pazopanib hydrochloride, HPB, and water for injection.

The pazopanib or salt or solvate thereof can be administered in any fashion known to a person of skill in the art. In certain embodiments, the administration route is oral. In certain embodiments, the administration route is nasal. In certain embodiments, the administration route is intravenous. In other embodiments, the administration route is selected from the group consisting of oral, parenteral (such as, but not limited to, intravenous), nasal, inhalational, intratracheal, intrapulmonary, and intrabronchial.

In certain embodiments, the compounds and/or compositions of the disclosure are administered to the subject before a lung injury related to a coronavirus infection and/or ALI/ARDS occurs. In certain other embodiments, the compounds and/or compositions of the disclosure are administered to the subject after a lung injury related to a coronavirus infection and/or ALI/ARDS occurs. In certain embodiments, the compositions of the disclosure are administered to the subject about three times a day, about twice a day, about once a day, about every other day, about every third day, about every fourth day, about every fifth day, about every sixth day and/or about once a week. In certain other embodiments, the compounds and/or compositions of the disclosure are administered for a brief period of time before an occurrence that could result in a lung injury related to a coronavirus infection and/or ALI/ARDS such as sedation, an anesthetic procedure, or lung transplantation. In certain embodiments, the brief period of time comprises between about a month to about a day before an occurrence that could result in a lung injury related to a coronavirus infection and/or ALI/ARDS. In certain embodiments, the compounds and/or compositions of the disclosure are administered to the subject the day of an occurrence that could result in a lung injury related to a coronavirus infection and/or ALI/ARDS, wherein the compounds and/or compositions may be administered any time that day, up to immediately before an occurrence that could result in a lung injury related to a coronavirus infection and/or ALI/ARDS.

In certain embodiments, the dose of pazopanib, or a salt or solvate thereof, required to treat a lung injury related to a coronavirus infection and/or ALI/ARDS in a subject is lower than the dose of pazopanib, or a salt or solvate thereof, required to treat cancer (such as but not limited to advanced renal cell carcinoma) in a subject orally. In other embodiments, the dose used within the methods of the disclosure is about 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95 or 1:100 that of the oral dose required to treat cancer, in terms of mass of pazopanib, or a salt or solvate thereof, per subject's weight. In yet other embodiments, the dose of drug is about 5-500 mg/day. In certain embodiments, the dose of drug is about 5-450 mg/day. In certain embodiments, the dose of drug is about 5-400 mg/day. In certain embodiments, the dose of drug is about 5-350 mg/day. In certain embodiments, the dose of drug is about 5-300 mg/day. In certain embodiments, the dose of drug is about 5 mg/day, 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 155 mg/day, 160 mg/day, 165 mg/day, 170 mg/day, 175 mg/day, 180 mg/day, 185 mg/day, 190 mg/day, 195 mg/day, 200 mg/day, 205 mg/day, 210 mg/day, 215 mg/day, 220 mg/day, 225 mg/day, 230 mg/day, 235 mg/day, 240 mg/day, 245 mg/day, 250 mg/day, 255 mg/day, 260 mg/day, 265 mg/day, 270 mg/day, 275 mg/day, 280 mg/day, 285 mg/day, 290 mg/day, 295 mg/day, or 300 mg/day. In certain embodiments, the dose of drug is equal to or greater than about 5 mg/day, 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 155 mg/day, 160 mg/day, 165 mg/day, 170 mg/day, 175 mg/day, 180 mg/day, 185 mg/day, 190 mg/day, 195 mg/day, 200 mg/day, 205 mg/day, 210 mg/day, 215 mg/day, 220 mg/day, 225 mg/day, 230 mg/day, 235 mg/day, 240 mg/day, 245 mg/day, 250 mg/day, 255 mg/day, 260 mg/day, 265 mg/day, 270 mg/day, 275 mg/day, 280 mg/day, 285 mg/day, 290 mg/day, 295 mg/day, or 300 mg/day. In certain embodiments, the dose of drug is equal to or lower than about 5 mg/day, 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 155 mg/day, 160 mg/day, 165 mg/day, 170 mg/day, 175 mg/day, 180 mg/day, 185 mg/day, 190 mg/day, 195 mg/day, 200 mg/day, 205 mg/day, 210 mg/day, 215 mg/day, 220 mg/day, 225 mg/day, 230 mg/day, 235 mg/day, 240 mg/day, 245 mg/day, 250 mg/day, 255 mg/day, 260 mg/day, 265 mg/day, 270 mg/day, 275 mg/day, 280 mg/day, 285 mg/day, 290 mg/day, 295 mg/day, or 300 mg/day. In certain embodiments, the dose of drug is about 5-250 mg/day. In certain embodiments, the dose of drug is about 5-200 mg/day. In certain embodiments, the dose of drug is about 5-150 mg/day. In certain embodiments, the dose of drug is about 5-100 mg/day. In certain embodiments, the dose of drug is about 200 mg/day. In certain embodiments, the intranasal or oral dose of drug is about 200 mg/day. In certain embodiments, the dose of drug is about 80 mg/day. In certain embodiments, the intravenous dose of drug is about 80 mg/day. In certain embodiments, the intravenous dose of drug is about 80 mg/day of a pazopanib hydrochloride composition/formulation further comprising HPB and water for injection.

In certain embodiments, administration of the compound and/or composition to the subject does not cause significant adverse reactions, side effects and/or toxicities that are associated with administration of the compound and/or composition to treat cancer. Non-limiting examples of adverse reactions, side effects and/or toxicities include, but are not limited to hepatotoxicity (which may be evidenced and/or detected by increases in serum transaminase levels and bilirubin), prolonged QT intervals and torsades de pointes, hemorrhagic events, decrease or hampering of coagulation, arterial thrombotic events, gastrointestinal perforation or fistula, hypertension, hypothyroidism, proteinuria, diarrhea, hair color changes (depigmentation), nausea, anorexia, and/or vomiting.

In certain embodiments, the subject is undergoing treatment in an intensive care unit (ICU). In other embodiments, the subject is undergoing treatment in an emergency room (ER). In yet other embodiments, the subject is on a ventilator. In certain embodiments, the subject is undergoing treatment which comprises sedation or an anesthetic procedure. In certain embodiments, the subject is undergoing a lung transplant. In certain embodiments, the subject is undergoing treatment for a coronavirus infection. In certain embodiments, the subject is undergoing treatment for COVID-19.

In certain embodiments, the subject is further administered at least one additional agent that treats, prevents, ameliorates, and/or reduces one or more symptoms of the ALI/ARDS. Exemplary agents include, but are not limited to, a glucocorticoid, a surfactant, N-acetylcysteine, inhaled nitric oxide, liposomal PGE 1, a phosphodiesterase inhibitor (e.g. lisofylline, pentoxifylline), salbutamol IV, procysteine, activated protein C, inhaled albuterol, an antifungal agent, a diuretic, or a combination thereof. In certain embodiments, the subject is provided a treatment that treats, prevents, ameliorates, and/or reduces one or more symptoms of the ALI/ARDS. Exemplary treatments include, but are not limited to, ventilator support, prone positioning, extracorporeal membrane oxygenation, or a combination thereof.

In certain embodiments, the subject is further administered at least one additional agent treatment and/or therapy for a coronavirus infection. Treatment and/or therapy can include over-the-counter medicines, e.g., acetaminophen, to relieve symptoms; mechanical ventilation; anti-virals; and plasma therapy. Exemplary antiviral drugs include, but are not limited to, abacavir, acyclovir, adefovir, amantadine, ampligen, amprenavir, arbidol umifenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, pleconaril, podophyllotoxin, raltegravir, remdesivir, ribavirin, rilpivirine, rimantadine, ritonavir, saquinavir, simeprevir, sofosbuvir, stavudine, taribavirin, telaprevir, telbivudine, tenofovir alafenamide, tenofovir disoproxil, tenofovir, tipranavir, trifluridine, trizivir, tromantadine, truvada, umifenovir, valaciclovir, valganciclovir, vicriviroc, vidarabine, zalcitabine, zanamivir, zidovudine, and combinations thereof. In certain embodiments, the treatment and/or therapy comprises a pharmaceutically active compound that aids in the treatment, amelioration, and/or prevention of a coronavirus infection, such as SARS-CoV-2. Exemplary compounds believed to aid in the treatment, amelioration, and/or prevention of a coronavirus infection include, but are not limited to, remdesivir, dexamethasone, hydroxychloroquine, chloroquine, azithromycin, tocilizumab, acalabrutinib, tofacitinib, ruxolitinib, baricitnib, anakinra, canakinumab, apremilast, marillimumab, sarilumab, lopinavir, ritonavir, oseltamivir, favipiravir, umifenovir, galidesivir, colchicine, ivermectin, vitamin D, and combinations thereof. In certain embodiments, a subject who is determined to be infected with a coronavirus (e.g, SARS-CoV-2) or a subject diagnosed with an infection or disease caused by a coronavirus (e.g., COVID-19) is quarantined or asked to self-quarantine.

In certain embodiments, the subject is a mammal. In other embodiments, the mammal is a human.

The disclosure further provides a method of evaluating efficacy of a drug in treating lung injury related to a coronavirus infection and/or ALI/ARDS. In certain embodiments, the method comprises contacting a neutrophil with the drug and measuring neutrophil ROS production levels after the contacting. If the neutrophil ROS production levels increase after the contacting, the drug is efficacious in treating lung injury related to a coronavirus infection and/or ALI/ARDS.

The disclosure further provides a method of evaluating efficacy of a drug in treating a subject suffering from coronavirus related lung injury and/or ALI/ARDS. In certain embodiments, the method comprises measuring neutrophil ROS production levels in the subject after being administered the drug. If the neutrophil ROS production levels in the subject after being administered the drug are higher than the neutrophil ROS production levels in the subject before being administered the drug, the drug is efficacious in treating coronavirus related lung injury and/or ALI/ARDS in the subject. In other embodiments, the method comprises measuring the level of H₂O₂ in the lungs of the subject after being administered the drug. If the levels of H₂O₂ in the lungs of the subject after being administered the drug are higher than H₂O₂ in the lungs of the subject before being administered the drug, the drug is efficacious in treating coronavirus related lung injury and/or ALI/ARDS in the subject.

Although not wishing to be limited by theory, it is believed that administering to the subject therapeutically effective amounts of pazopanib, or a salt or solvate thereof results in the inhibition of MEK kinases MAP3K2 and MAP3K3, wherein the inhibition of MAP3K2 and MAP3K3 leads to increased ROS from neutrophils. It is hypothesized that ROS is converted to H₂O₂ in the lungs, which stimulates AKT phosphorylation in endothelial cells, leading to stronger vessel barrier integrity, the prevention of capillary leakage, and clearing of alveolar fluid in the lungs. It is also believed that low concentrations of H₂O₂ enhance trans-endothelial electrical resistance of lung endothelial cells and stimulate AKT phosphorylation in these cells. Therefore, it is hypothesized that administering to the subject therapeutically effective amounts of pazopanib, or a salt or solvate thereof results in enhanced pulmonary vasculature integrity and promotes lung epithelial cell survival and proliferation, leading to increased pulmonary barrier function and resistance to coronavirus infection and/or ALI/ARDS. In certain embodiments, the coronavirus infection is COVID-19.

### Kits

The disclosure includes a kit comprising pazopanib, and/or a salt and/or solvate thereof, an applicator, and an instructional material for use thereof. The instructional material included in the kit comprises instructions for preventing, ameliorating, and/or treating IRI, coronavirus related lung injury, ALI/ARDS, or any other disease or disorder contemplated within the disclosure. The instructional material recites the amount of, and frequency with which, the pazopanib, and/or a salt and/or solvate thereof, should be administered to the subject. In other embodiments, the kit further comprises at least one additional agent that treats, ameliorates, prevents, and/or reduces one or more symptoms of IRI, coronavirus infection, and/or ALI/ARDS. In certain embodiments, the kit further comprises instructions for providing the subject with a treatment that is believed to treat, ameliorate, prevent, and/or reduce one or moresymptoms of the ALI/ARDS and/or coronavirus infection. Exemplary treatments are described elsewhere herein.

### Combination Therapies

In certain embodiments, the compounds of the disclosure are useful in the methods of the disclosure in combination with at least one additional compound and/or therapy useful for treating, ameliorating, and/or preventing IRI, coronavirus infection, or ALI/ARDS. This additional compound may comprise compounds identified herein or compounds, e.g., commercially available compounds, known to treat, ameliorate, prevent, and/or reduce one or more symptoms of IRI, coronavirus infection, and/or ALI/ARDS.

Non-limiting examples of additional therapies contemplated within the disclosure include anti-inflammatory steroids or non-steroid drugs.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated in the disclosure. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

Administration of the compositions of the present disclosure to a patient, preferably a mammal, more preferably a human, may be carried out using known procedures, at dosages and for periods of time effective to treat a disease or disorder contemplated in the disclosure. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the disclosure. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

The therapeutically effective amount or dose of a compound of the present disclosure depends on the age, sex and weight of the patient, the current medical condition of the patient and the progression of a disease or disorder contemplated in the disclosure.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In certain embodiments, the dose of a compound of the disclosure is from about 1 mg and about 2,500 mg. In certain embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in certain embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the compositions of the disclosure are administered to the patient in dosages that range from one to five times per day or more. In certain embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of one or more symptoms.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, ameliorate, and/r reduce one or more symptoms of a disease or disorder contemplated in the disclosure.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the disclosure include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and/or treatment conditions with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

### Example 1: Pazopanib Ameliorates Cerebral Ischemia-Reperfusion Injury

### Methods:

### Intraluminal Middle Cerebral Artery (MCA) Occlusion:

Transient focal ischemia was produced by intraluminal middle cerebral artery (MCA) occlusion with a nylon filament. This is one of the most widely used in stroke research. This model, to some extent, simulates the restoration of blood flow after spontaneous or therapeutic intervention (e.g., tPA administration) to lyse a thromboembolic clot in humans.

Mice were anesthetized with 2.5% isoflurane in a 70% N₂O/30% O₂ mixture. After midline neck incision, the left common carotid artery, external carotid artery, and internal carotid artery were carefully separated. The proximal left common carotid artery and the external carotid artery were ligated. A silicone-rubber coated nylon monofilament (0.23 mm, Yushun Bio) was introduced through a small arteriotomy of the common carotid artery into the distal internal carotid artery and was advanced 8-9 mm distal to the origin of the MCA, until the MCA was occluded. The suture was withdrawn from the carotid artery under anesthesia 1 h after insertion to enable reperfusion. Then, the wound was closed. Mice were maintained in an air-conditioned room at 25° C. during the reperfusion period of 24 h.

### Evaluation of Neurological Deficit Score:

Neurological deficits of the mice that had undergone stroke surgery were measured on a scale of 0-4. After 1 h occlusion and 24 h reperfusion, the animals were scored for neurological damage as follows: 0=normal spontaneous movement; 1=failure to extend forelimb; 2=circling to affected side; 3=partial paralysis on affected side; 4=no spontaneous motor activity.

### Determination of Infarct Size:

After 24 h reperfusion, mice were killed with CO₂. The brains were immediately removed and sectioned into five coronal slices. The brain slices were incubated in 2% 2,3,5-triphenyltetrazolium chloride monohydrate (TTC) at 37° C. for 15 min, followed by 4% paraformaldehyde overnight. The brain slices were photographed and the area of ischemic damage was measured by an imaging analysis system (NIH Image). The percentage of brain infarct was calculated with the following formula: %=infarct volume/total brain volume.

### Drug Preparation and Administration:

Pazopanib was dissolved in HP-beta-CD (2-Hydroxypropyl)-β-cyclodextrin) at 8.6 mg/ml as the stock solution. It was diluted in saline at 1.2 mg/ml. 50 μl/mice were administered via retro-orbital IV injection.

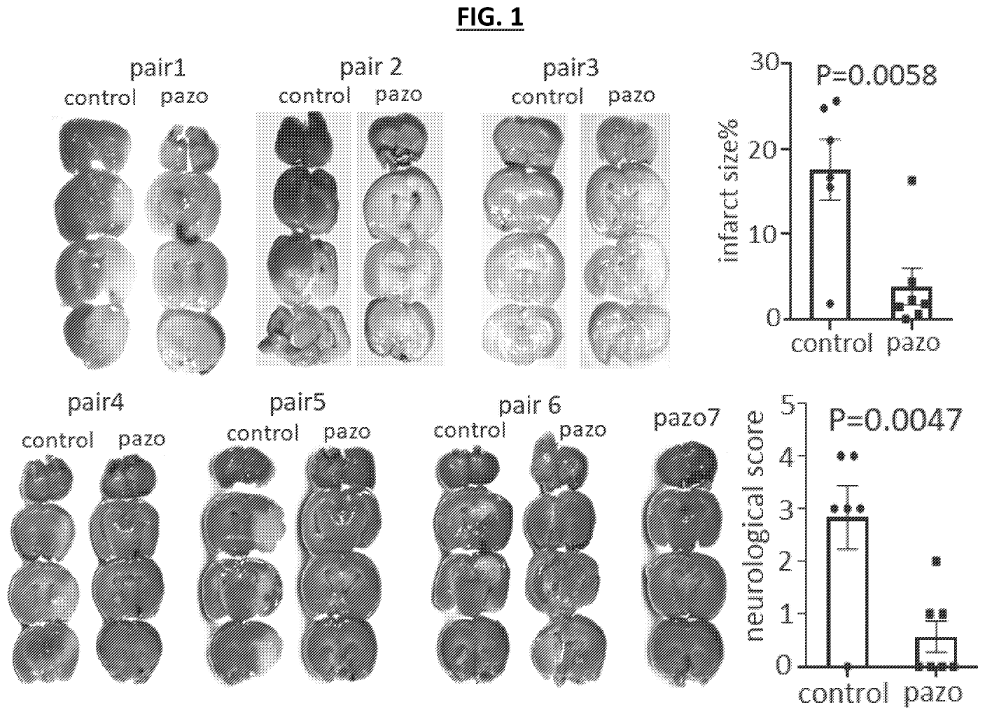

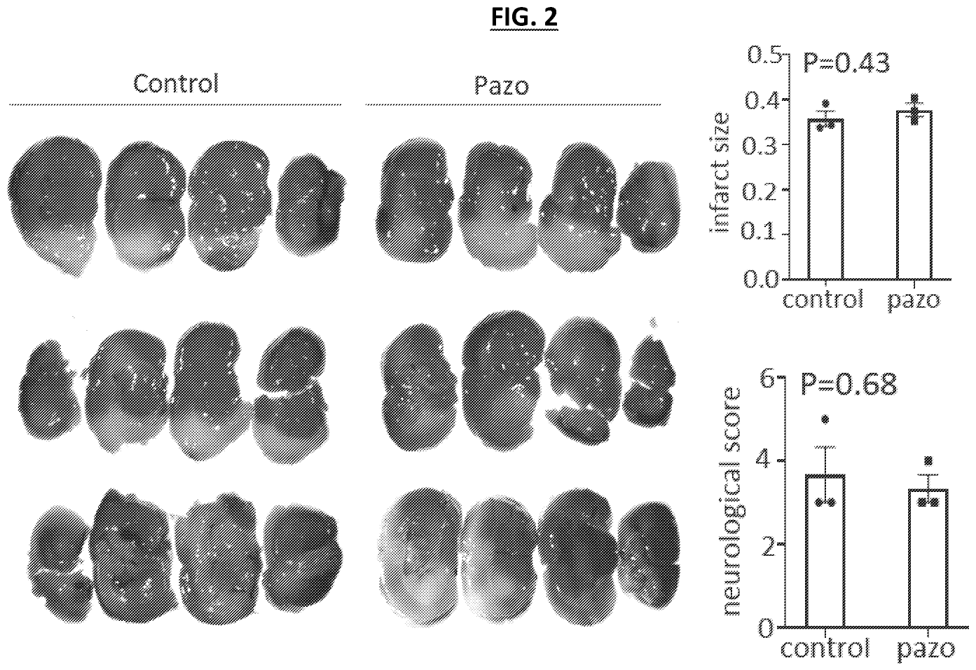

Effects of pazopanib were tested on cerebral ischemia-reperfusion injury. To test the therapeutic impact, pazopanib was given intravenously. Two time point were selected, (1) during the acute phase of ischemic stroke and (2) 0.5 hr after reperfusion. Pazopanib treatment showed less infarct size when given 0.5 hrs after reperfusion (FIG. 1). If the drug was given during ischemic phase, there was no improvement in the infarct size of the brain or neurological score (FIG. 2).

### Example 2: Pazopanib Ameliorates Acute Lung Injuries Via Inhibition of MAP3K2 and 3

### Materials and Methods:

### Materials

The following reagents were purchased from Sigma: N-Formyl-L-methionyl-L-leucyl-L-phenylalanine (fMLP), Phorbol 12-myristate 13-acetate (PMA), Lipopolysaccharide (LPS), Lysolecithin, Paraformaldehyde (PFA), FITC Albumin, Horse Reddish Peroxidase (HRP), Isoluminol. Percoll was purchased from GE Healthcare (Uppsala, Sweden), Bovine Serum Albumin (BSA) from American Bio (Natick, Mass.), GMCSF from Peprotech, Lipofectamine kit and Cell trace dyes from Thermo Fisher. The following material were purchased from GIBCO: Dulbecco's Modified Eagle Medium (DMEM), Hanks Balanced Salt Solution (HBSS), Phosphate Buffered Saline (PBS).

The commercial antibodies used in the study are: GST antibody (2624, Cell signaling), His antibody (2366, Cell Signaling), HA antibody (MMS-101R, Covance), Myc antibody (MMS-150R, Covance), anti-phospho-AKT antibody (4060 and 2965, Cell Signaling), anti-AKT antibody (9272, Cell Signaling), anti-MEKK2 (19607, Cell Signaling), anti-MEKK3 antibody (5727, Cell Signaling), anti-p47^{phox} antibody (17875, Santa Cruz), anti-CD31 antibody (102502, BioLegend), anti-α-smooth muscle actin antibody (ab8211, Abcam), anti-ABCA3 antibody (ab24751, Abcam), anti-podoplanin antibody (AF3244-SP, R&D), anti-4 Hydroxynonenal antibody (ab46545, Abcam), anti-Cleaved caspase 3 antibody (9661, Cell signaling), anti-Ki67 antibody (9129, Cell signaling), anti Rac1 antibody (ab33186, Abcam), anti-active Rac1 antibody (26903, NewEast), and anti-β-actin antibody (4967, Cell Signaling). The rabbit polyclonal anti-S208 p47^{phox} was made from a synthetic peptide (KRGWVPApSYLEPLD; SEQ ID NO: 1) at Abiocode.

Protein A/g PLUS-agarose beads were purchased from Santa Cruz Biotechnology (Santa Cruz, Calif.). ELISA kits for cytokine measurements were purchased from eBioscience (San Diego, Calif.). The cDNAs for MAP3K3 and p67^{phox} were acquired from ADDGENE, and cDNAs for p47^{phox} and gp91^{phox} from Open Biosystems.

HEK293 and Cos-7 cells were purchased from ATCC. Cells have been routinely tested for mycoplasma and they were negative.

### Mice

The Map3k2^{-/-} mice were previously described in Guo, et al., 2002, Mol Cell Biol 22:5761-5768, whereas the Map3k3^{fl/fl} mice were described in Wang, et al., 2009, J Immunol 182:3597-3608. Both Map3k2^{-/-} and Map3k3^{fl/fl} were backcrossed into the C57Bl/6N background. The p47^{phox}-deficient mice (B6N.129S2-Ncf1^{tm1Sh1}/J) were obtained from JAX together with WT control mice for all experiments involving p47^{phox}-deficient mice. The myeloid-specific MAP3K3 KO, MAP3K2 KO and DKO mice were generated by intercrossing Map3k3^{fl/fl} and/or Map3k2^{fl/fl} mice with the Lyz-Cre mice. These mice are all in C57Bl/6N backgrounds. The p47^{phox} S208A knock-in mouse line was generated by CRISPR/Cas in C57Bl/6N background by Cyagen Biosciences.

### Bone Marrow Transplantation

Bone marrows from littermates of WT and mutant mice were transplanted into wildtype recipient C57Bl/6N mice purchased from Envigo (East Millstone, N.J.), which had been subjected to 1000 cGy X-Ray irradiation. Eight weeks later, the transplanted mice were used for experiments.

### Neutrophil Preparation and Transfection

Mouse bone marrow neutrophils were isolated from long bones. After lysis of red blood cells (RBCs) with ACK buffer (155 mM NH₄Cl, 10 mM KHCO₃ and 127 μM EDTA), bone marrow cells were separated on a discontinuous Percoll gradient composed of 81%, 62%, and 45% Percoll. Neutrophils were collected at the interphase between 81% and 62% Percoll, washed in HBSS, and used for assays.

For neutrophil transfection, neutrophils (3×10⁶ cells/100 μl) were mixed with 1.6 μg of DNA in the supplied nucleofection solution and electroporated using a Nucleofector device (Lonza, Switzerland). The cells were then cultured in the medium (RPMI 1640, 10% FBS (V/V), GMCSF 25 ng/ml) at 37° C. in humidified air with 5% CO₂ for 8-24 hours before assays.

### Dunn Chamber Chemotaxis Assay

The chemotaxis assay using a Dunn chamber was carried out as previously described. Wildtype and mutant neutrophils were analyzed simultaneously by labeling the cells with different tracing dyes. The labeled group was alternated in the study to completely eliminate the possibility of any influence from the dye. Time-lapse image series were acquired at 30-second intervals for 30 mins and were analyzed using the MetaMorph image analysis software as previously described. Two parameters are obtained to quantify neutrophil chemotaxis: average directional errors and motility. The average directional error measures the angle between the cell migration direction and the gradient direction and reflects how well a cell follows the gradient. Motility is cell migration speed.

### Integrin Expression Assay

Bone marrow-derived neutrophils were resuspended in flow cytometry buffer (PBS with 1% BSA), stimulated with fMLP (1 μM) for indicated durations, fixed with 4% PFA, and then stained with FITC labeled anti LFA-1 or anti Mac-1. Samples were analyzed by BD LSR II flow cytometer.

### ICAM-I Binding Assay

The assay was carried out as previously described. The ICAM-1-Fc-F(ab′)2 complexes was generated by incubating Cy5-conjugated AffiniPure goat anti-human Fcγ fragment-specific IgG F(ab′)2 fragments (Jackson Immunobiology) and ICAM-1-Fc (100 μg/ml, R&D) at 4° C. for 30 min in PBS. Neutrophils, which were resuspended at 0.5×10⁶ cells/ml in PBS containing 0.5% BSA, 0.5 mM Mg²⁺ and 0.9 mM Ca²⁺, were mixed with the ICAM-1-Fc-F(ab′)2 complexes in the presence or absence of fMLP (1 μM) for 5 min. The reactions were terminated by adding 4% paraformaldehyde. After 5 min, fixation was stopped by adding 3 ml ice-cold FACS buffer. Cells were pelleted, resuspended in 300 μl of FACS buffer, and analyzed on a flow cytometer.

Neutrophil Infiltration into Inflamed Peritoneum and Flow Chamber Adhesion Assay

For the peritonitis infiltration model, purified wild type and mutant neutrophils were labeled with 2.5 μM CFSE [5-(and -6)-carboxyfluorescein diacetate succinimidyl esters] and 2.5 μM Far-Red DDAO SE, respectively, and vice versa. The WT and mutant cells with different fluorescence labels were mixed at a 1:1 ratio and injected into retro-orbital venous sinus of wildtype littermates, which were injected with 2 ml of 3% Thioglycolate (TG) two hours earlier. The mice were euthanized one and half hour later. Cells in their peritonea were collected and analyzed by cell counting and flow cytometry. The data presented are the combination of the experiments with reciprocal fluorescence labeling.

To examine neutrophil adherence to endothelial cells under shear stress, mouse endothelial cells were cultured to confluency on 10 μg/ml fibronectin coated coverslips and treated with 50 ng/ml TNFα for 4 hours. The coverslips containing the endothelial cell layer were washed with PBS and placed in a flow chamber apparatus (GlycoTech). The WT and mutant cells labeled different fluorescence labels as described above were mixed at a 1:1 ratio and flowed into the chamber at a shear flow rate of 1 dyn/cm². The adherent cells were then examined and counted under a fluorescence microscope.

### ROS Release Assay

For measurement of extracellular ROS release, isolated neutrophils were placed in the reaction buffer (0.25% BSA in HBSS with Ca²⁺ and Mg²⁺, 10 mM Isoluminol, 100 u/ml HRP) and stimulated with fMLP or PMA. For measurement of total ROS production, neutrophils were incubated with the reaction buffer (0.25% BSA in HBSS with Ca²⁺ and Mg²⁺, 10 mM Luminol, 100 u/ml HRP), followed with stimulations. Chemiluminescence was read continuously in a plate reader (Perkin Elmer). For reconstituted ROS production system in COS-7 cells, PMA (2 μM) was used for stimulation.

Superoxide production in mouse primary neutrophils was also measured by the cytochrome C assay. Briefly, cytochrome C (100 μM, Sigma C2506) was added to the mouse primary neutrophil suspension. Then, 90 μl aliquots (1×10⁶ cells) were transferred to individual wells of a 96-well plate and a basal reading was performed at 540 nm (isosbestic point of cytochrome C) and 550 nm (SpectraMax iD3; Molecular Devices). The oxidative burst was subsequently initiated by the addition of 10 μl fMLP (final concentration 4 μM). The absorbance at 540 nm and 550 nm were recorded every 14 seconds for 30 min. Signals were calculated by normalization of the signals obtained at 540 nm.

### Neutrophil Degranulation Assay

One million neutrophils were incubated with 10 μM CB for 5 min at 37° C. prior to stimulation with fMLP (500 nM) for another 10 min. The reaction was stopped by being placed on ice, and the suspension was centrifuged at 500 g for 5 min at 4° C. Supernatants were assayed for MPO and MMP contents using the EnzChek Myeloperoxidase Activity Assay Kit and EnzChek Gelatinase/Collagenase Assay kit (Life Technologies, Grand Island, N.Y.), respectively.

### LPS-Induced Lung Injury

Mice were anesthetized with ketamine/Xylazine (100 mg/kg and 10 mg/kg). A 22G catheter (Jelco, Smiths Medical) was guided 1.5 cm below the vocal cords, and LPS (50 μl, 1 mg/ml, E. coli 011:B4) was instilled, while mice postures were maintained upright. Twenty-two hours after the induction of injury, 100 μl of FITC-labeled albumin (10 mg/ml) was injected via retro-orbital vein, and 24 hours after the induction of injury, mice were euthanized for sample collection. To obtain bronchoalveolar lavage fluid, 1 ml of PBS was instilled into lungs and retrieved via a tracheal catheter. For the baseline permeability measurement, saline without LPS was administered the same way. The baseline permeability measurement was subtracted in the data presented.

In survival experiments, mice were first administered retro-orbitally with 100 μl of alpha-GalCer at 10 μg/ml. Twelve hours later, mice were administered orotracheally with LPS (50 μl, 30 mg/ml, E. coli 055:B5).

### Acid Aspiration-Induced Lung Injury

Mice were anaesthetized by ketamine/Xylazine (1 gm/kg and 100 mg/kg) and were secured vertically from their incisors on a custom-made mount for orotracheal instillation. A 22G catheter (Jelco, Smiths Medical) was guided 1.5 cm below the vocal cords, and 2.5 μl/g of 0.05 M HCl was instilled. Four hours after the induction of injury, 100 μl of FITC-labeled albumin (10 mg/ml) was injected via retro-orbital vein. Mice were euthanized for sample collection 6 hours after the induction of injury. For the baseline permeability measurement, saline without HCl was administered the same way. The baseline permeability measurement was subtracted in the data presented.

In survival experiments, mice received 2.5 μl/g of 0.1 M HCl orotracheally and the observation period was extended up to 30 h.

### Quantification of Lung Histological Sections

Acute lung injury indices were quantified using HE-stained lung sections. The quantification of perivascular interstitial edema was done as the ratios of perivascular interstitial edema areas to vessel areas. More than 8 sections from the same lobes of the lungs were quantified for each mouse.

### Measurement of ROS in Neutrophils of Injured Lungs

Fifteen min after HCl ALI-induction, BALs were collected from the lungs. Mouse lungs were then mechanically dissociated and filtered through a 40 μm mesh to generate a single-cell suspension, and red blood cells were lysed. BAL cells, which were pelleted and resuspended, and whole lung cells were labeled for 30 minutes at 37° C. with 1 μM CM-H2-DCFDA (C6827, Invitrogen). The cells were then labeled for the surface markers (CD45; BD Bioscience 564279; Ly-6G; BD Bioscience 560602). Flow cytometry was performed on a BD LSRII.

### GST Pulldown Assay

Recombinant proteins were expressed in E coli and purified by affinity chromatography. The proteins were then incubated in 200 μl of the binding buffer (10 mM HEPES pH 7.4, 150 mM NaCl, 1% Triton, 0.12% SDS, 1 mM dithiothreitol, 10% glycerol, 1×protease inhibitor cocktail) at 4° C. overnight on a shaker. Next morning, glutathione beads were added to the protein mixture for additional 2 h. After extensive washes, proteins on the beads were resolved by SDS/PAGE and detected by Western Blot.

### In Vitro Kinase Assay

In 50 μl reaction buffer (100 mM Tris-HCl pH 7.4, 50 mM EGTA, 100 mM MgCl₂), recombinant MAP3K3 and/or MAP3K2 protein purchased from ThermoFisher Scientific was incubated with immune-precipitated substrate proteins or recombinant His-tagged p47^{phox} in the presence of cold ATP (50 μM) and/or [γ-³³P]-ATP (10 μCi) at 37° C. for 30 minutes. The reaction was stopped by adding the SDS loading buffer. The samples were boiled for 5 minutes. The proteins were separated by SDS-PAGE, and were visualized and quantified by a phosphoimager or analyzed by Western blotting.

### Human Neutrophils

Buffy coat of human blood samples were subjected to neutrophil enrichment using the EasySep Human Neutrophil Enrichment Kit (Stemceli Technologies) according to manufacturer's protocol. Briefly, the depletion antibody cocktail was mixed with the buffy coat followed by incubation with magnetic particles. The EasySep Magnet was then used to immobilize unwanted cells as the label-free neutrophils were poured into another conical tube. Enriched neutrophils were pelleted and resuspended in an assay buffer (Hanks buffer with Ca²⁺ and Mg²⁺, 0.25% BSA) for ROS production assay or Western analysis.

Bi-Layer Co-Culture of Neutrophils with Endothelial Cells

Mouse primary lung endothelial cells (MLEC) were first plated on the outside of the polycarbonate membrane (25,000 cells/cm²) of the Transwell inserts (24-well type, 0.4-μm pore size, Corning, Inc. 353095), and placed upside down in the wells of the culture plate. After the cells had adhered, the Transwell inserts were inverted and reinserted into the wells of the plate. The medium was replaced 24 h after seeding with serum-free medium. SOD (60 U/ml), catalase (100 U/ml), or mock were added to the lower chambers 2 h later for 30 min. Mouse neutrophils stimulated with 5 μM fMLP were then plated on the top surface of the insert (6×10⁶ cells/cm²) for 30 min. At the end of the incubation period, neutrophils on the top side of the inserts were removed by cotton swabs, and endothelial cells on the other side of the inserts were lysed with SDS-PAGE sample buffer for Western analysis.

### Trans-Endothelial Electrical Resistance (TEER) Measurement

ECIS 8W10E+ arrays (Applied BioPhysics) were coated with 10 μg/ml of poly-D-lysine (PDL) and washed with sterile water. Complete EBM-2 media (300 μl) was added to each well for a quick impedance background check. Subsequently, immortalized mouse pulmonary endothelial cells were seeded in a density of 60,000 cells/well in 300 μl EBM-2 medium in the coated arrays and incubated them at 37° C. in a CO₂ incubator. Electrical resistance of the cell layer was recorded continuously on an ECIS system (Applied BioPhysics) until a stable resistance of approximately 600-700 ohms was achieved, after which media were removed from wells and replaced with 100 μl of assay buffer (Hanks buffer with Ca² and Mg²⁺0.25% BSA). Cells were allowed to re-equilibrate at 37° C. for 2 hours, before 1 μl of SOD (60 U/ml), catalase (100 U/ml), or mock were added to wells for 30 min followed by addition of 50 μl of mouse neutrophils in assay buffer containing 5 μM fMLP. Data were collected real-time throughout the experiment. All ECIS measurements were analyzed at an AC frequency of 4 kHz, which was identified as the most sensitive frequency for this cell type by frequency scans along an entire frequency range (1 kHz-64 kHz). The TEER values were normalized against those co-cultured with WT neutrophils treated with mock.

### Sample Preparation for Single Cell RNA Sequencing

Lungs were perfused with PBS to remove the blood and minced with scissors, followed by incubation with pre-warmed collagenase solution (2 mg/ml in PBS with Ca²⁺/Mg²⁺) for 1 hour at 37° C. with mild agitation. The resulting single cell suspension was filtered through a 40 μm nylon cell strainer, and erythrocytes were lysed using a lysing buffer. Cells were resuspended in cold 0.1% BSA/PBS. Following live/dead staining with viability dye (Fixable Viability Dye eFluor 506, eBioscience), cells were incubated with a Fc-blocking reagent (BD Biosciences) for 5 minutes at 4° C. and an anti-CD45.2 mAb-PE-Cy7 antibody for 1 hour at 4° C. Cells were then sorted using a 100 m nozzle and 40 psi pressure (FACSAria instrument, BD Biosciences).

### Single-Cell RNA-Seq

Single-cell 3′ RNA-seq libraries were prepared using Chromium Single Cell V3 Reagent Kit and Controller (10× Genomics). Libraries were assessed for quality and then sequenced on HiSeq 4000 instruments (Illumina). Initial data processing was performed using the Cell Ranger version 2.0 pipeline (10× Genomics). Loupe Browser files for mouse datasets were generated using aggregate function in Cell Ranger pipeline with normalization on mapped reads and can be viewed using Single Cell Browser (10× Genomics). Post-processing, including filtering by number of genes expressed per cell, was performed using the Seurat package V2.3.4 and R 3.5.3. Following clustering and visualization with t-Distributed Stochastic Neighbor Embedding (t-SNE). Identification of cell clusters was performed on the final aligned object guided by marker genes. Differential gene expression analysis was performed for each cluster between cells from WT and KI mice. t-SNE plots and violin plots were generated using Seurat. Gene expression data were analyzed for enrichment using GSEA software (Broad Institute-version 3.0) and MSigDB version 6.2. The RNA sequencing data are deposited at Gene Expression Omnibus (GEO; access number: GSE134365 with Token “ypglyscoxhizron”).

### Patients, Intervention, and Data Collection

A pilot clinical study for validation of the therapeutic potential of pazopanib was performed with 5 pairs of lung transplantation patients who underwent single LT (each of paired recipients received one lung from the same donor). These represent all of the patients who were eligible for single LT and consented for enrollment into the study between Mar. 1, 2018 and Aug. 31, 2018. The paired patients were randomized to receive pazopanib 200 mg before surgery and no intervention, respectively. The baseline characteristics, surgical information, medical records during their ICU stay, as well as ventilator parameters, arterial blood gas analysis, and chest X-ray results within 5 days after LT were collected. All of the five donors were enrolled in a voluntary organ donation program and died of accident or disease.

The chest X-ray scores were obtained in the following manner: the heart field was regarded as the center, and the lung field was divided into four quadrants. Non-opacified regions, 0 points; opacified regions limited to 1/4 lung area, 1 point; limited to 2/4 lung area, 2 points; limited to 3/4 lung area, 3 points; in all lung fields, 4 points. Scores were independently collected by a clinician and a radiologist who were blinded for the treatment, and the average was used. If the difference between the two evaluators was greater than 1 point, discussion was conducted to reach a consensus. Hypoxemia index=PaO₂/FiO₂, measured by arterial blood gas analysis.

### Statistical Analysis and Study Design

For mouse studies, minimal group sizes for studies were determined by using power calculations with the DSS Researcher's Toolkit with an α of 0.05 and power of 0.8. Animals were grouped unblinded, but randomized, and investigators were blinded for most of the qualification experiments. No samples or animals were excluded from analysis. Assumptions concerning the data including normal distribution and similar variation between experimental groups were examined for appropriateness before statistical tests were conducted. Comparisons of means between two groups were performed by unpaired, two tailed t-test. Comparisons between more than two groups were performed by one-way ANOVA, whereas comparisons with two or more independent variable factors by two-way ANOVA using Prism 8.0 software (GraphPad). For Kaplan Meier survival analysis, logrank test was used. Statistical tests were using biological replicates. P<0.05 is considered as being statistically significant.

In the pilot clinical study, the linear mixed model repeated measures analysis was performed to compare the hypoxemia and X-ray scores over time between two groups accounting for both within-subject correlation and correlation of paired recipients. Time by group interaction was included in the model to examine the difference in trajectory of outcomes between two groups. Linear contrast was used to compare difference at each post-transplant day, and overall mean difference between groups as well. The analysis was conducted with SAS version 9.4 (SAS Inc., Cary, N.C.). The significance was set as p<0.05, two-sided.

### Results:

MAP3K2 and MAP3K3 Inhibit ROS Production from Neutrophils

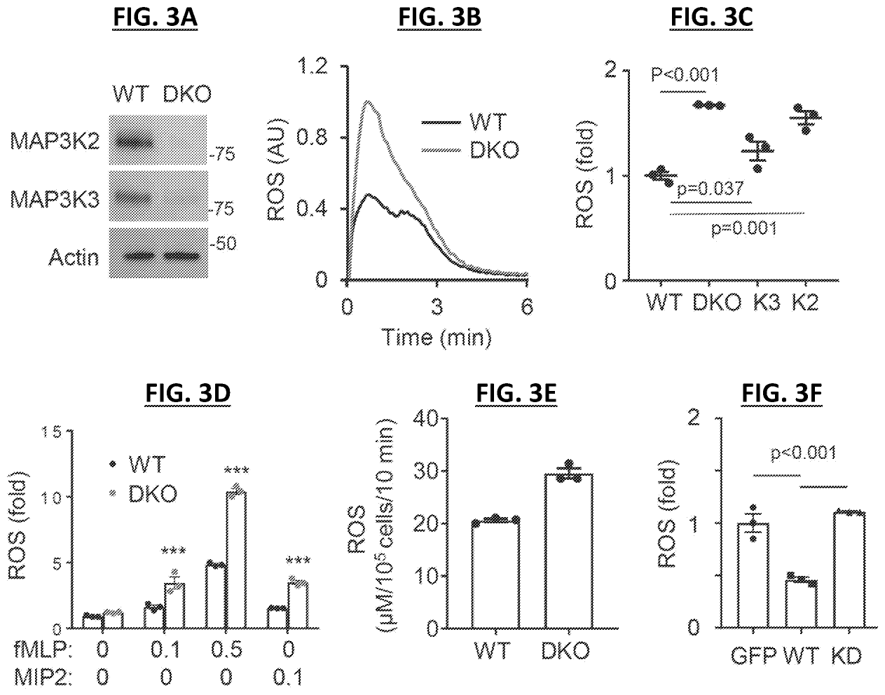

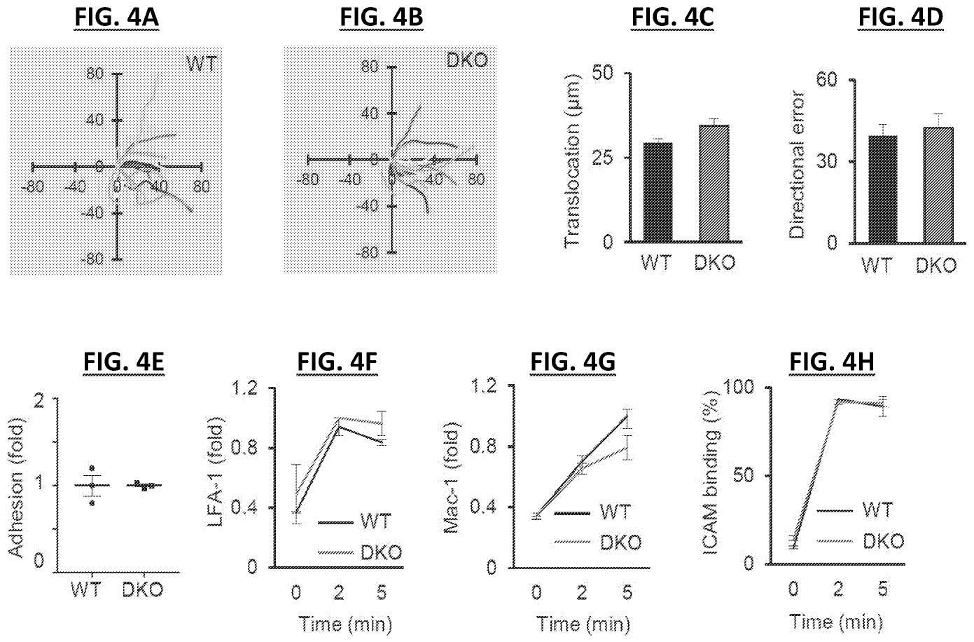

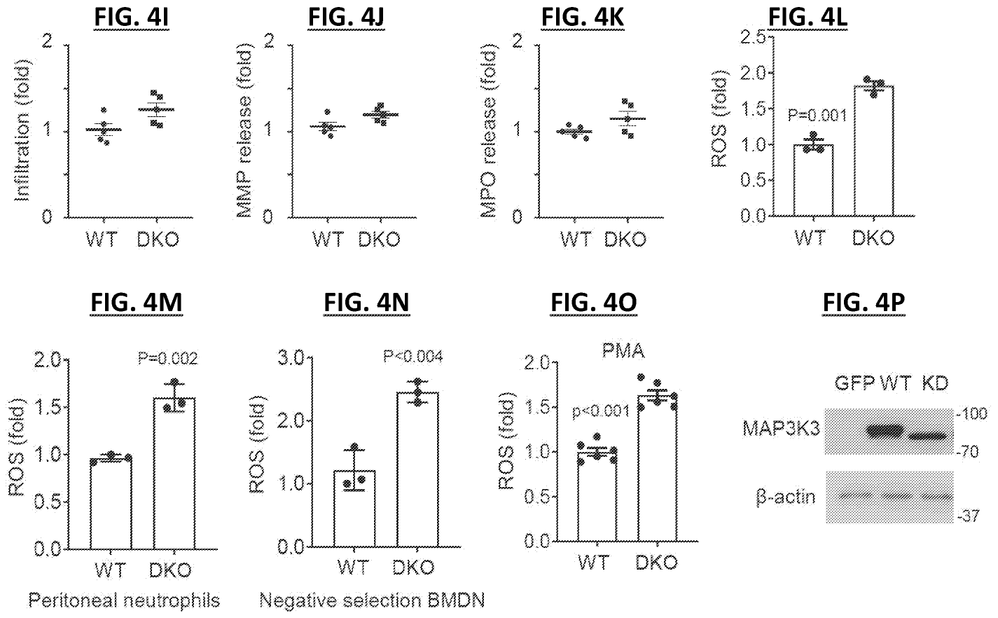

In mice, the Map3k3 gene is expressed abundantly in various hematopoietic cells with its expression being highest in myeloid cells. In addition, its close homolog Map3k2 is also expressed in mouse myeloid cells. Both MAP3K2 and MAP3K3 proteins could be readily detected in neutrophils by Western analysis (FIG. 3A). To understand the role of this MEKK subfamily in regulation of neutrophil functions, a battery of function tests were performed using MAP3K2/3-deficient neutrophils isolated from Map3k2^{-/-}Map3k3^{f/f}LyzCre mice. MAP3K2/3-deficiency did not affect neutrophil chemotaxis in vitro (FIGS. 4A-4D), neutrophil adhesion to endothelial cells under shear flow (FIG. 4E), or expression or activation of β2 integrins (FIGS. 4F-4H). Concordantly, the deficiency did not significantly affect neutrophil infiltration into inflamed peritonea in an in vivo neutrophil recruitment model (FIG. 4I). In addition, MAP3K2/3-deficiency did not significantly alter neutrophil degranulation (FIGS. 4J and 4K). However, the MAP3K2/3-deficiency led to increased total (measured by luminol) or released (measured by isoluminol or cytochrome C) ROS from neutrophils upon stimulation by fMLP (FIGS. 3B-3E, FIGS. 4L-4N), MIP2 (FIG. 3D), or PMA (FIG. 4O). While individual MAP3K knockouts showed significant elevations in ROS production, their effects appeared to be less than the double knockout (FIG. 3C), consistent with the idea that these two kinases are functional redundant. Expression of wild type (WT), but not kinase-dead (KD), MAP3K3 in the MAP3K2/3-deficient neutrophils could suppress the ROS release, indicating the importance of the kinase activity in regulation of ROS release (FIG. 3E & FIG. 4P).

MAP3K2/3-Deficiency Protects Mice from ALI

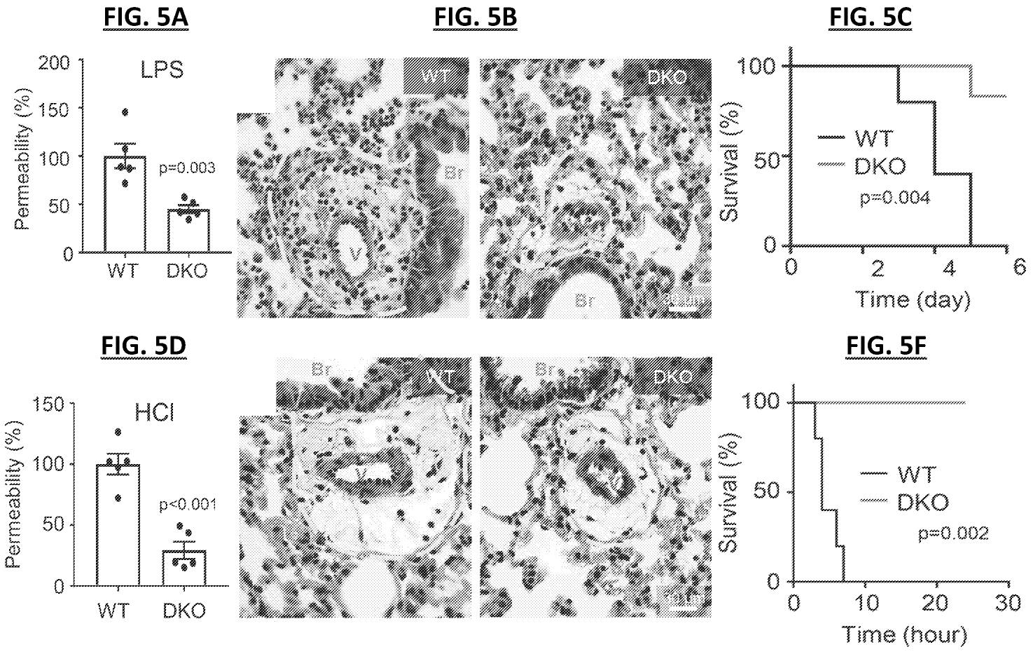

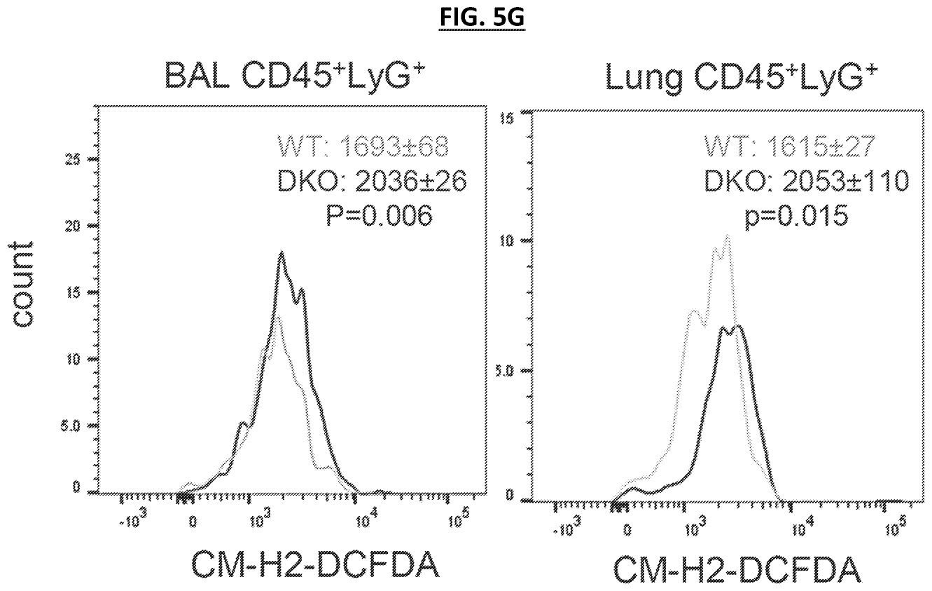

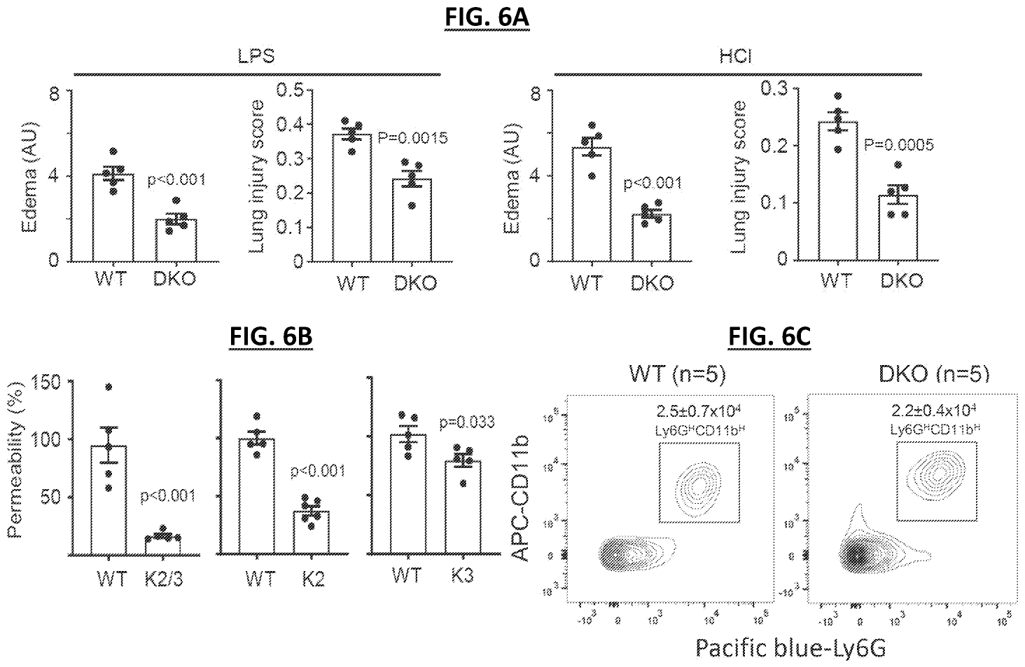

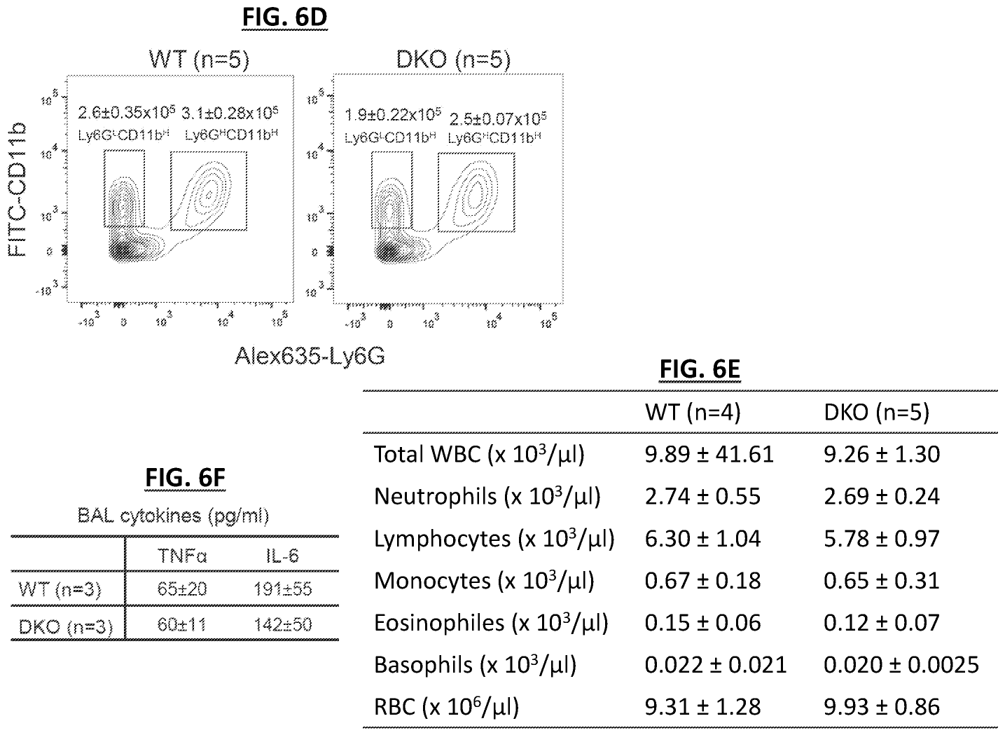

Given importance of neutrophils in ALI, the effects of the lack of these two MAP3Ks were assessed in mouse ALI models. To limit contributions from non-hematopoietic cells, an adoptive bone marrow transfer was performed from the Map3k2^{-/-}Map3k3^{f/f}LyzCre mouse line, to lethally irradiated WT recipient mice. The resultant mice are designated as DKO, which lacks MAP3K2 in all hematopoietic cells and MAP3K3 in myeloid cells. The DKO and their control mice that received bone marrow transfer from WT littermates were first subjected to LPS-induced ALI via orotracheal instillation of LPS. This ALI model recapitulates post-infection inflammation-induced lung injury with many hallmarks of human ALI including neutrophilic influx into the alveolar space, pulmonary edema, and increased lung permeability accompanied with high mortality. DKO mice had significantly lower pulmonary permeability and perivascular interstitial edema than the control mice (FIGS. 5A and 5B, FIG. 6A). The DKO mice also showed significantly reduced mortality compared to the WT control mice (FIG. 5C).

A different ALI model, which is induced by orotracheal instillation of HCl, was then tested. The HCl model recapitulates acid aspiration-induced ALI/ARDS in humans. This condition, also known as aspiration pneumonitis, results from pulmonary aspiration of the acid content of the stomach. This frequently occurs to patients with disturbed consciousness (e.g., drug overdose, seizures, cerebrovascular accident, sedation, anesthetic procedures) and in the frail older adults, as well as accounting for up to 30% of all deaths associated with general anesthesia. In the acid-induced ALI model, the DKO mice were also observed to have significantly lower pulmonary permeability and perivascular interstitial edema than the control mice (FIGS. 5D and 5E, FIG. 6A), as well as significantly reduced mortality compared to the WT control mice (FIG. 5F). Because acid-induced ALI is the result of direct insult of lung barrier cells without an involvement of complicated inflammatory reactions in LPS-induced ALI, the acid ALI model was used for further mechanistic investigations.

Myeloid-specific MAP3K2 KO (Map3k2^{f/f}LyzCre) and MAP3K2/3 DKO (Map3k2^{f/f}Map3k3^{f/f}LyzCre) mice were generated. Consistent with the ROS production from isolated neutrophils (FIG. 3C), myeloid-specific DKO appeared to have a greater effect on permeability than each individual myeloid-specific KO in the HCl ALI model (FIG. 6B). Myeloid cell numbers were examined and no significant difference in the numbers of myeloid cells in the injured lungs, bronchoalveolar lavage fluids, or circulation were observed between the DKO and WT control mice (FIGS. 6C-6E). In addition, there were no significant differences in the contents of TNFα or IL-6 in bronchoalveolar lavage fluids (FIG. 6F). These results together suggest that the lack of MAP3K2 and 3 in myeloid cells primarily impacts pulmonary permeability rather than myeloid infiltration or cytokine production in injured lungs.

Given that ROS is generally considered detrimental to tissue injuries, the beneficial effects of myeloid-specific MAP3K2 and/or 3-deficiency to acute lung injuries were not anticipated. To confirm that neutrophils lacking MAP3K2/3 indeed produce more ROS in injured lungs, the ROS of neutrophils in BAL and lungs subjected to HCl injury were measured by flowcytometry and elevated ROS production in MAP3K2/3-deficient neutrophils was observed compared to WT neutrophils in the injured lungs (FIG. 5G).

MAP3K2/3 Phosphorylates p47^{phox} at Ser208

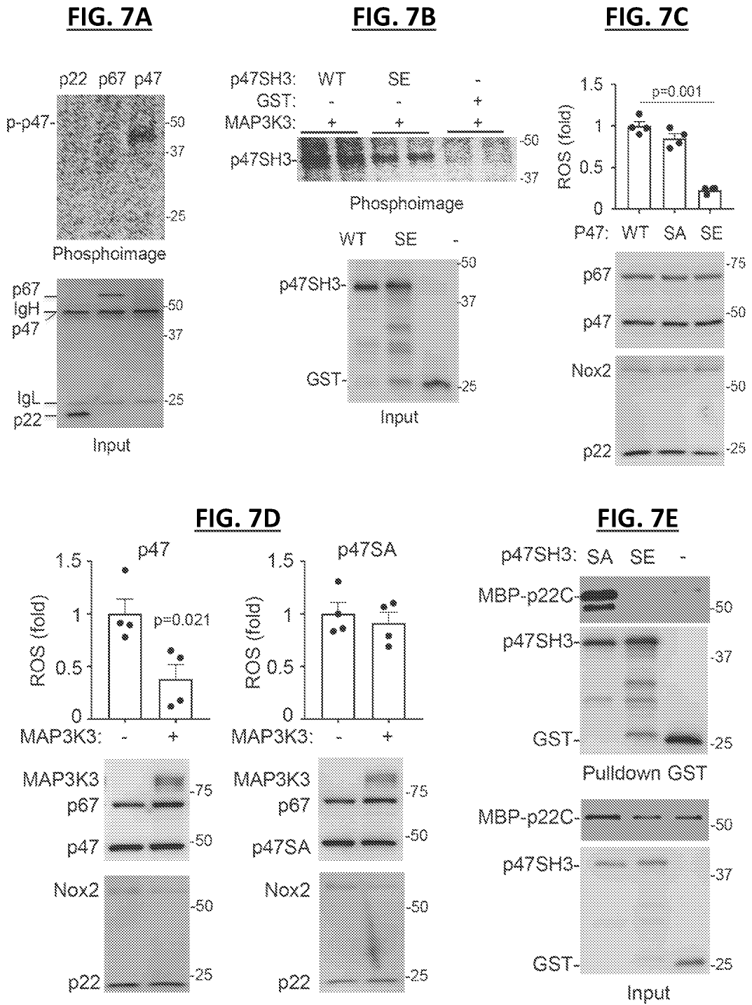

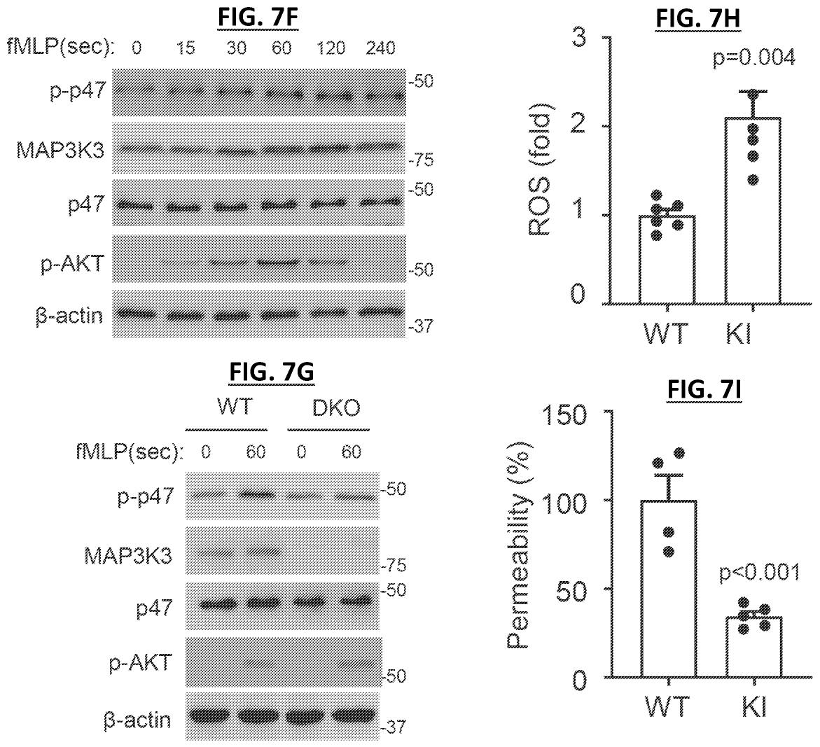

How MAP3K2 and 3 regulate ROS production in neutrophils was next investigated. Because the Nox2 complex is the major source of ROS released from neutrophils, the possibility of whether the kinases phosphorylate one of the subunits of the Nox2 complex was studied. An in vitro kinase assay was performed and it was found that p47^{phox}, but not p22^{phox}, p67^{phox} (FIG. 7A), gp91^{phox}, or p40^{phox} (data not shown) could be phosphorylated by MAP3K3. Though the phosphorylation site consensus sequence for MAP3K3 is unknown, the p47^{phox} sequence was analyzed using the Scansite run with reported peptide array data for a related kinase MAP3K5 to identify likely sites of phosphorylation. This analysis predicted Ser-208 of p47^{phox} as the best scoring site among those previously observed. When this site was mutated in a fragment (FIG. 7B) of p47^{phox}, MAP3K3-mediated phosphorylation was significantly reduced, indicating that this residue may be phosphorylated by MAP3K3.

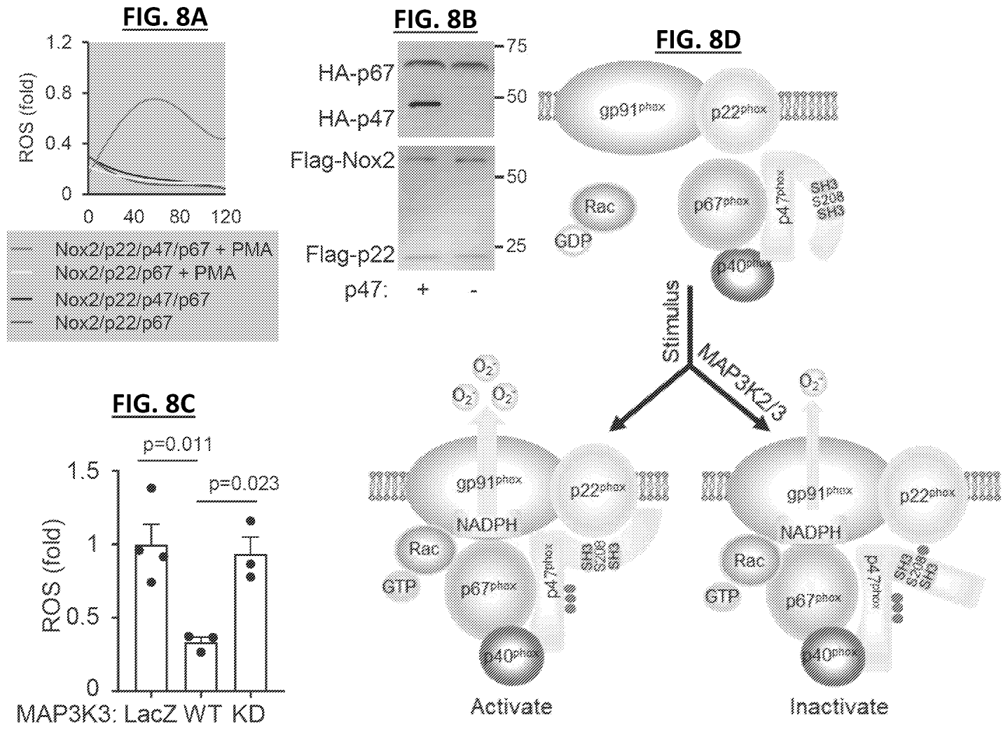

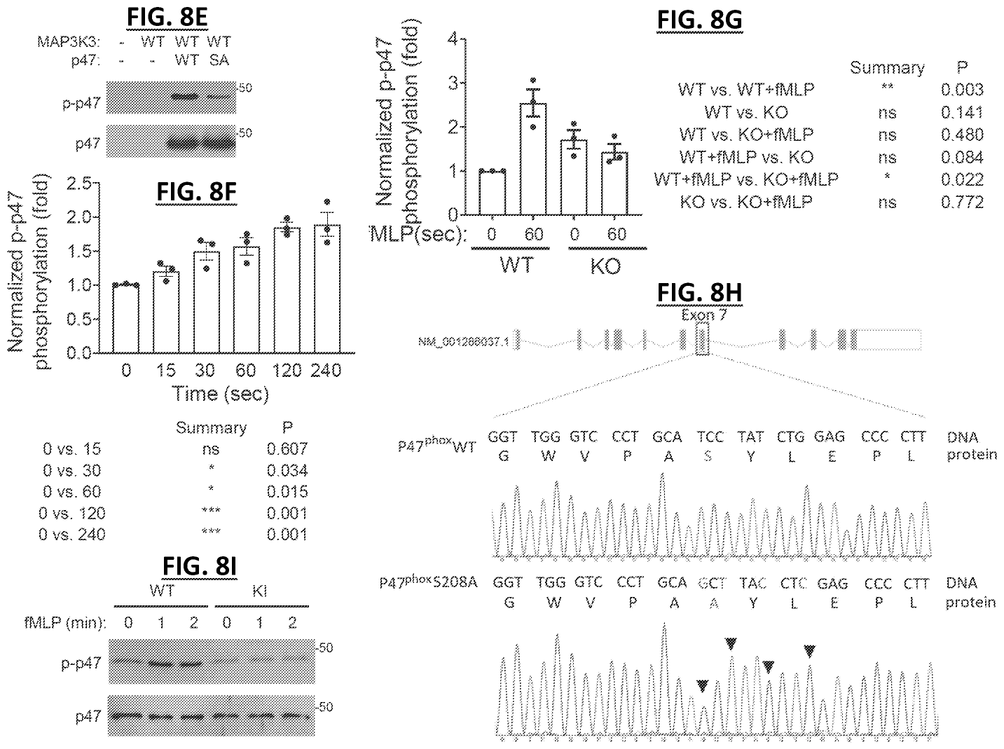

To determine the effect of this phosphorylation on the activity of the NDAPH oxidase, a reconstituted NADPH oxidase activity assay was run in COS-7 cells by expressing the NADPH oxidase subunits p47^{phox}, p67^{phox}, p40^{phox}, NOX2, and p22^{phox}. These proteins are either not or insufficiently expressed in COS-7 cells. Upon addition of PMA, production of ROS could be detected from the reconstituted COS-7 cells, and this ROS production is completely dependent on the exogenous expression of p47^{phox} (FIGS. 8A and 8B). Importantly, expression of WT MAP3K3, but not its kinase dead mutant, inhibited ROS production in this system (FIG. 8C). Thus, a ROS production system was developed that can be inhibited by MAP3K3, similar to what happens in neutrophils. When the phospho-mimetic p47^{phox} S208E mutant was used instead of WT in this reconstituted system, there was markedly reduced ROS production in comparison to WT p47^{phox} (FIG. 7C). The non-phosphorylatable S208A p47^{phox} mutant, by contrast, showed similar activity in this ROS reconstitution assay to the WT p47^{phox} (FIG. 7C). Moreover, expression of MAP3K3 was able to inhibit ROS production in cells expressing the WT p47^{phox}, but not those expressing non-phosphorylatable S208A p47^{phox} (FIG. 7D). These results together indicate that phosphorylation of p47^{phox} at S208 inhibits the NADPH oxidase activity. Because Ser-208 is located between two SH3 domains of p47^{phox}, which were involved in the interaction with p22^{phox} during activation of the NADPH oxidase complex (FIG. 8D), it was postulated that the phosphorylation at Ser-208 might interfere with this interaction, a critical step in NADPH oxidase activation. Indeed, the phosphomimetic Ser-208 to Glu mutation impeded the interaction of p47^{phox} with p22^{phox} in a pull-down assay (FIG. 7E).

To detect if p47^{phox} is phosphorylated in neutrophils by MAP3K2 and 3, a polyclonal antibody immunized with a p47^{phox} peptide containing phosphorylated Ser-208 was generated. The antibody showed preference for Ser-208-phosphorylated over non-phosphorylated p47^{phox}, because mutation of Ser-208 to alanine markedly diminishes the detection by the antibody in cells overexpressing MAP3K3 (FIG. 8E). Using the antibody, time-dependent increases in p47^{phox} phosphorylation at Ser-208 were detected (FIG. 7F, FIG. 8F). In addition, the MAP3K3 band upshift was observed in electrophoresis (FIG. 7F), which reflects its activation by fMLP. MAP3K3 activates via autophosphorylation. Importantly, this fMLP-induced increase in p47^{phox} phosphorylation detected by this antibody was not observed in neutrophils lacking MAP3K2/3 (FIG. 7G, FIG. 8G), suggesting that fMLP induces the phosphorylation of p47^{phox} at Ser-208 via MAP3K2 and 3. The bands detected in the DKO neutrophils by the anti-phospho-Ser-208 antibody may reflect weak detection of non-phosphorylated p47^{phox} by the antibody as shown in FIG. 8E. These data together strongly support the conclusion that MAP3K2 and 3 phosphorylate p47^{phox} S208 to regulate ROS production.

Knock-In Mutation of p47^{phox} Ameliorates ALI

To further assess the importance of p47^{phox} Ser-208 phosphorylation in ROS production and ALI, a knock-in (KI) mouse line was generated in which Ser-208 of p47^{phox} was replaced with alanine, designated as p47^{phox}-KI. DNA sequencing confirmed correct mutations introduced into the mouse line (FIG. 8H). In addition, Western analysis showed that fMLP failed to increase p47^{phox} S208 phosphorylation in neutrophils isolated from the p47^{phox}-KI mice in comparison to those from the WT mice (FIG. 8I). Consistent with the idea that p47^{phox} S208 phosphorylation suppresses ROS production, neutrophils from the p47^{phox}-KI mice produced significant greater amounts of ROS upon stimulation (FIG. 7H). Importantly, the mice receiving bone marrow transfer from the p47^{phox}-KI mice also showed reduced pulmonary permeability compared with mice receiving WT bone marrow transfer in HCl-induced lung injury (FIG. 7I). These data indicate that p47^{phox} S208 phosphorylation, which is dependent on MAP3K2/3 in neutrophils, suppresses ROS production, and the lack thereof reduces pulmonary permeability during ALI.

Paracrine H₂O₂ Enhances Endothelial Cell Barrier Function

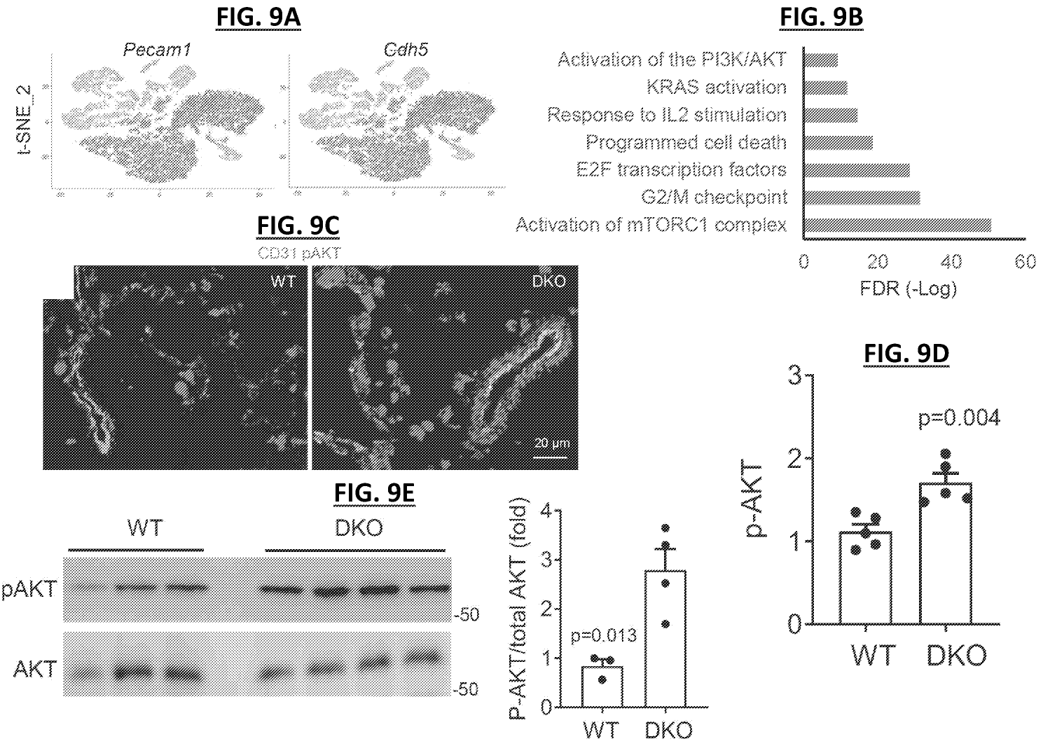

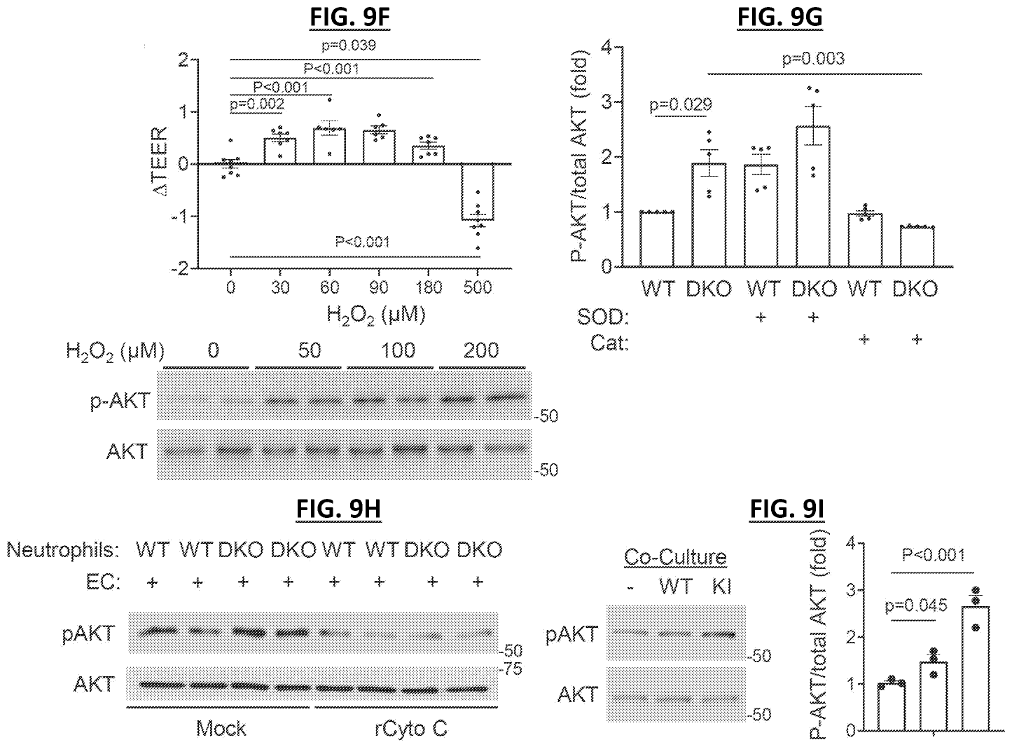

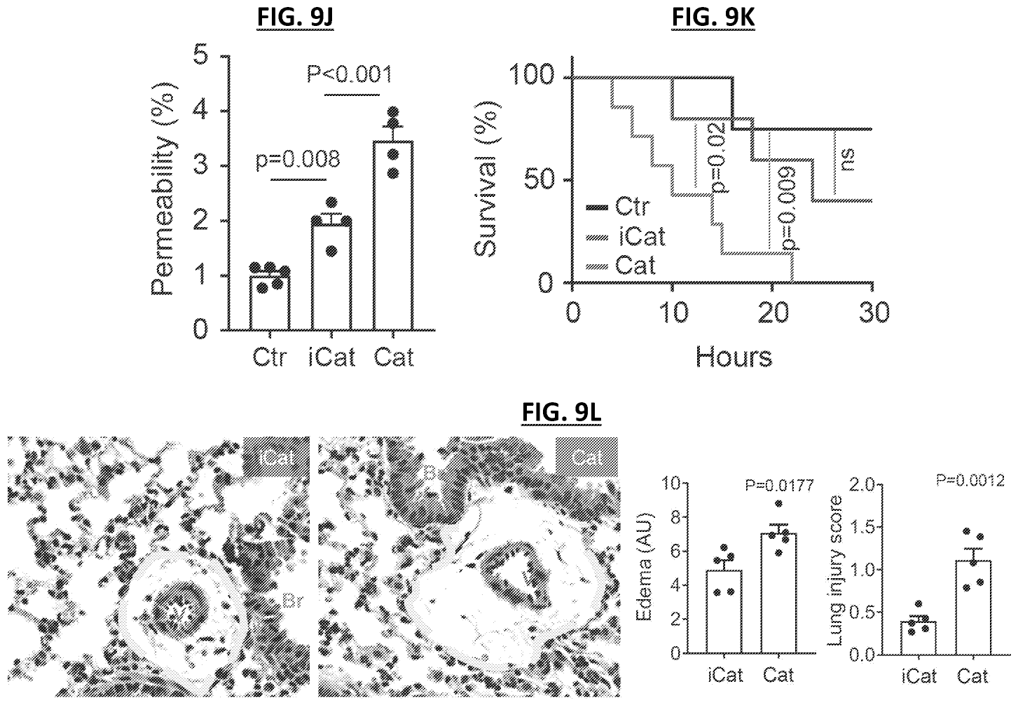

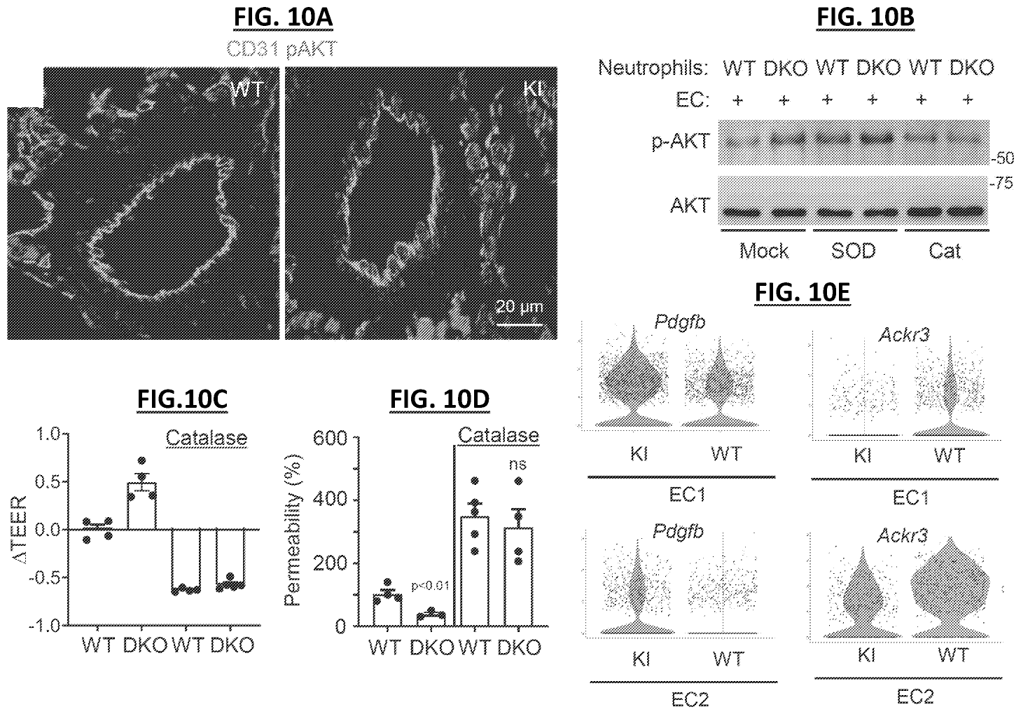

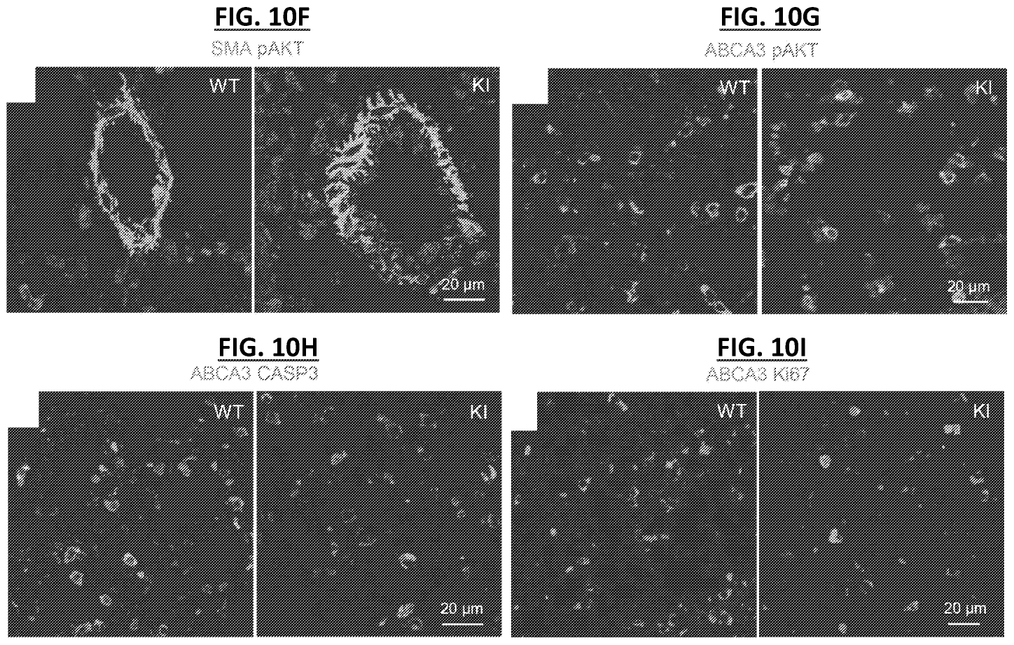

The aforementioned genetic data indicate that neutrophil ROS has to act on lung barrier cells to exert its anti-ALI effects. To understand the underlying mechanism, single cell RNA sequencing of CD45-negative cells sorted from WT and p47^{phox}-KI lungs subjected to HCl-induced ALI was performed. Endothelial cells were identified by high expression of Pecam1 and Cdh5 (FIG. 9A). Pathway enrichment analysis revealed that some of the signaling pathways altered by p47^{phox} KI were related to AKT signaling (FIG. 9B). Consistent with the scRNAseq data, immunofluorescence staining of injured lung sections from DKO (FIG. 9C), or p47^{phox}-KI mice (FIG. 10A, FIG. 9D) showed increased levels of AKT phosphorylation in pulmonary endothelial cells marked by CD31 compared to their corresponding WT controls. There was also elevated AKT phosphorylation in the DKO lung extracts compared to the controls (FIG. 9E).

ROS, once being released from myeloid cells, is quickly converted into H₂O₂ in lungs. H₂O₂ stimulates AKT phosphorylation in endothelial cells, and ATK activation in endothelial cells strengthens vessel barrier integrity and has a protective role in a murine model of ALI by preventing capillary leakage and clearing alveolar fluid. H₂O₂ at low concentrations enhanced trans-endothelial electrical resistance (TEER) of primary cultured mouse lung endothelial cells and stimulates AKT phosphorylation in these cells (FIG. 9F). To determine if H₂O₂ mediates the action of hematopoietic loss of MAP3K2/3 or p47^{phox} phosphorylation on endothelial cells, a co-culture of WT and mutant neutrophils with mouse primary lung endothelial cells was performed. Mouse primary lung endothelial cells co-cultured with activated MAP3K2/3 DKO neutrophils had elevated AKT phosphorylation compared to co-culture with WT neutrophils (FIG. 10B, FIG. 9G). This phospho-AKT elevation could be abrogated by the presence of catalase or reduced cytochrome C, but not superoxidase dismutase (SOD) (FIG. 10B, FIGS. 9G and 9H). Catalase and reduced cytochrome C promote conversion of H₂O₂ to water, whereas SOD converts superoxide to H₂O₂. Furthermore, co-culture of activated DKO neutrophils with mouse endothelial cells increased TEER over that of activated WT neutrophils, and this difference in TEER could also be abrogated by the presence of catalase (FIG. 10C). The increase in AKT phosphorylation were also observed with co-culture of p47^{phox}-KI neutrophils with mouse primary lung endothelial cells (FIG. 10I). Thus, these results collectively support the conclusion that MAP3K2/3-deficiency or p47^{phox}-KI causes sufficient increases in H₂O₂ to increase AKT activation in pulmonary endothelial cells, leading to enhancement of endothelial junction integrity. In addition, the catalase and SOD treatment results indicate that extracellular H₂O₂, rather than free superoxide radicals, plays a direct role in AKT activation and endothelial barrier function enhancement. This conclusion is further confirmed by intravenous administration of pegylated catalase to MAP3K2/3 DKO and corresponding WT control mice in the HCl-induced ALI model. Pegylated catalase treatment increased lung permeability and interstitial edema and decreased survival (FIG. 10D, FIGS. 9J-9L), confirming the importance of extracellular H₂O₂ in ALI protection (FIG. 10D). More importantly, pegylated catalase treatment abrogated permeability effect of MAP3K2/3-deficiency, indicating the importance of extracellular H₂O₂ in HCl-induced ALI protection rendered by MAP3K2/3-deficiency (FIG. 10D).

### P47phox-KI Remodels Pulmonary Barrier Cell Microenvironments

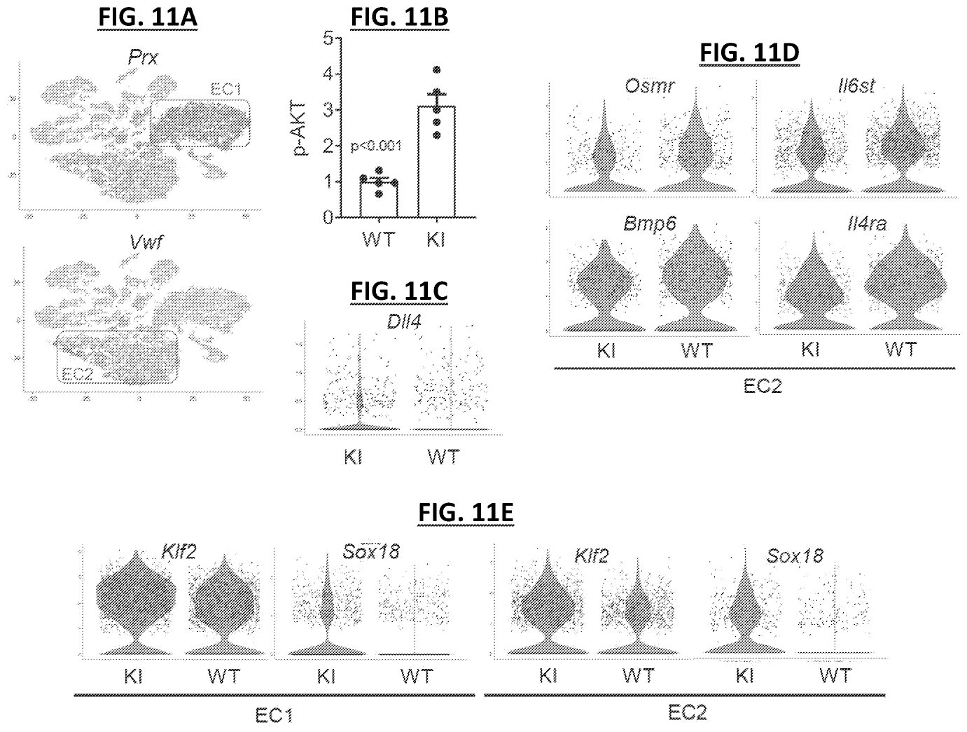

Further analysis of the scRNAseq data was performed by subdividing endothelial cells into EC1 for high Prx expression and EC2 for high Vwf expression (FIG. 11A). The EC1 cells are likely from capillary, whereas EC2 cells are probably derived from larger blood vessels. Among the differential expressed genes (Table 1), Pdgfb was found to be upregulated in both EC groups of the p47^{phox}-KI samples in comparison to WT ones (FIG. 10E). Endothelial PDGF acts on pericytes to enhance blood vessel integrity. As PDGF can stimulate AKT, increased AKT phosphorylation was observed in pericytes surrounding blood vessels in p47^{phox}-KI lung sections over WT ones (FIG. 10F, FIG. 11B). This EC2 upregulation of pdgfb expression, together with EC2 upregulation of Dll4 (FIG. 11C), which encodes the Notch ligand DLL4 and promotes pericyte survival and adhesion to endothelial cells, may contribute to reduction in interstitial edema shown above.

There were a number of downregulated signaling ligands or receptors in p47^{phox}-KI endothelial cells: Ackr3 (downregulated in both EC1 and EC2, FIG. 10E), Il6st, Osmr, Il4ra, and Bmp6 (downregulated in EC2) (FIG. 11D). Ackr3 encodes for CXCR7, a receptor for CXCL12, and its signaling disrupts endothelial barrier function. BMP6, IL6, which signals through IL6 receptor beta (Il6st) and oncostatin receptor (Osmr), and IL4, which signals through IL4 receptor alpha (Il4ra), induce endothelial hyperpermeability. Thus, the moderate elevation of ROS as the result of hematopoietic loss of p47^{phox} phosphorylation altered the pulmonary vasculature microenvironment by modulating expression of signaling ligands and receptors. Altered signaling from these ligands and receptors in turn result in further alterations in expression of genes, many of which are pertinent to enhancement of pulmonary vasculature integrity (Table 1). Notably among them are transcript factors Klf2 and Sox18 (FIG. 11E) that are known to be key players in vasculature barrier functions.

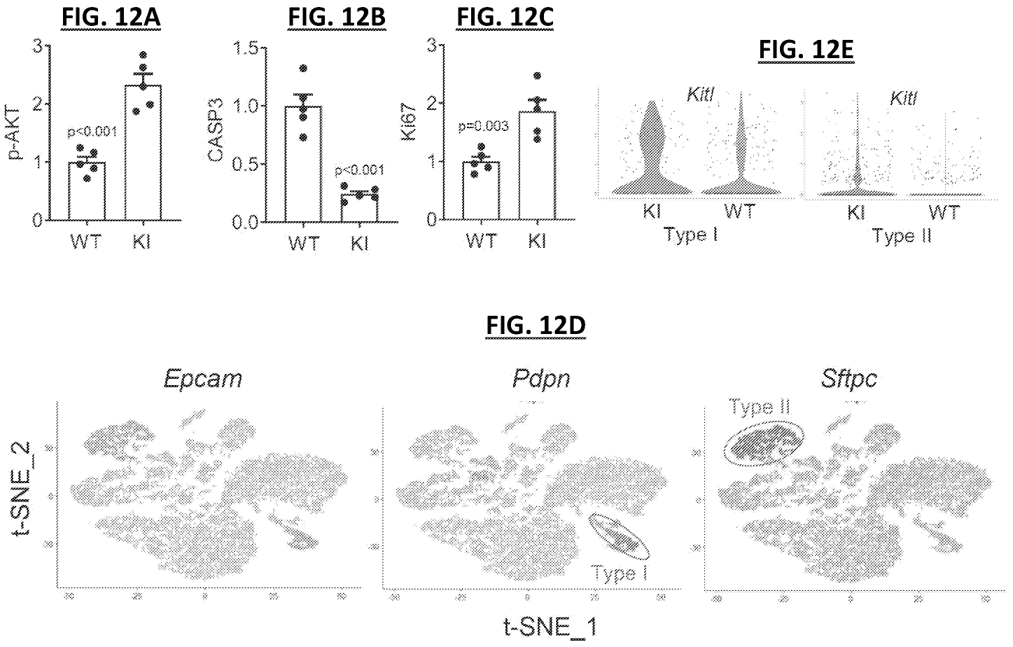

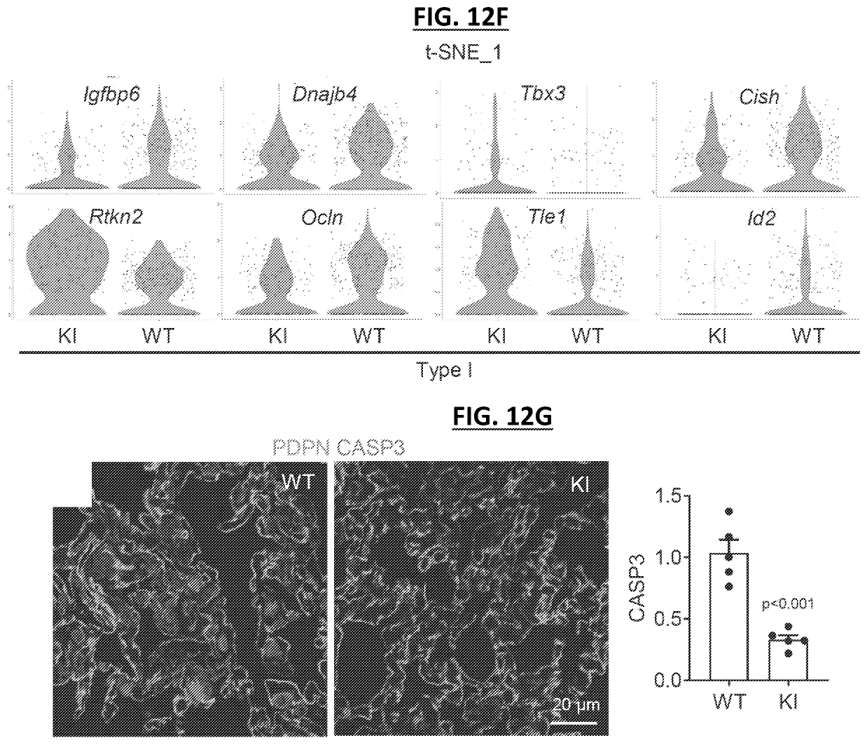

PDGF signaling is also important for lung alveolar formation and stimulates type II cell proliferation. The immunostaining of ALI lung sections also revealed increased levels of AKT phosphorylation in Type II epithelial cells marked by ABCA3 (FIG. 10G, FIG. 12A) from p47^{phox}-KI lungs compared to those of control lungs. Consistent with the roles of AKT signaling, reduced levels of the apoptosis marker activated caspase 3 and increased proliferation marker Ki67 were detected in the p47^{phox}-KI lung epithelial cells (FIGS. 10H and 10I, FIGS. 12B and 12C).

Two sub-populations of Epcam-high epithelial cells were identified by single cell RNA sequencing; one expressed high Pdpn, a marker for the type I alveolar cells, and the other expressed high Sftpc, a marker for the type II cells (FIG. 12D). Among the significantly differentially expressed genes (Table 1), Kit1 is a signaling ligand gene that was upregulated in both p47^{phox}-KI epithelial groups (FIG. 12E). Kit1 encodes SCF and has important roles in alveolar maintenance and lung epithelial cell proliferation. A group of genes differentially expressed in the type I group between p47^{phox}-KI and WT was also noted, whose changes skewed towards anti-apoptosis (FIG. 12F). In a non-limiting aspect, these changes help to explain reduced activated caspase 3 staining in these type I cells from the p47phox-KI ALI lungs (FIG. 12G), while lacking notable increases in AKT phosphorylation. All these data together indicate that moderate elevation of ROS from neutrophils exerts very board effects on pulmonary barrier cells.

TABLE 1
Differential expressed genes in endothelial cells

| gene | p_val | avg_logFC |
|---|---|---|
| Mpz | 0.521635296 | 0.635573145 |
| Hbb-bs | 2.28171E−14 | 0.623243784 |
| Ramp2 | 0.00189341 | 0.547595593 |
| Spp1 | 0.000641322 | 0.513586767 |
| Mgp | 0.005486122 | 0.464725072 |
| Hpgd | 0.000205882 | 0.459362768 |
| Ly6a | 0.209748751 | 0.455889196 |
| Ctla2a | 0.331903621 | 0.428246203 |
| Selenbp1 | 1.68551E−18 | 0.415868361 |
| Tinagl1 | 2.99514E−10 | 0.40584745 |
| Retnla | 0.71992073 | 0.405078275 |
| Chd9 | 1.07856E−21 | 0.393543599 |
| Dcn | 3.22571E−05 | 0.380579896 |
| Ace | 0.081592381 | 0.371855902 |
| Pmp22 | 0.172388283 | 0.369288903 |
| Gm26870 | 0.09897758 | 0.367349888 |
| B2m | 3.19902E−07 | 0.365208389 |
| Slc9a3r2 | 3.37539E−06 | 0.33898112 |
| Cdh5 | 0.401653054 | 0.334002944 |
| Lyve1 | 0.162799319 | 0.324269902 |
| Tmem100 | 0.183044681 | 0.322975603 |
| Txnip | 1.30257E−10 | 0.308760667 |
| Igfbp7 | 0.082285166 | 0.305817482 |
| Tsc22d3 | 5.70077E−10 | 0.303828666 |
| Cav1 | 0.042805574 | 0.303419416 |
| Ccdc141 | 3.9933E−13 | 0.283166716 |
| Tspan13 | 0.961029664 | 0.280971925 |
| Kitl | 0.003303725 | 0.277335368 |
| Arl6ip1 | 6.93227E−07 | 0.275635293 |
| Calcrl | 0.318639034 | 0.273319484 |
| Fmo2 | 1.87433E−07 | 0.271140994 |
| Klf2 | 0.004868597 | 0.268656029 |
| Egfl7 | 0.865781815 | 0.262024393 |
| Plpp1 | 0.654246578 | 0.259558578 |
| Exosc7 | 1.66671E−09 | 0.258306281 |
| Rhob | 0.00181835 | 0.253301351 |
| AC149090.1 | 5.34039E−09 | 0.252676803 |
| Sema3c | 0.031355307 | 0.251336237 |
| Pmvk | 0.000518515 | −0.254280781 |
| Rhou | 1.58023E−15 | −0.256328116 |
| Ttc9 | 7.66423E−13 | −0.256958775 |
| Chchd10 | 5.92589E−14 | −0.264381941 |
| Aldh3b1 | 0.072997423 | −0.266587736 |
| Areg | 0.012503043 | −0.267671749 |
| Neat1 | 1.78565E−08 | −0.275629261 |
| Tuba1a | 0.305992966 | −0.277953043 |
| Scd1 | 8.16748E−09 | −0.283155037 |
| Gm47283 | 1.56004E−18 | −0.28436452 |
| Eif4h | 9.90211E−13 | −0.295455028 |
| Car8 | 5.72191E−14 | −0.296508174 |
| Nfe2l2 | 6.17657E−16 | −0.303643677 |
| Ezr | 4.76026E−13 | −0.306711578 |
| Cd24a | 0.661342282 | −0.311266504 |
| Hp | 8.27403E−13 | −0.319960421 |
| Srxn1 | 1.8804E−09 | −0.328600277 |
| Tppp3 | 0.019392716 | −0.32905181 |
| Cldn4 | 0.129794589 | −0.336838526 |
| Fabp5 | 8.87953E−06 | −0.340621937 |
| Lrg1 | 1.42246E−06 | −0.342698396 |
| Por | 2.81699E−25 | −0.361929945 |
| Krt8 | 4.36171E−11 | −0.367056612 |
| Chia1 | 1.55787E−08 | −0.368746504 |
| Cbr2 | 1.57242E−21 | −0.369341836 |
| Txnrd1 | 3.14368E−23 | −0.412492521 |
| Hist1h1c | 3.66177E−14 | −0.422535021 |
| Scgb1a1 | 1.46488E−07 | −0.429552822 |
| Osgin1 | 1.1147E−11 | −0.437193629 |
| Sfn | 2.96875E−08 | −0.474692493 |
| Gclc | 1.55245E−24 | −0.496796783 |

### Pazopanib is a Substrate Specific Inhibitor of MAP3K2/3

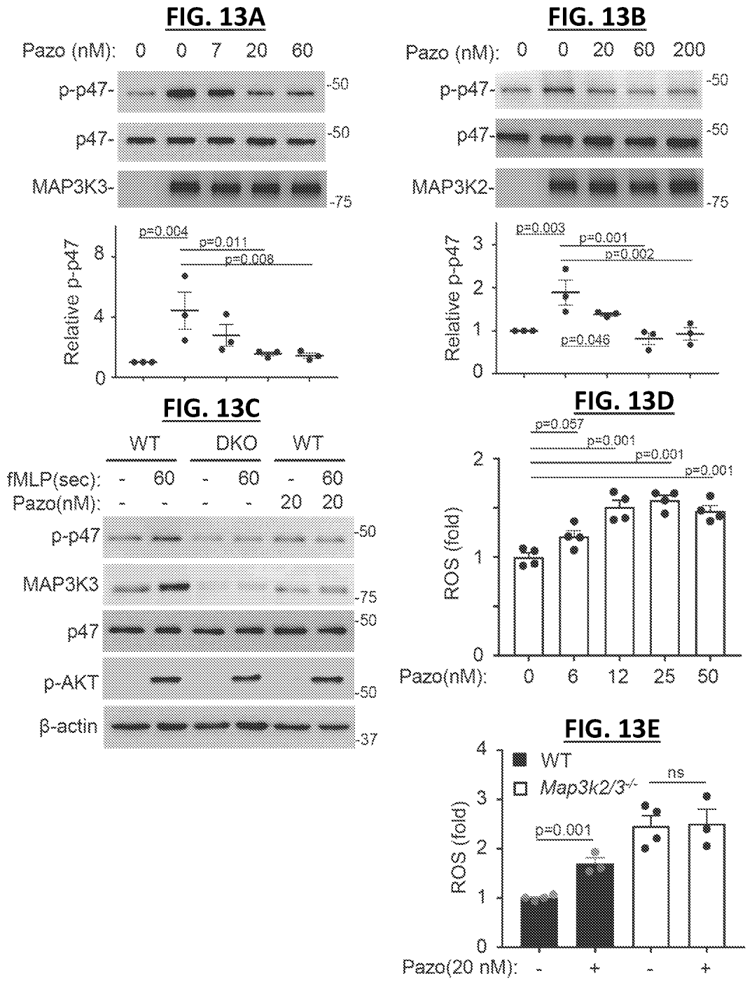

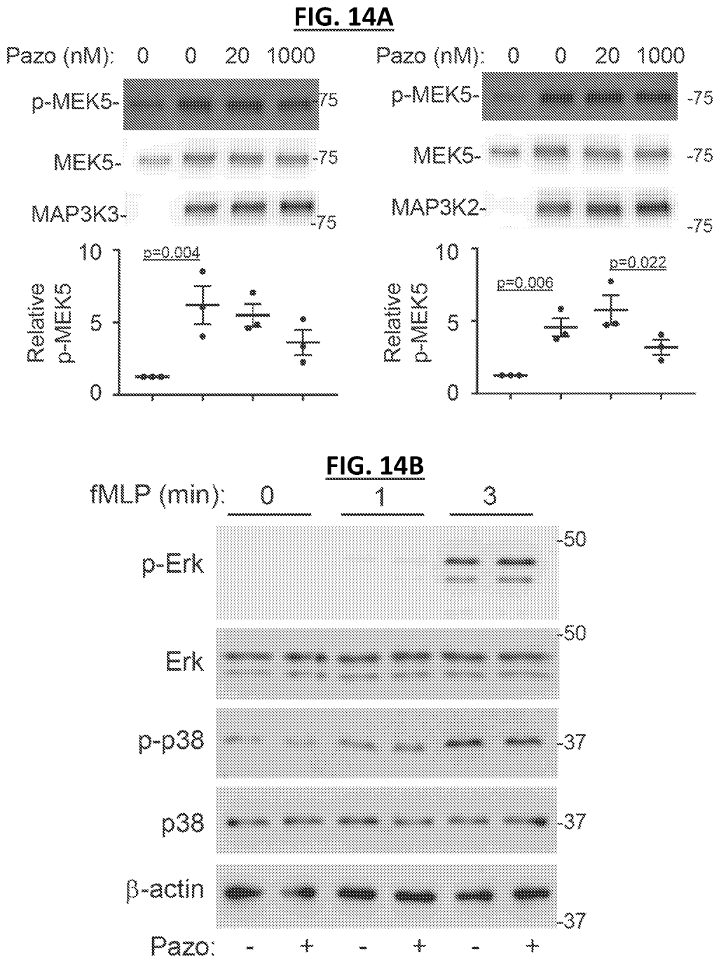

The aforementioned genetic results suggest that the subfamily of MAP3K2 and 3 kinases would be a potential therapeutic target for treating ALI. Previous screens for MAP3K2 inhibitors have identified a number of small molecule inhibitors with >1 μM IC₅₀ when assayed with MEK5 as a substrate (Ahmad, et al., 2013, J. Biomol. Screen. 18:388-399; Ahmad, et al., 2015, Biochem. Biophys. Res. Commun. 463:888-893). Six of these candidates, including Sunitinib, Pazopanib, Bosutinib, Ponatinib, Immatinib and Nintedanib, were tested in an in vitro kinase assay. Unexpectedly, pazopanib, but not others, inhibited p47^{phox} phosphorylation at Ser-208 by MAP3K2 and 3 at low nM IC₅₀ values (FIGS. 13A and 13B). Pazopanib is a VEGFR1 inhibitor and FDA-approved drug for targeted cancer therapy. Pazopanib inhibited MEK5 phosphorylation by MAP3K2 or 3 at >1 μM IC₅₀ values (FIG. 14A). Thus, pazopanib has an unprecedented substrate specificity, which would be a beneficial pharmacological feature as it would not inhibit MEK5 phosphorylation by MAP3K2/3 to cause un-intended effects mediated by MEK5.

Pazopanib was subsequently tested in mouse neutrophils and was found to inhibit p47^{phox} phosphorylation at Ser-208 (FIG. 13C). Pazopanib also abrogated the increase in MAP3K3 protein content induced by fMLP (FIG. 13C), suggesting it inhibits MAP3K3 activation in neutrophils. Importantly, treatment of WT (FIG. 13D), but not MAP3K2/3-deficient (FIG. 13E), mouse neutrophils with pazopanib led to increases in ROS production, indicating that pazopanib increases ROS production via MAP3K2 and 3 in neutrophils. In addition, pazopanib did not affect ERK or p38 phosphorylation in mouse neutrophils (FIG. 14B).

### Pazopanib Ameliorates ALI

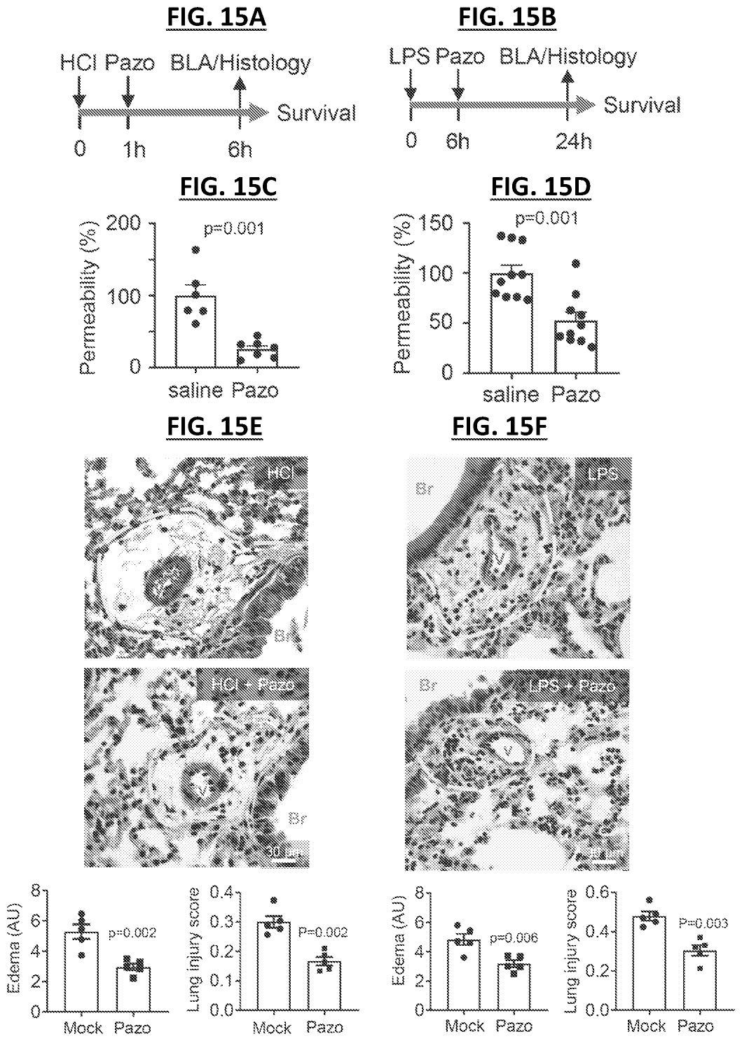

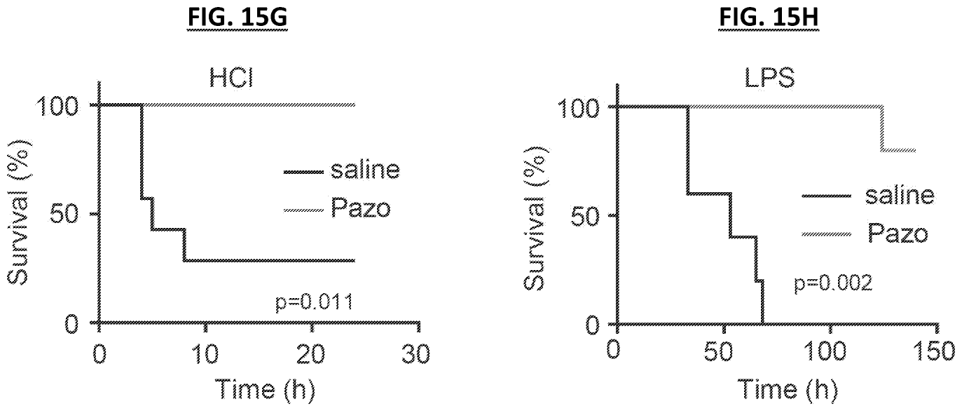

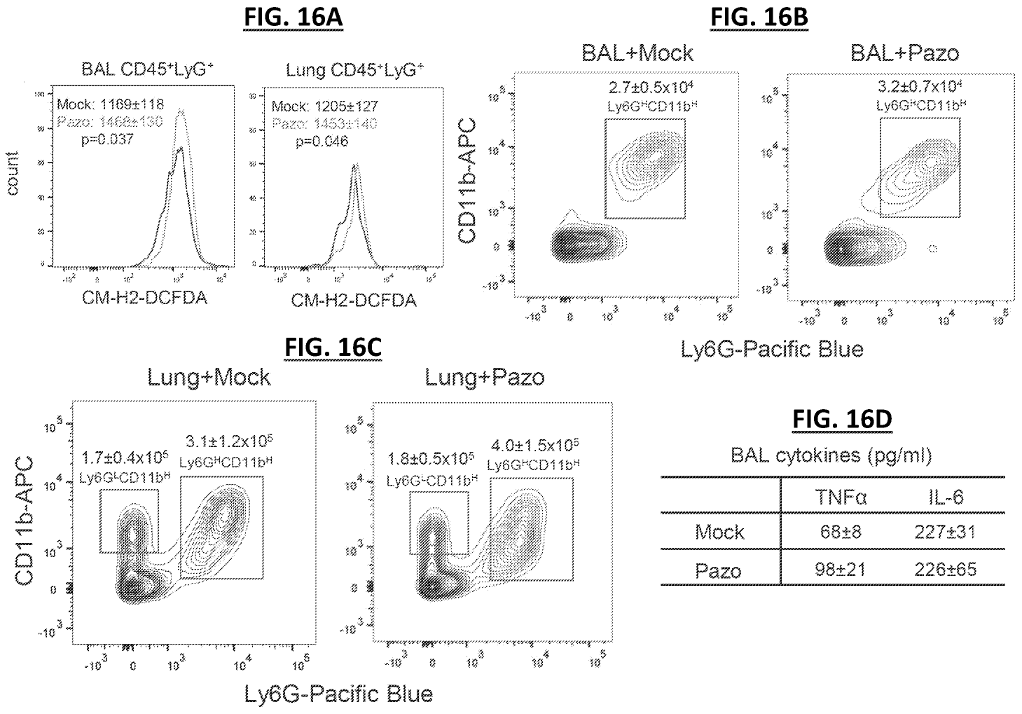

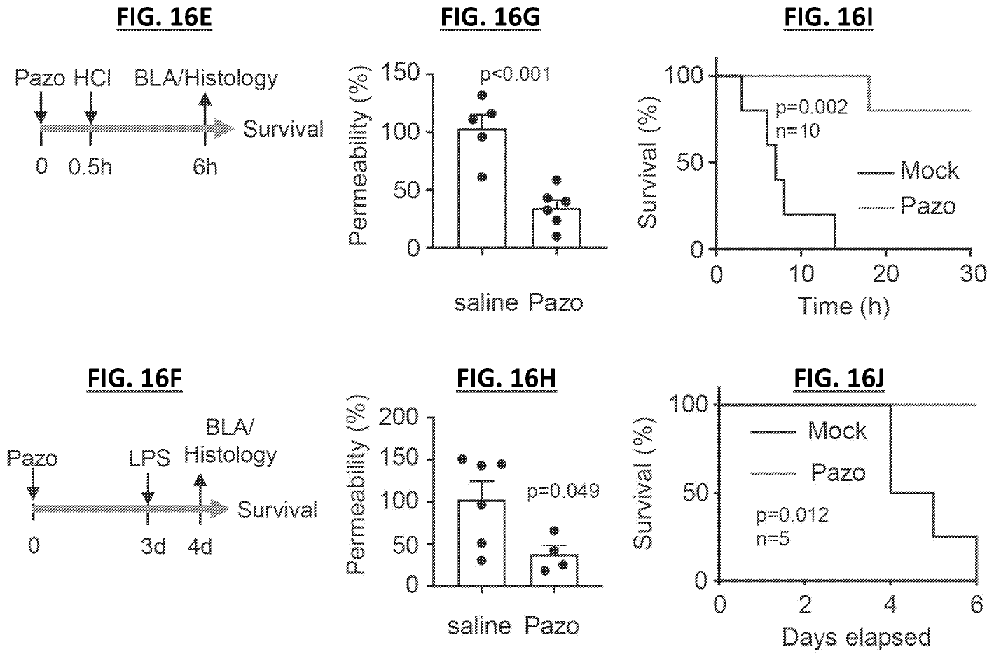

The effects of pazopanib were tested on both HCl- and LPS-induced ALI models. The test was first performed using the therapeutic modality where pazopanib was given intranasally post injury induction (FIGS. 15A and 15B). In this test, pazopanib treatment resulted in significant reductions in pulmonary permeability (FIGS. 15C and 15D), perivascular interstitial edema and lung injury index (FIGS. 15E and 15F), and mortality (FIGS. 15G and 15H). In addition, elevated ROS was detected in neutrophils in BALs and lungs of pazopanib-treated mice subjected to HCl lung insult (FIG. 16A). Consistently with the lack of effects on myeloid cell infiltration and cytokine contents observed with MAP3K2/3-deficiency, pazopanib did not affect these perimeters (FIGS. 16B-16D). In a prophylactic test, in which the drug was orally or intranasally administered prior to injury induction (FIGS. 16E and 16F), pazopanib also reduced lung permeability and mortality (FIGS. 16G-16J).

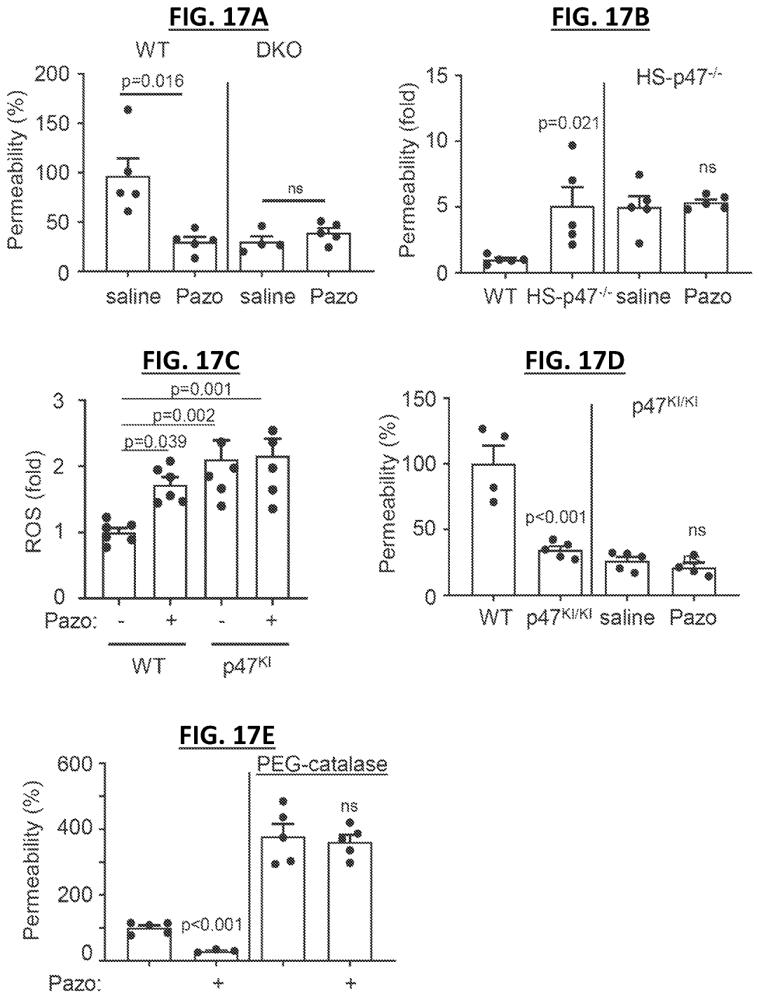

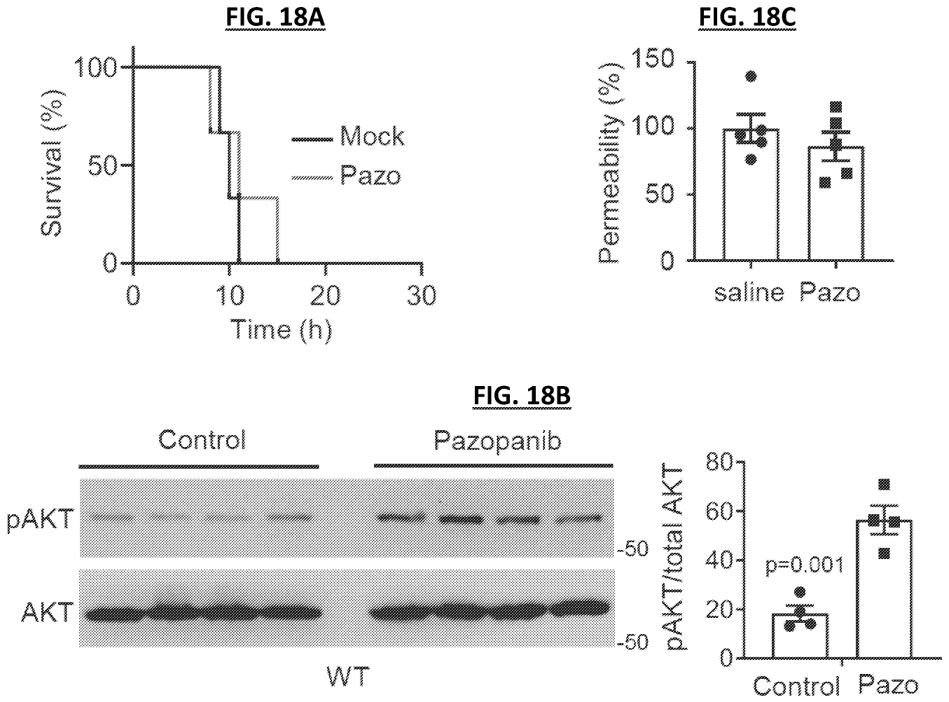

To determine whether pazopanib acts through the MAP3K2/3-p47^{phox} pathway, the effects of pazopanib were tested in the DKO mice; pazopanib lost its effect on pulmonary permeability in the DKO mice (FIG. 17A), suggesting that the action of pazopanib depends on MAP3K2 and 3. To test whether pazopanib regulates pulmonary permeability via p47^{phox}, the effects of pazopanib were examined with mice lacking p47^{phox} in hematopoietic cells. The mice were generated by transferring bone marrow from p47^{phox}-deficient mice to irradiated WT mice. Consistent with the hypothesis that neutrophil ROS provides beneficial effects on curbing ALI, hematopoietic deficiency of p47^{phox} increased pulmonary permeability in the HCl-induced ALI (FIG. 17B). Importantly, the lack of p47^{phox} in hematopoietic cells abrogated the effects of pazopanib on permeability (FIG. 17B) as well as on survival (FIG. 18A), suggesting that pazopanib acts through p47^{phox}. To further determine if pazopanib acts through p47^{phox} phosphorylation, pazopanib was tested with p47^{phox}-KI neutrophils and mice. Pazopanib failed to elevate ROS production in the p47^{phox}-KI neutrophils (FIG. 17C) or to reduce pulmonary permeability in the p47^{phox}-KI mice after HCl-induced lung injury (FIG. 17D), thus confirming that pazopanib acts through p47^{phox} phosphorylation at Ser-208. These results, together with the observation that pegylated catalase abrogated the effect of pazopanib in the ALI model (FIG. 17E), demonstrate that pazopanib acts though suppression of MAP3K2/3-mediated phosphorylation of p47^{phox} at S208 as well as through extracellular H₂O₂ to reduce pulmonary permeability.

Consistent with genetic inactivation of MAP3K2/3, pazopanib treatment led to increases in phosphorylated AKT levels in lung extracts subjected to ALI (FIG. 18B). Importantly, the treatment of mice with an AKT inhibitor (MK-2206) abrogated the effect of pazopanib on pulmonary permeability in the HCl-induced ALI model (FIG. 18C). These data together indicate that pazopanib acts through MAP3K2/3, p47^{phox}, and AKT to reduce pulmonary permeability during HCl-induced ALI.

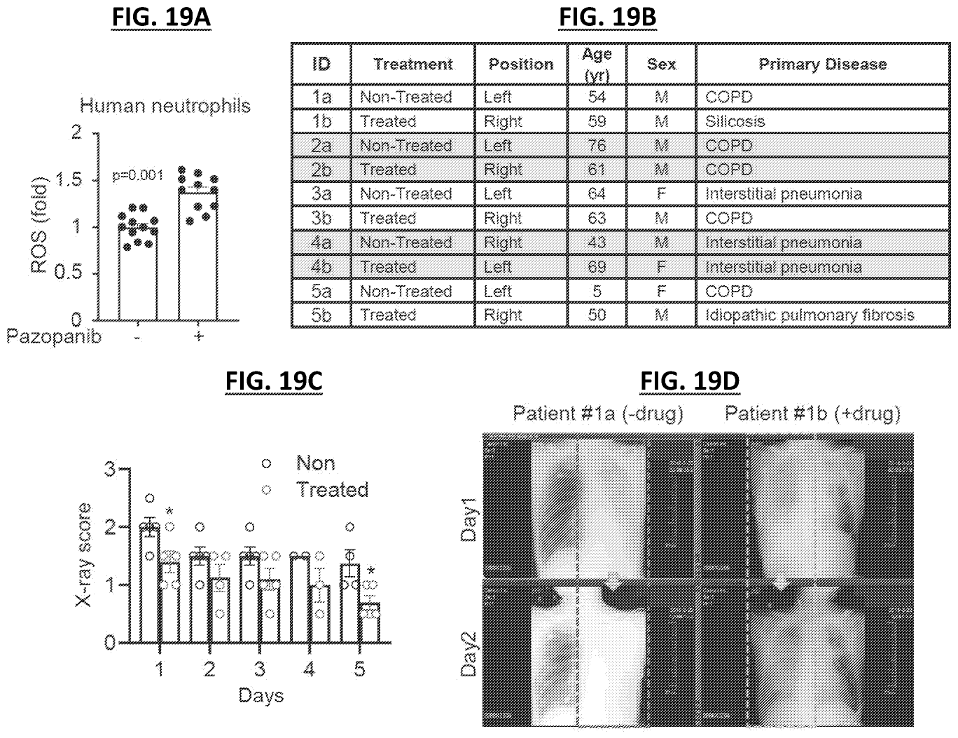

Both MAP3K2 and 3 proteins are expressed in human neutrophils. Together with the observation that pazopanib increases ROS production in human neutrophils (FIG. 19A), pazopanib has the real potential to treat ALI/ARDS in humans. Therefore, a preliminary human study was carried out to assess the effects of pazopanib in patients who underwent lung transplantation (LT). LT is an ideal human model for ALI, in which ALI/ARDS is caused by ischemia reperfusion with neutrophils being the key players and not confounded by infection and disease course. Five pairs of patients were enrolled in this preliminary clinical study, in which each pair received one lung from the same donor. One of the paired patients received an oral dose of pazopanib, whereas the other in the pair was untreated. The treated group and the control group were not significantly different in terms of age and sex (FIG. 19B). The treatment group had significantly lower X-ray opacity scores, which provide visual assessment of pulmonary edema, on the first day (difference: 0.6, 95% CI 0.04-1.26, p=0.04), and similar difference remained on the last day (0.7, 95% CI 0.1-1.3, p=0.04) (FIGS. 19C and 19D). On average, the treatment group had 0.5 point lower X-ray (p=0.02) compared to the control group. Thus, pazopanib treatment significantly reduced pulmonary edema in lung transplantation-induced injury.

### Selected Comments

In this study, evidence is provided demonstrating that pazopanib, an FDA-approved anti-cancer drug, abates ALI phenotypes in mice and human via a mechanism distinct from its anti-cancer action. It was shown that pazopanib is a potent inhibitor for MAP3K2/3-mediated phosphorylation of p47^{phox} at Ser-208 and strong mouse genetic evidence was presented to demonstrate that pazopanib acts largely through this MAP3K2/3-p47^{phox} pathway to ameliorate ALI despite it also inhibits tyrosine kinase receptors. This FDA-approved drug has been in clinic for years for cancer treatment and is well tolerated even for long term use. This safety profile, together with the unexpected substrate specificity of pazopanib towards p47^{phox} over the other MAP3K substrate MEK5, confers the drug an additional safety edge for treating ALI/ARDS. It thus has a promising potential to be the first therapeutic for ALI/ARD to fulfill the unmet medical need for pharmacological intervention of ALI/ARDS.

Numerous agents that were shown to work in mouse ALI models have failed in humans. These previously tested agents either act upstream in the process or target immune responses and inflammatory cytokines. Conversely, the mechanism of action of pazopanib described in this study, through a paracrine mechanism from neutrophils to pulmonary endothelial and epithelial cells, is likely conserved between human and mouse. The pilot human study showing that pazopanib reduces pulmonary edema in lung transplantation-induced injury is consistent with the conservation of the mechanism of action between species. Human studies can include a reformulation of pazopanib into an intravenous form and/or an enlargement of the study size. In addition, since the present study focuses on the acute phase of lung injuries in an aseptic setting, studies can be performed to investigate whether MAP3K2 and 3 inhibition is effective in ARDS caused by bacterial or viral infection and/or when being applied at the recovery phase of ARDS.

Excessive amounts of ROS generally cause damage to lipids, proteins, and DNA, particularly in cells that produce ROS. However, in this study, compelling evidence is provided to show that moderate increases in release of ROS from myeloid cells, via a paracrine mechanism, impact both pulmonary vascular and epithelial cells to favor enhancement of barrier functions. The co-culture experiments demonstrated that the changes in ROS release as results of the loss of MAP3K2/3 or p47^{phox} phosphorylation in neutrophils were sufficient for increasing AKT phosphorylation and enhancing barrier function in endothelial cells. Importantly, these effects were directly mediated by extracellular H₂O₂ rather than free superoxide radicals because of the effect of catalase. AKT activation leads to RAC1 activation in endothelial cells to regulate F-actin remodeling and enhance vascular integrity, providing an explanation to the effect of paracrine H₂O₂ on enhancement of endothelial cell integrity and reduction in permeability. The importance of AKT activation was further corroborated by the observation that its inhibitor abrogated beneficial effects of MAP3K2/3 inhibition on ALI (FIG. 18C).

The impact of paracrine H₂O₂ appears to go beyond of AKT regulation in lung endothelial cells. The scRNAseq data revealed broad transcriptional changes in pulmonary endothelial and epithelial cells that skew towards enhancement of barrier functions and epithelial survival and proliferation. These results, together with the immunohistostaining data, indicate that the moderate elevation of extracellular H₂O₂ released from neutrophils triggers the pulmonary microenvironment remodeling through crosstalk and interactions of different lung cell types, leading to protection of lungs from acute injuries. In this study, potential crosstalks contributed by endothelial and epithelial cells were studied. While not wishing to be limited by theory, it is believed that the oxidation of proteins such as PTEN, which would explain the activation of AKT by H₂O₂, could be one of the mechanisms of how H₂O₂ acts as a signaling molecule to exert broad effects on pulmonary barrier cells. In certain non-limting embodiments, the ALI protective effects of increased ROS production by inhibition of the MAP3K-p47 phosphorylation axis in LPS-induced model can be due to the combination of both AKT activation in barrier cells and anti-inflammatory effects of elevated H₂O₂.

### Example 3: Pazopanib Clinical Trial

### Composition of Pazopanib Formulation

A non-limiting formulation comprises pazopanib hydrochloride solubilized in hydroxypropyl betadex (HPB) and water for injection, prepared as an intravenous (IV) formulation. Each mL contains 5 mg of pazopanib hydrochloride and is solubilized with 200 mg of Hydroxypropyl Betadex USP and Water for Injection USP. The contents of the vial are diluted into 24 mL 0.9% Sodium Chloride Injection, USP (Normal Saline), or 5% glucose solution prior to infusion. The contents of one vial will supply 30 mg of pazopanib hydrochloride. The qualitative and quantitative composition is shown in Table 2 below.

TABLE 2
Components and Composition for pazopanib
hydrochloride injection

| | Amount per unit | | Quality |
|---|---|---|---|
| Component | (mg/mL) | Function | reference |
| Pazopanib | 5 mg/mL | Active | Formosa Labs |
| hydrochloride | | ingredient | |
| Hydroxypropyl | 200 mg/mL | Solubility | USP |
| betadex | | agent | |
| Water for injection | q.s. to 10 mL | Solvent | USP |
| Nitrogen* | N/A | Processing aid | NF |

*Used during compounding and vial filling as a processing aid to minimize headspace

Illustrative information for the drug substance impurities (name, structure, origin, and limit) is provided in Table 3.

TABLE 3
Name, structure, origin, and limits for Pazopanib HCl impurities

| Name | Structural formula | Control | Origin |
|---|---|---|---|
| DMIA HCl | 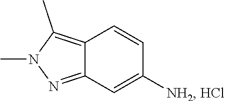 | NMT 1.7 ppm | Process impurity |
| AMBF-P | 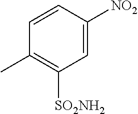 | NMT 1.7 ppm | Process impurity |
| Pazo-2 | 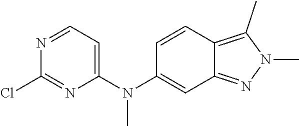 [SMILES: Cc1c2ccc(N(C)c3ccnc(Cl)n3)cc2nn1C] | NMT 1.7 ppm | Process impurity |
| DMIA HCl-P | 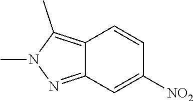 | NMT 1.7 ppm | Process impurity |
| Pazo-1 | 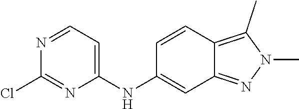 [SMILES: Cc1c2ccc(Nc3ccnc(Cl)n3)cc2nn1C] | NMT 0.10% | Degradation impurity |
| DCP | 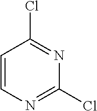 | NMT 0.10% | Degradation impurity |
| AMBF | 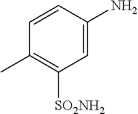 | NMT 0.10% | Process impurity |
| Pazopanib- C2 | 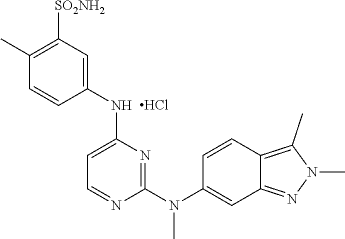 | NMT 0.10% | Degradation impurity |

Table 4 provides the chemical names for the impurities listed in Table 3.

TABLE 4
Chemical names for related substances

| Related substance | Chemical name |
|---|---|
| DCP | 2,4-dichloropyrimidine |
| AMBF | 5-amino-2-methylbenzene-sulfonamide |
| Pazo-1 | N-(2-chloropyrimidin-4-yl)-2,3-dimethyl-2H-indazol-6-amine |
| Pazopanib-C2 | 5-((2-((2,3-dimethy-2H-indazol-6-yl)methylamino]pyrimidin-4- |
| | yl]amino]-2-methylbenzenesulfonamide; hydrochloride |
| DMIA•HCl | 2,3-dimethyl-2H-indazol-6-amine hydrochloride |
| Pazo-2 | N-(2-chloropyrimidin-4-yl)-N,2,3-trimethyl-2H-indazol-6-amine |
| DMIA•HCl-P | 2,3-dimethyl-6-nitro-2H-indazole |
| AMBF-P | 2-methyl-5-nitrobenzene-sulfonamide |

### Pazopanib IV in the HCl-Induced Mouse Acute Lung Injury Model

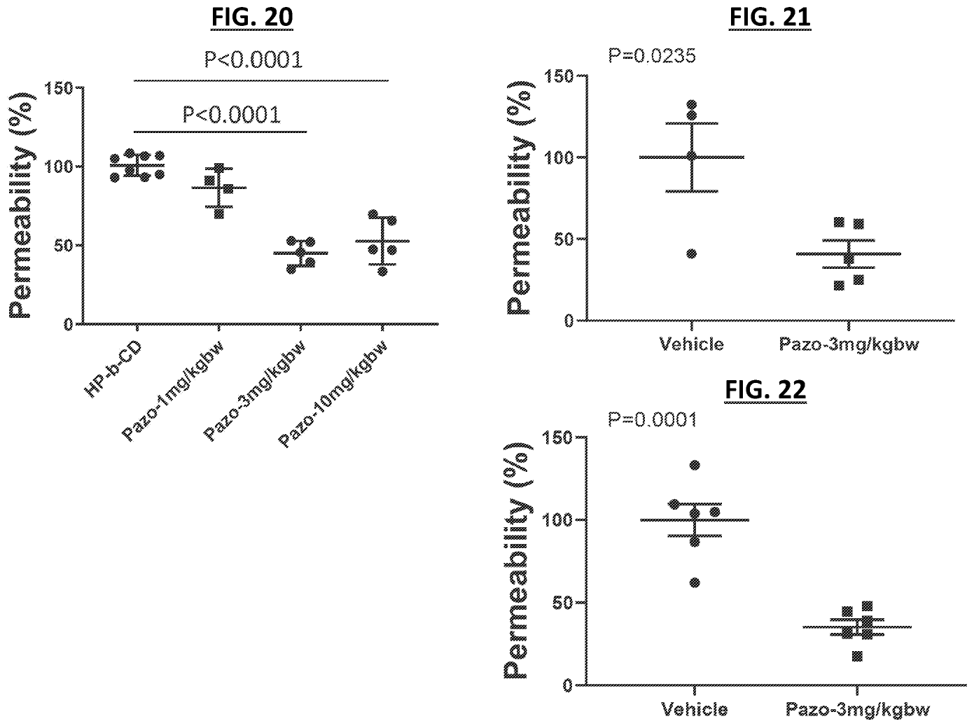

Based on in vitro results for inhibition of MAP3K2 and MAP3K3 phosphorylation of p47^{phox} at Ser-208 by pazopanib, the efficacy of pazopanib IV was determined at the doses of 1, 3, and 10 mg/kg body weight. BALB/c mice, 8 to 10 weeks old, were anaesthetized by Ketamine/Xylazine (100 and 10 mg/kg) and were kept under anesthesia during the whole procedure using Ketamine/Xylazine. After being deeply anesthetized (assessed by applying a noxious stimulus, e.g., toe pinch, and observing no reflex response and no change in either the rate or character of respiration), the mice were secured vertically from their incisors on a custom-made mount for orotracheal instillation. A 22G catheter was guided 1.5 cm below the vocal cords, and 2.5 μL/g of 0.05 M HCl was instilled. After the administration, the mice were monitored until their breathing gradually returned to normal. Then the mice were returned to the recovery cage on the heating pad and monitored for their anesthesia status. Half an hour before the induction of injury, Pazopanib IV, 1, 3, or 10 mg/kg body weight, or vehicle control was delivered to mice via the tail vein. Four hours after lung injury induction, 100 μL of FITC-labeled albumin (10 mg/mL) was injected via the retro-orbital vein. Two hours after FITC-albumin injection, mice were euthanized, and bronchoalveolar lavage (BAL) was collected via instilling 1 ml of PBS into the lungs, which was retrieved via a tracheal catheter. The green fluorescence of BAL was measured by a plate reader. BAL fluorescence intensities from pazopanib-treated mice were normalized to the intensities from vehicle-control-treated mice. A statistically significant reduction in permeability was observed at 3 mg/kg body weight pazopanib IV (P<0.0001) (FIG. 20).

### Pazopanib IV in the MHV-1 Mouse Model

After the efficacy of pazopanib IV in ameliorating lung damage was established in the HCl-induced ALI mouse model, its potential for reducing lung injury in a coronavirus infection-induced ALI mouse model was investigated. The murine hepatitis virus strain 1 (MHV-1) model was adopted for the pharmacology study. All MHV-1-infected A/J mice developed progressive interstitial edema, neutrophil/macrophage infiltrates, and hyaline membranes, leading to the death of all mice. Two studies have been completed to date. In the first one, mice were administered a 3-dose regimen of the study intervention at 6, 21, and 32 hours after virus inoculation. In the second, mice were administered a 2-dose regimen at 24 and 33 hours after inoculation.

### Study 1:

A/J mice, 8 to 10 weeks old, were anaesthetized by Ketamine/Xylazine (100 and 10 mg/kg) and were kept under anesthesia during the whole procedure using Ketamine/Xylazine. After being deeply anesthetized (assessed by applying a noxious stimulus, e.g., toe pinch, and observing no reflex response and no change in either the rate or character of respiration), mice received an intranasal inoculation of 5000 PFU MHV-1 in 20 μL Dulbecco's modified Eagle's medium. After the administration, the mice were monitored until their breathing gradually returned to normal. Then the mice were returned to the recovery cage on the heating pad and monitored for their anesthesia status. Three doses of pazopanib IV, 3 mg/kg body weight, or vehicle control were then delivered to mice via the retro-orbital vein including one each at 6, 21, and 32 hours after virus inoculation. Fifteen hours after the 3rd dose of the intervention, 100 μl of FITC-labeled albumin (10 mg/mL) was injected via the retro-orbital vein. Two hours after FITC-albumin injection, mice were euthanized and bronchoalveolar lavage (BAL) was collected via instilling 1 mL of PBS into the lungs, which was retrieved via a tracheal catheter. The green fluorescence of BAL was measured by the plate reader. BAL fluorescence intensities from pazopanib-treated mice were normalized to the intensities from vehicle-control-treated mice wherein higher intensity corresponds to greater permeability. A significant reduction in permeability was observed at 3 mg/kg body weight pazopanib IV (P=0.0235) (FIG. 21).

### Study 2:

A/J mice, 8-10 weeks old, were anaesthetized by Ketamine/Xylazine (100 and 10 mg/kg) and were kept under anesthesia during the whole procedure using Ketamine/Xylazine. After being deeply anesthetized (assessed by applying a noxious stimulus, e.g., toe pinch, and observing no reflex response and no change in either the rate or character of respiration), mice received an intranasal inoculation of 6000 PFU MHV-1 in 20 μL Dulbecco's modified Eagle's medium. After the administration, the mice were monitored until their breathing gradually returned to normal. Then the mice were returned to the recovery cage on the heating pad and monitored for their anesthesia status. Two doses of pazopanib IV, 3 mg/kg body weight, or vehicle control were then delivered to mice via the retro-orbital vein, including one each at 24 and 33 hours after virus inoculation. Sixteen hours after the second dose of the study intervention (pazopanib IV or placebo), 100 μL of FITC-labeled albumin (10 mg/mL) was injected via the retro-orbital vein. Two hours after FITC-albumin injection, mice were euthanized and bronchoalveolar lavage (BAL) was collected via instilling 1 mL of PBS into the lungs, which was retrieved via a tracheal catheter. The green fluorescence of BAL was measured by the plate reader. BAL fluorescence intensities from pazopanib-treated mice were normalized to the intensities from vehicle-control-treated mice wherein higher intensity corresponds to greater permeability. A significant reduction in permeability was observed at 3 mg/kg body weight Pazopanib IV (P=0.0001) (FIG. 22).

### Estimation of the Maximum Safe Starting Dose in Clinical Trials

In the mouse coronavirus-induced lung injury model, the efficacious dose was approximately 3 mg/kg (Section 8.2.1), which translates to a human equivalent dose of 3×0.08 mg or 0.24 mg/kg. Assuming an average human weighs 70 kg, the projected clinical efficacious dose would be approximately 16.8 mg. A starting dose of 20 mg is proposed for study in COVID-19 patients based on the justification presented below. VOTRIENT® (pazopanib) has been approved in tablet form for oral administration since 2009 for the treatment of advanced renal cell carcinoma and soft tissue sarcoma. The recommended dosage of pazopanib at 800 mg orally once daily in cancer patients was well-tolerated. The oral bioavailability of pazopanib was reported to be 21.4% (13.5% to 38.9%) with a Cₘₐₓ of 43.9 μg/mL and AUC of 806 μg·h/mL.

### NOAEL and Human Equivalent Dose Calculation

It was concluded from the 2-week GLP IV infusion (20-minute) studies that the NOAEL (no observed adverse effect level) was 10 mg/kg/day in both rats and monkeys (Table 5). Accordingly, the corresponding human equivalent doses (HED) for rat and monkey would be 1.6 and 3.2 mg/kg, respectively. Assuming the body weight of a human is 70 kg, this corresponds to 112 and 224 mg HED for rat and monkey, respectively. Therefore, the margins of safety based on the 2-week rat and monkey studies would be 5.6-fold and 11.2-fold, respectively (Table 5).

TABLE 5
MSSD safety margin approach (HED
of NOAEL from toxicology studies)

| | | | | HED of | Margin |
|---|---|---|---|---|---|
| | | Route of | NOAEL | NOAEL | of |
| Species | Duration | administration | (mg/kg) | (mg/kg) | safety* |
| Rat | Single | IV bolus | 30 | 4.8 | 16.6X |
| | dose | | | | |
| Rat | 5 days | IV (20 minute | 50 | 8 | 27.6X |
| | | infusion) | | | |
| Rat | 14 days | IV (20 minute | 10 | 1.6 | 5.6X |
| | | infusion) | | | |
| Monkey | 5 days | IV (20 minute | 30 | 9.6 | 33.1X |
| | | infusion) | | | |
| Monkey | 14 days | IV (20 minute | 10 | 3.2 | 11.2X |
| | | infusion) | | | |

*Assuming proposed clinical dose is 20 mg/day or 0.29 mg/kg.

### Clinical Pharmacokinetics Versus Animal Toxicokinetics

This justification is based upon the results of the GLP 2-week IV infusion toxicology and toxicokinetic studies in rats and monkeys (Table 6). Based on clinical PK and toxicology results, it was suggested that 20 mg/subject would be a safe starting dose for the Phase 2 clinical trial. After an IV administration of pazopanib at 5 mg/subject (N=7), the AUC and Cₘₐₓ were 20.4 μg·h/mL and 0.848 μg/mL, respectively. Assuming dose-linearity, AUC and Cₘₐₓ would be 81.6 μg·h/mL and 3.4 μg/mL, respectively, after an IV injection of 20 mg/subject. These extrapolated values were likely to be exaggerated and the calculated margins of safety would be higher accordingly.

Plasma drug concentration analyses from the 2-week GLP toxicokinetic studies showed the Cₘₐₓ values were 55 and 47 μg/mL in male and female rats, respectively. The safety margins derived by dividing the Cₘₐₓ (3.4 μg/mL) in man after an IV administration of pazopanib (20 mg) was calculated to be 13.8-fold and 16.2-fold in male and female rats, respectively (Table 6).

Similarly, the safety margins of pazopanib based on the Cₘₐₓ values from the male and female monkeys were calculated to be 25-fold and 23-fold, respectively. However, the margin of safety based on AUC values were <10-fold. This may be due to the relatively long half-life (t_{1/2}) of pazopanib in man (27.5 hours) versus t_{1/2} of a few hours in rats and monkeys. In addition, the NOAELs used in the calculation of safety margins based on the rat and monkey study were determined after 14 days of IV dosing as compared with the starting clinical dose of 20 mg, which will be administered to COVID-19 patients as a single infusion in Part 1 of the study. Lastly, the plasma drug concentrations after 800 mg oral daily dose of pazopanib in cancer patients (maximum recommended human dose, MRHD) were significantly higher than those observed in the GLP rat and monkey toxicology studies. The oral bioavailability of pazopanib is approximately 20%, which implies that an IV dose of up to 160 mg may be tolerated in the clinical trials. Based on the rationale elaborated above, it is concluded that the proposed starting dose of 20 mg of pazopanib IV for the Phase 2 clinical trial in COVID-19 patients should be safe and well-tolerated, and the maximum clinical exposure levels planned may not exceed that allowed for the MRHD per the label for the FDA-approved VOTRIENT product.

TABLE 6
Margin of safety based on clinical PK and
2-Week GLP rat and monkey TK studies

| | | | | Cₘₐₓ at | | AUC_{(0-∞)} at | |
|---|---|---|---|---|---|---|---|
| | | | | NOAEL | Margin of | NOAEL | Margin of |
| Species | Gender | | Route | μg/mL | safety | μg · hr/mL | safety |
| Rat | M | | IV | 55 | 16.2 | 277.8 | 3.4 |
| Rat | F | | IV | 47 | 13.8 | 276.5 | 3.4 |
| Monkey | M | | IV | 85 | 25.0 | 143 | 1.75 |
| Monkey | F | | IV | 80 | 23.5 | 167 | 2.05 |
| Human^{a} | 20 | mg^{b} | IV | 3.4^{a} | | 81.6^{a} | |
| | 800 | mg^{c} | PO | 58.1 | | 1037 | |

^{a}Cₘₐₓ after IV administration of 5 mg/day of pazopanib was 0.85 μg/mL and AUC_{(0-∞)} was 20.4 μg*h/mL.
^{b}Assuming dose-linearity, Cₘₐₓ and AUC_{(0-∞)} at 20 mg/kg would be 3.4 μg/mL and 81.6 μg*h/mL, respectively.
^{c}Information obtained from the label for the FDA-approved VOTRIENT product.

### Overall Study Design

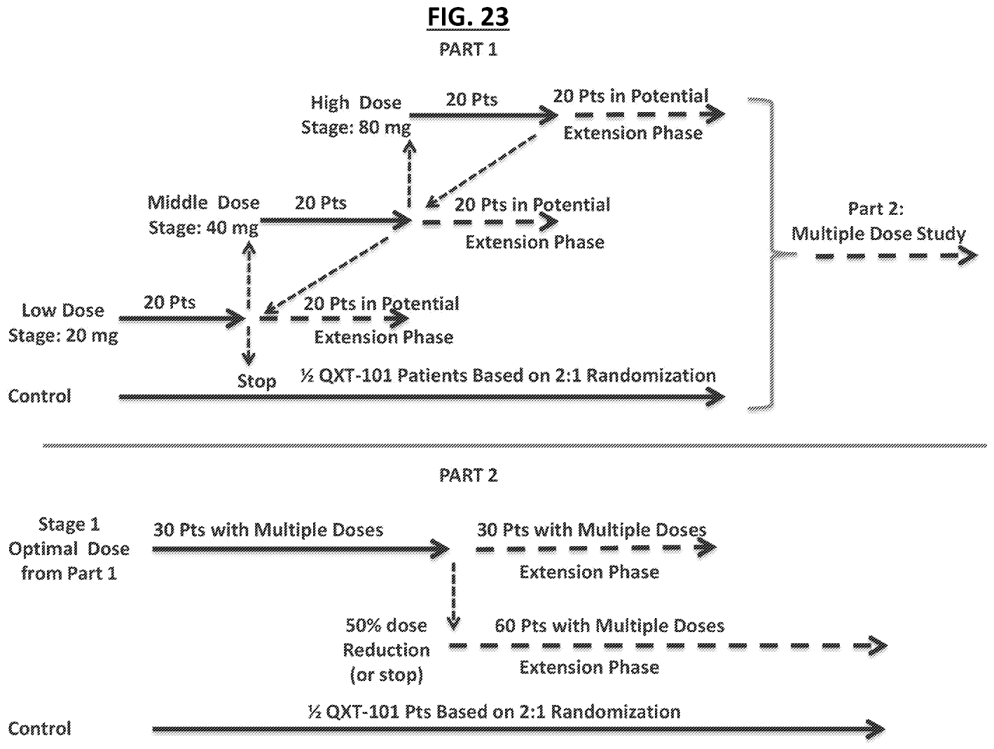

No clinical investigations have been completed or are ongoing as part of any development program for pazopanib IV. The opening study is a Phase 2, double-blind, multicenter, 2-arm, randomized, placebo-controlled, 2-part, adaptive trial investigating the safety, tolerability, and PK of single and multiple dosing with pazopanib IV in hospitalized participants with confirmed COVID-19. Part 1 follows a single ascending dose (SAD) design intended to identify the potential optimal dose to be utilized directly in the second part (Part 2) as a multiple-dose (MD) regimen. The study also looks for a preliminary efficacy signal that would indicate improvement in gas exchange in this population. A graphic of the overall design is presented in FIG. 23 along with a comprehensive description and statistical methods. Pharmacokinetic assessments are conducted in both parts of the study. Results from these investigations help to characterize the single dose and multiple dose safety and PK profile of pazopanib IV in COVID-19 patients to inform future studies.

The screening period lasts 1 to 3 days (inclusive of study Day 1). Prospective candidates will be evaluated according to the inclusion and exclusion criteria to determine eligibility.

Eligible candidates are then enrolled in the study and randomly assigned to either the experimental (pazopanib IV) or control (placebo) arm in a 2:1 ratio, respectively. To avoid population bias, randomization is controlled by stratification based on disease severity (ICU vs non-ICU hospitalization). For both arms, treatment includes study interventions along with standard of care. In Part 1 of the study (SAD), interventions are administered as a single 20-minute infusion (peripheral or central cannula) after randomization. Two infusions are administered in Part 2 (Day 1 and 3) with an option for a third dose (Day 5). Participants undergo a series of in-patient study assessments. Daily evaluations are performed while participants remain in the hospital, unless otherwise noted. After discharge, weekly follow-up by telecommunication is arranged on the designated days, as appropriate. The overall duration for each participant is 28 to 34 days, which includes a 1- to 3-day screening period and a 30-day (±2 days) observation period.

### Study Population

As described above, recent studies in mouse ALI models suggest that pazopanib moderates the development of ALI and ARDS by inhibiting protein kinases MAP3K2 and MAP3K3 in neutrophils, which are key factors in the development of ARDS and likely COVID-19 related ARDS. Because ALI/ARDS is central to the pathophysiology of COVID-19, the study population is expected to be clinically relevant and meaningful for assessment of the investigational study drug.

The primary eligibility criterion for the Phase 2 study is hospitalization with confirmed SARS-CoV-2 infection and clinical signs suggestive of progressive COVID-19. Two additional disease-related criteria include radiographic and blood gas assessments. The presence of radiographic bilateral infiltrates, visualized as opacities by chest imaging, is a common feature of patients with ARDS and COVID-19. A second qualifying criterion is the level of oxygen support needed to maintain 92% blood 02 saturation by pulse oximetry (SpO₂). The requirement is at least 5 L (40% FiO₂) or greater. These values correspond to a maximum imputed PaO₂/FiO₂ ratio of 160. Alternatively, eligible participants may be on invasive mechanical ventilation at screening. These criteria restrict the study population to those with moderate to severe lung injury (Berlin definition, moderate ARDS: 100 to 200 [PEEP≥5 cm H₂O]). Based on the proposed MoA, the optimal window of intervention is the time during which the viral infection triggers the hyper-inflammatory response that is associated with the onset of significant lung injury and gas exchange impairment in the more critically ill patients. Therefore, without wishing to be limited by any theory, this therapy can have a favorable risk/benefit profile for patients whose lung function has deteriorated to the point where they may soon become candidates for, or have recently progressed to, invasive mechanical ventilation.

### Selected Comments

Pazopanib is an angiogenesis inhibitor that the FDA approved as a treatment of advanced renal cell carcinoma and which has been demonstrated herein to be an effective treatment for the pathophysiology related to COVID-19. The recent COVID-19 pandemic has caused a sudden and substantial global increase in hospitalization for pneumonia with multiorgan diseases. Pazopanib IV has demonstrated efficacy in the coronavirus infection-induced mouse model as well as an acid-induced ALI mouse model via IV injection at 3 mg/kg, which is equivalent to 0.24 mg/kg HED. Assuming each patient weighs an average of 70 kg, the clinical efficacious dose is projected to be 16.8 mg/day (or approximately 20 mg/day).

VOTRIENT® (pazopanib) at 800 mg orally once daily has been shown to be well tolerated in cancer patients. The oral bioavailability of pazopanib was approximately 20%, suggesting that an IV dose of pazopanib up to 160 mg could be well tolerated in patients. This was supported by the 2-week IV infusion studies in rats and monkeys. The plasma drug concentrations and toxicokinetic parameters derived from these GLP rat and monkey studies after IV administration at the STD10/NOAEL dose of 10 mg/kg/day were similar to the published systemic exposures of pazopanib at the NOAEL doses after oral administration to rats and monkeys and below those observed in cancer patients after oral administration of VOTRIENT® at 800 mg/day. The 2-week monkey study demonstrated a safety margin of 11.2-fold compared to the planned clinical starting dose. This safety margin increased to 33.1-fold in a prior IV dose range finding study when pazopanib IV was administered to monkeys daily for 5 consecutive days.

### Enumerated Embodiments

The following exemplary embodiments are provided, the numbering of which is not to be construed as designating levels of importance:

Embodiment 1 provides a method of treating, ameliorating, and/or preventing post-stroke brain ischemia-reperfusion injury (IRI) in a subject in need thereof, the method comprising administering to the subject a therapeutically effective amount of pazopanib, or a salt or solvate thereof.

Embodiment 2 provides a method of treating, ameliorating, and/or preventing ischemia-reperfusion injury (IRI) not caused by post-stroke brain ischemia, lung injury related to a coronavirus infection, acute lung injury (ALI), and/or acute respiratory distress syndrome (ARDS) in a subject in need thereof, the method comprising administering to the subject a therapeutically effective amount of pazopanib, or a salt or solvate thereof.

Embodiment 3 provides the method of any one of Embodiments 1-2, wherein the subject is in an intensive care unit (ICU) or emergency room (ER).

Embodiment 4 provides the method of any one of Embodiments 1-3, wherein the subject is further administered at least one additional agent and/or therapy that treats, ameliorates, prevents, and/or reduces one or more symptoms of the IRI, lung injury related to the coronavirus infection, ALI, and/or ARDS.

Embodiment 5 provides the method of any one of Embodiments 1-4, wherein the administration route is selected from the group consisting of oral, intracranial, nasal, rectal, parenteral, sublingual, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

Embodiment 6 provides the method of any one of Embodiments 1-5, wherein the pazopanib, or a salt or solvate thereof, is administered to the subject at a frequency selected from the group consisting of about three times a day, about twice a day, about once a day, about every other day, about every third day, about every fourth day, about every fifth day, about every sixth day and about once a week.

Embodiment 7 provides the method of any one of Embodiments 1-6, wherein the pazopanib, or a salt or solvate thereof, is administered to the subject after reperfusion takes place.

Embodiment 8 provides the method of any one of Embodiments 1-7, wherein administration of the pazopanib, or a salt or solvate thereof, to the subject does not cause at least one significant adverse reaction, side effect and/or toxicity associated with administration of the pazopanib, or a salt or solvate thereof, to a subject suffering from cancer.

Embodiment 9 provides the method of Embodiment 8, wherein the at least one adverse reaction, side effect and/or toxicity is selected from the group consisting of hepatotoxicity, prolonged QT intervals and torsades de pointes, hemorrhagic event, decrease or hampering of coagulation, arterial thrombotic event, gastrointestinal perforation or fistula, hypertension, hypothyroidism, proteinuria, diarrhea, hair color changes, nausea, anorexia, and vomiting.

Embodiment 10 provides the method of any one of Embodiments 1-9, wherein the subject is dosed with an amount of pazopanib, or a salt or solvate thereof, that is lower than the amount of pazopanib, or a salt or solvate thereof, with which a subject suffering from cancer is dosed for cancer treatment.

Embodiment 11 provides the method of any one of Embodiments 1-10, wherein the subject is a mammal.

Embodiment 12 provides the method of any one of Embodiments 1-11, wherein the mammal is a human.

Embodiment 13 provides the method of any of Embodiments 1-12, wherein the subject is intravenously dosed with between about 5 mg and about 100 mg of an amount of pazopanib, or a salt or solvate thereof.

Embodiment 14 provides a kit comprising pazopanib, or a salt or solvate thereof, an applicator, and an instructional material for use thereof, wherein the instructional material comprises instructions for treating, ameliorating, and/or preventing ischemia-reperfusion injury (IRI), a lung injury related to a coronavirus infection, acute lung injury (ALI), and/or acute respiratory distress syndrome (ARDS) in a subject.

Embodiment 15 provides the kit of Embodiment 14, further comprising at least one additional agent that treats, prevents, or reduces one or more symptoms of the IRI, the lung injury related to the coronavirus infection, ALI, and/or ARDS.

Embodiment 16 provides a method of evaluating efficacy of a drug in treating ischemia-reperfusion injury (IRI), lung injury related to a coronavirus infection, acute lung injury (ALI), or acute respiratory distress syndrome (ARDS), the method comprising contacting a neutrophil with the drug and measuring neutrophil ROS production levels after the contacting, wherein, if the neutrophil ROS production levels increase after the contacting, the drug is efficacious in treating IRI, lung injury related to the coronavirus infection, ALI, and/or ARDS.

Embodiment 17 provides a method of evaluating efficacy of a drug in treating a subject suffering from ischemia-reperfusion injury (IRI), lung injury related to a coronavirus infection, acute lung injury (ALI), and/or acute respiratory distress syndrome (ARDS), the method comprising (i) measuring neutrophil ROS production levels in the subject after being administered the drug, wherein, if the neutrophil ROS production levels in the subject after being administered the drug are higher than the neutrophil ROS production levels in the subject before being administered the drug, the drug is efficacious in treating IRI, lung injury related to the coronavirus infection, ALI, or ARDS in the subject; or (ii) measuring H₂O₂ levels in the lungs of the subject after being administered the drug, wherein, if the H₂O₂ levels in the lungs of the subject after being administered the drug are higher than the H₂O₂ levels in the lungs of the subject before being administered the drug, the drug is efficacious in treating lung injury related to the coronavirus infection, ALI or ARDS in the subject.

Embodiment 18 provides the method of any one of Embodiments 1-13, 16, and 17, wherein the coronavirus infection is COVID-19.

Embodiment 19 provides the kit of Embodiment 14 or 15, wherein the coronavirus infection is COVID-19.

The disclosures of each and every patent, patent application, and publication cited herein are hereby incorporated herein by reference in their entirety.

While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of treating, ameliorating, or preventing at least one condition selected from the group consisting of post-stroke brain ischemia-reperfusion injury (IRI), ischemia-reperfusion injury (IRI) not caused by post-stroke brain ischemia, lung injury related to a coronavirus infection, acute lung injury (ALI), and acute respiratory distress syndrome (ARDS) in a subject in need thereof,
the method comprising administering to the subject a therapeutically effective amount of pazopanib, or a salt or solvate thereof.

2. (canceled)

3. The method of claim 1, wherein the subject is in an intensive care unit (ICU) or emergency room (ER).

4. The method of claim 1, wherein the subject is further administered at least one additional agent and/or therapy that treats, ameliorates, prevents, or reduces one or more symptoms of the IRI, lung injury related to the coronavirus infection, ALI, or ARDS.

5. The method of claim 1, wherein the administration route is selected from the group consisting of oral, intracranial, nasal, rectal, parenteral, sublingual, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

6. The method of claim 1, wherein the pazopanib, or a salt or solvate thereof, is administered to the subject at a frequency selected from the group consisting of about three times a day, about twice a day, about once a day, about every other day, about every third day, about every fourth day, about every fifth day, about every sixth day and about once a week.

7. The method of claim 1, wherein the pazopanib, or a salt or solvate thereof, is administered to the subject after reperfusion takes place.

8. The method of claim 1, wherein administration of the pazopanib, or a salt or solvate thereof, to the subject does not cause at least one significant adverse reaction, side effect or toxicity associated with administration of the pazopanib, or a salt or solvate thereof, to a subject suffering from cancer.

9. The method of claim 8, wherein the at least one adverse reaction, side effect or toxicity is selected from the group consisting of hepatotoxicity, prolonged QT intervals and torsades de pointes, hemorrhagic event, decrease or hampering of coagulation, arterial thrombotic event, gastrointestinal perforation or fistula, hypertension, hypothyroidism, proteinuria, diarrhea, hair color changes, nausea, anorexia, and vomiting.

10. The method of claim 1, wherein the subject is dosed with an amount of pazopanib, or a salt or solvate thereof, that is lower than the amount of pazopanib, or a salt or solvate thereof, with which a subject suffering from cancer is dosed for cancer treatment.

11. The method of claim 1, wherein the subject is a mammal.

12. The method of claim 1, wherein the mammal is a human.

13. The method of claim 1, wherein the subject is intravenously dosed with between about 5 mg and about 100 mg of an amount of pazopanib, or a salt or solvate thereof.

14. A kit comprising pazopanib, or a salt or solvate thereof, an applicator, and an instructional material for use thereof, wherein the instructional material comprises instructions for treating, ameliorating or preventing ischemia-reperfusion injury (IRI), a lung injury related to a coronavirus infection, acute lung injury (ALI), or acute respiratory distress syndrome (ARDS) in a subject.

15. The kit of claim 14, further comprising at least one additional agent that treats, prevents, or reduces one or more symptoms of the IRI, the lung injury related to the coronavirus infection, ALI, or ARDS.

16. A method of evaluating efficacy of a drug in treating ischemia-reperfusion injury (IRI), lung injury related to a coronavirus infection, acute lung injury (ALI), or acute respiratory distress syndrome (ARDS),
the method comprising contacting a neutrophil with the drug and measuring neutrophil ROS production levels after the contacting, wherein, if the neutrophil ROS production levels increase after the contacting, the drug is efficacious in treating IRI, lung injury related to the coronavirus infection, ALI, or ARDS.

17. A method of evaluating efficacy of a drug in treating a subject suffering from ischemia-reperfusion injury (IRI), lung injury related to a coronavirus infection, acute lung injury (ALI), or acute respiratory distress syndrome (ARDS), the method comprising
(i) measuring neutrophil ROS production levels in the subject after being administered the drug, wherein, if the neutrophil ROS production levels in the subject after being administered the drug are higher than the neutrophil ROS production levels in the subject before being administered the drug, the drug is efficacious in treating TRI, lung injury related to the coronavirus infection, ALI, or ARDS in the subject; or
(ii) measuring H₂O₂ levels in the lungs of the subject after being administered the drug, wherein, if the H₂O₂ levels in the lungs of the subject after being administered the drug are higher than the H₂O₂ levels in the lungs of the subject before being administered the drug, the drug is efficacious in treating lung injury related to the coronavirus infection, ALI or ARDS in the subject.

18. The method of claim 1, wherein the coronavirus infection is COVID-19.

19. The kit of claim 14, wherein the coronavirus infection is COVID-19.


SEQUENCE LISTING (5 sequences)

SEQ ID NO: 1 - protein, 14 aa, Artificial Sequence
  Chemically Synthesized
  misc_feature (8)..(8): Xaa = phosphoserine
KRGWVPAXYLEPLD

SEQ ID NO: 2 - DNA, 33 bp, Artificial Sequence
  Chemically Synthesized
ggttgggtccctgcatcctatctggagcccctt

SEQ ID NO: 3 - protein, 11 aa, Artificial Sequence
  Chemically Synthesized
GWVPASYLEPL

SEQ ID NO: 4 - DNA, 33 bp, Artificial Sequence
  Chemically Synthesized
ggttgggtccctgcagcttacctcgagcccctt

SEQ ID NO: 5 - protein, 11 aa, Artificial Sequence
  Chemically Synthesized
GWVPAAYLEPL